(19) United States
(12) Patent Application Publication
(10) Pub. No.: US 2009/0068182 A1
(43) Pub. Date: Mar. 12, 2009

(54) Cytotoxicity mediation of cells evidencing surface expression of CD9

(75) Inventors: David S. F. Young, Toronto (CA); Helen P. Findlay, Toronto (CA); Susan E. Hahn, Toronto (CA); Lisa M. Cechetto, Seoul (KR); Alison L. Ferry, Thornhill (CA)

Correspondence Address: MCHALE & SLAVIN, P.A., 2855 PGA BLVD, PALM BEACH GARDENS, FL, 33410, US

(73) Assignee: Arius Research Inc.

(21) Appl. No.: 12/228,682

(22) Filed: Aug. 13, 2008

Related U.S. Application Data
(60) Provisional application No. 61/124,019, filed on Apr. 11, 2008.
(60) Provisional application No. 61/026,584, filed on Feb. 6, 2008.
(60) Provisional application No. 60/965,165, filed on Aug. 17, 2007.

(51) Int. Cl. A61K 39/395; C07K 16/30; A61P 35/00; G01N 33/574; C12N 5/12

(52) U.S. Cl. 424/133.1; 530/388.1; 530/387.3; 435/346; 424/141.1; 424/178.1; 435/7.1; 530/388.22

(57) ABSTRACT

This invention relates to the staging, diagnosis and treatment of cancerous diseases (both primary tumors and tumor metastases), particularly to the mediation of cytotoxicity of tumor cells; and most particularly to the use of cancerous disease modifying antibodies (CDMAB), optionally in combination with one or more CDMAB/chemotherapeutic agents, as a means for initiating the cytotoxic response. The invention further relates to binding assays, which utilize the CDMAB of the instant invention. The anti-cancer antibodies can be conjugated to toxins, enzymes, radioactive compounds, cytokines, interferons, target or reporter moieties and hematogenous cells.

### CROSS-REFERENCE TO RELATED APPLICATIONS

This application claims the benefit of the filing date of U.S. Provisional Patent Application No. 61/124,019, filed Apr. 11, 2008, U.S. Provisional Patent Application No. 61/026,584, filed Feb. 6, 2008, and U.S. Provisional Patent Application No. 60/965,165, filed Aug. 17, 2007, the contents of which are herein incorporated by reference herein.

### FIELD OF THE INVENTION

This invention relates to the diagnosis and treatment of cancerous diseases, particularly to the mediation of cytotoxicity of tumor cells; and most particularly to the use of cancerous disease modifying antibodies (CDMAB), optionally in combination with one or more CDMAB/chemotherapeutic agents, as a means for initiating the cytotoxic response. The invention further relates to binding assays, which utilize the CDMAB of the instant invention.

### BACKGROUND OF THE INVENTION

The cell membrane contains many different cell-surface proteins, some in motion and some anchored to the cytoskeleton. This huge repertoire of cell-surface proteins is capable of executing different functions such as signaling and adhesion. It is also known that certain types of membrane proteins are responsible for the organization of these cell-surface proteins into complexes capable of united functions that they could not carry out as single molecules. This emerging family of proteins, the tetraspanins or transmembrane 4 (TM4) family of integral membrane proteins, serves as a molecular facilitator or organizer of multi-molecular complexes.

Tetraspanins have been implicated in a large variety of physiological processes such as immune cell activation, cell migration, cell-cell fusion (including fertilization) and various aspects of cellular differentiation. These molecules have also been shown to play a role in infectious diseases (e.g. malaria, hepatitis C and human immunodeficiency virus) and several genetic diseases are linked to mutations in these molecules (e.g. X-linked mental retardation, retinal degeneration and incorrect assembly of human basement membranes in the kidney and skin) (Boucheix and Rubinstein. Cell. Mol. Life. Sci. 58(9):1189-1205 2001). The ability of tetraspanins to interact with many other signaling molecules and participate in activation, adhesion and cell differentiation all relate to its role as “molecular facilitators” that bring together large molecular complexes and allow them, through stabilization, to function more efficiently. The interaction of tetraspanins with other signaling molecules is sometimes referred to as the tetraspanin web.

This super family (TM4SF) was first recognized in 1990, when comparison of the sequences of the newly cloned CD37, CD81 (TAPA-1) and sm23 genes with the tumor-associated gene CD63 (ME491) (Hotta et al. Cancer Res. 48(11):2955-2962 1988) revealed sequence homology and a conserved predicted structure (Wright et al. J Immunol 144(8):3195-3200 1990; Oren et al. Mol. Cell. Biol 10(8):4007-4015 1990). The family has now grown to about 32 members in humans (Le Naour et al. Proteomics. 6(24):6447-54 2006).

CD9 is a 24 kDa member of this family that is expressed on both hematopoietic and nonhematopoietic cells. Especially high concentrations of CD9 are expressed on the surface of platelets and endothelial cells (Forsyth K D. Immunology 72(2):292-296 1991; Jennings et al. Blood 88(10):624a 1996). CD9 was also recently discovered to be a member of the family of cell surface molecular complexes that include the integrins, other cell surface receptors and other tetraspanins. Several TM4 family members, including CD9, have been found to associate with β1 integrins as well as β2, β3, and β7 integrins (Rubinstein et al. Eur. J. Immunol. 24(12):3005-3013 1994; Nakamura et al. J. Cell Biol. 129(6):1691-1705 1995; Berditchevski et al. Mol. Biol. Cell. 7(2):193-207 1996; Radford et al. Biochem. Biophys. Res. Commun. 222(1):13-28 1996; Hadjiargyrou et al. J Neurochem 67(6):2505-2513 1996; Slupsky et al. Eur J Biochem 244(1):168-175 1997).

Based on cDNA sequence analysis, the TM4SF members are predicted to be single polypeptide chains with four highly hydrophobic putative transmembrane (TM) regions and two extracellular (EC) loops with both the amino and carboxy termini localized intracellularly. Alignment of all tetraspanin amino acid sequences revealed that much of the homology between tetraspanins is confined to the transmembrane domains, which contain a few highly conserved polar amino acids (an asparagine in TM1 and a glutamate or glutamine in TM3 and TM4). These charged residues within the membrane may interact with each other and may be important for the stability of protein assembly, as has been demonstrated for the T cell receptor (Cosson et al. Nature 351(6325):414-416 1991).

There are also conserved hydrophobic residues in all four transmembrane domains; some in TM2 are found in 17/18 tetraspanin sequences. The short region between TM2 and TM3 contains two to three charged residues, including a conserved glutamic acid. These homologies are not shared with other protein families that also have four transmembrane domains, such as the ligand-gated ion channels, connexins, or CD2O/FcERII3.

The conservation between residues observed in the putative transmembrane domains and certain residues in the EC loops, suggests that these proteins perform closely related functions (Maecker et al. FASEB J 11(6):428-442 1997). There is greater sequence divergence in the extracellular loops of tetraspanins, although three cysteines in EC2 are located at defined distances from the TM regions in 16/18 family members. Two of these cysteines occur in a conserved CCG motif located about 50 amino acids past TM3. The third cysteine is often preceded by a glycine and is fixed at 11 amino acids upstream of TM4. A fourth conserved cysteine, frequently found in a PXSC motif, is variably placed in EC2. For some members of this family the use of reducing agents affects their recognition by antibodies indicating that disulfide bonding occurs. Which cysteines are involved is unknown but at least two of the conserved residues in the EC2 are implicated in disulfide bonding (Tomlinson et al. Eur J Immunol 23(1):136-140 1993).

Most of the tetraspanins are modified by N-glycosylation; some are variably glycosylated or acylated, such as CD9 (Seehafer et al. Biochim Biophys Acta 957(3):399-410 1988). The glycosylation patterns between different tetraspanins vary widely. CD9 contains a glycosylation site in EC1 (Boucheix et al. J Biol Chem 266(1):117-122 1991), whereas most other glycosylated tetraspanins contain sites in EC2 (Classon et al. J Exp Med 169(4):1497-1502). Within individual members, however, most glycosylation sites are conserved between species. For example, mouse, rat, primates and cow CD9 all have identical single glycosylation sites, whereas the feline molecule has lost this site altogether.

The expression pattern of some of these proteins have nearly ubiquitous tissue distribution (CD9, CD63, CD81, CD82) whereas others are highly restricted, for example, to lymphoid and myeloid cells (CD53) or mature B cells (CD37). Some members appear to be highly expressed in the immune system; more recently, their expression in the nervous system has also been appreciated. CD9 is transiently expressed in developing spinal motoneurons and other fetal central and peripheral nervous system sites (Tole and Patterson. Dev Dyn 197(2):94-106 1993). It is present in embryonic and fetal hematopoietic tissues (Abe et al. Nippon Ketsueki Gakkai Zasshi. 1989 52(4):712-20 1989; Abe J. Clin Immunol Immunopathol. 1989 51(1):13-21 1989) and is also expressed during B cell development (Boucheix et al. J Biol Chem 266(1):117-122 1991).

Interaction of CD9 with β1 integrins as well as β2, β3, and β7 integrins in particular, suggests that CD9 expression may influence many of the same cellular functions that have been assigned to the integrins. CD9 and other tetraspanins have been reported to participate in the activation, adhesion, and motility of cells as well as in normal and tumor cell growth (Maecker et al. FASEB J 11(6):428-442 1997). While it has been suggested that TM4 family members serve as molecular facilitators (Maecker et al. FASEB J 11(6):428-442 1997), their mode of influence may vary between cells. The transfection of CD9 into poorly motile CD9-negative pre-B cells (Raji) upregulated the motility of these cells across fibronectin and laminin (Shaw et al. 270(41):24092-24099 1995), while transfection of CD9 into nonlymphoid, motile cell lines downregulated their motility to these extracellular matrix components (Ikeyama et al. J. Exp. Med. 177, 1231-1237 1993).

Fibronectin was identified as a potential ligand for CD9 by demonstrating direct binding of fibronectin to immobilized platelet CD9 and to recombinant CD9 (Wilkinson et al. FASEB J. 9:A1500. 23 1995). By using mock- and CD9-transfected CHO cells, Cook et al., compared the adhesion and spreading of these transfected cells to immobilized extracellular matrix components, particularly fibronectin. They showed that: (i) the surface expression of CD9 modifies CHO cell adhesion and spread morphology on fibronectin, (ii) CD9 CHO cell-fibronectin interaction involves primarily the fibronectin segment composed of the HEP2/IIICS binding domain and (iii) CD9 expression down regulates the production of a pericellular fibronectin matrix. These data clearly suggested that ectopic CD9 expression may regulate cell-fibronectin interactions through CD9 binding to specific regions on fibronectin and through modulation of other fibronectin-binding molecules such as α5b1 (Cook et al. Exp Cell Res. 251(2):356-371).

While a number of the associations of tetraspanins are now reasonably well characterized in terms of physical and functional association, others remain controversial, particularly the association of tetraspanins and Fc receptors (FcR). After the demonstration that anti-CD9 antibodies trigger platelet aggregation, it was reported that the antibodies induce association of CD9 with the integrin αIIb/βIII (GPIIb/IIIa; CD4I/CD61) on platelets and that the triggering of platelet aggregation is mediated by GPIIb/IIIa (Slupsky et al. J Biol. Chem. 264(21):12289-12293 1989). In fact, injection of anti-CD9 into monkeys causes lethal thrombocytopenia within 5 minutes of injection, which is prevented by pretreatment of the monkeys with anti-αIIb/β antibodies (Kawakatsu et al. Thromb Res. 70(3):245-254 1993). CD9-mediated platelet activation, like the activation induced by anti-αllb/βIII antibodies, can be blocked by antibodies to FcγRII suggesting that the activation is mediated by FcγRII. Indeed, antibodies to several platelet proteins, including the tetraspanin PETA-3, induce platelet aggregation that is inhibited by Fc receptor blockade.

However, the vast majority of this data describes an indirect relationship because the cellular activation events result from co-ligation of tetraspanins with FcR via the Fc region of intact anti-tetraspanin antibodies. This event is unlikely to be of any significance in normal physiology. The fact that tetraspanins have so frequently been identified as the targets of antibodies which co-ligate FcR is suggestive of a spatial relationship between these molecules. The plethora of reports of tetraspanin-FcR co-ligation has perhaps drawn attention to more physiologically relevant reports which support this relationship, specifically showing proximal co-localization of tetraspanins with FcR by immuno fluorescence and co-immunoprecipitation (Higginbottom et al. 99(4):546-552 2000; Kaji et al. J Immunol 166(5):3256-3265 2001). Such interaction would facilitate cross-talk between FcR and adhesion/signaling molecules in the tetraspanin web which would have clear physiological significance to platelet and immune cell biology. That association of FcR with tetraspanins has important functional effects is implied by the demonstration of tetraspanin-dependent modulation of FcR signaling, both in co-ligation complexes and independently of co-ligation events.

In cancer, clinical studies have reported a link between tetraspanin expression levels and prognosis and/or metastasis. CD9 was initially described on the surface of cells of B-lineage acute lymphoblastic leukemia (Kersey et al. J Exp Med. 153(3):726-31 1981). It is expressed on 90 percent of B-lineage acute leukemias, and on 50 percent of acute myeloid leukemias and B-lineage chronic lymphoid leukemias (Boucheix et al. Leuk Res. 9(5):597-604 1985). In particular, CD9 is a constant marker of acute promyelocytic. The surface presence of CD9 may serve as a prognostic indicator of the metastatic potential of some cancers (Ikeyama et al. J Exp Med. 177(5):1231-1237 1993; Miyake et al. Cancer Res. 55(18):4127-4131 1995). Indeed a high level of the tetraspanins CD9 and CD82/KAI-1 on tumor cells is associated with a favorable prognosis in breast, lung, colon, prostate, and pancreatic cancers. Additionally, a decreased expression level of these molecules is correlated with metastasis in these cancers (Boucheix and Rubinstein. Cell Mol Life Sci. 58(9):1189-1205 2001). CD9 levels were often lower in cells obtained from lymph node metastases than in primary breast cancer tumor cells (Miyake et al. Cancer Res. 55(18):4127-4131 1995). Furthermore, using in vitro and in vivo experimental models, CD9 and CD82 have been shown to act as “metastasis suppressors” whereas CD151 was shown to increase the metastatic potential (Boucheix and Rubinstein. Cell Mol Life Sci. 58(9):1189-1205 2001).

Two recent proteomic studies of tetraspanin web composition in tumor and metastasis has been reported (Andre et al. Proteomics 6(5):1437-1449 2006; Le Naour et al. Mol Cell Proteomics 5(5):845-857 2006). These two reports were both focused on colon cancer using two different cellular models. The models were constituted of cell lines derived from primary colon tumors and metastases from the same patients. The first model was constituted by the cell lines SW480 (primary tumor) and SW620 (lymph node metastasis) (Leibovitz et al. Cancer Res 36(12):4562-4569 1976), available from the American Type Culture Collection (ATCC). The tetraspanin complexes were isolated after immunoaffinity purification and the proteins were identified by MS using LC-ESI-MS/MS and MALDI-FTICR.

The second model was constituted by the three cell lines Isreco1 (IS1, primary tumor), Isreco2 (IS2, liver metastasis), and Isreco3 (IS3, peritoneal metastasis) (Cajot et al. J Biol. Chem. 274(45):31903-31908 1997), established at the ISREC (Institut Suisse d'Etudes Expérimentales sur le Cancer, Swiss). In this study, cells were lysed with the mild detergent Brij97 followed by immunoprecipitation experiments of the CD9-containing complexes. The associated proteins were further eluted using the more stringent detergent Triton X-100, which dissociates tetraspanin-tetraspanin associations. In order to rule out non-specific binding, immunoprecipitation experiments were also performed using an unrelated IgG1 that was treated identically to CD9 mAbs. Protein identification was performed by mass-spectrometry.

A comparative analysis of primary tumor cells and metastases in the two cellular models showed that some proteins were differentially detected. For most of these proteins, the differential expression was confirmed by quantitative methods such as flow cytometry. Important variations in the expression levels of several adhesion molecules were observed, in particular, receptors of the extracellular matrix such as laminin receptors. Interestingly, integrin α6b4 was detected by MS only in CD9-containing complexes from metastases. Immunoprecipitation and Western blotting experiments confirmed that a higher amount of integrin α6b4 was coimmunoprecipitated with CD9 in metastases from both models, despite a similar or lower expression level at the cell surface. Therefore, this suggests a specific recruitment of the integrin α6b4 into tetraspanin-enriched microdomains during tumor progression. In contrast, a significant decrease in other laminin receptors, such as integrin α3b1 and the Ig protein Lu/B-CAM (lutheran/B-cell adhesion molecule), was observed in metastatic cell lines from the two cellular models used as well as on various other metastatic cell lines (Andre et al. Proteomics 6(5):1437-1449 2006).

Another adhesion molecule identified by MS was epithelial cell adhesion molecule (EpCAM). This protein is expressed in many human epithelial tissues and overexpressed in the majority of epithelial carcinomas (Armstrong and Eck. Cancer Biol Ther. 2(4):320-326 2003). Interestingly, it has been demonstrated that EpCAM can associate directly with the tetraspanin CD9. Thus, a substantial colocalization of these two molecules in the normal colon has been observed, whereas the level of co localization was lower in primary tumors and metastases (Le Naour et al. Mol Cell Proteomics 5(5):845-857 2006). Proteomics has also revealed the presence of different membrane proteases (i.e. CD26/dipeptidyl peptidase 4 (DPPIV) expressed only on some metastatic cells) as well as several signaling molecules in tetraspanin-enriched microdomains. These findings may shed a new light on the function of tetraspanins, suggesting that the microdomains may play a role as a platform for enzymatic activities and signal transduction.

In another proteomic study Grønborg et al., demonstrated the use of stable isotope labeling with amino acids in cell culture (SILAC) method to compare the secreted proteins (secretome) from pancreatic cancer-derived cells with that from non-neoplastic pancreatic ductal cells. They identified several proteins that have not been correlated previously with pancreatic cancer including perlecan (HSPG2), CD9 antigen, fibronectin receptor (integrin β1), and a novel cytokine designated as predicted osteoblast protein (FAM3C). Particularly CD9 was identified to be elevated in cancer versus normal by a ratio of 8. Because CD9 was not previously described to be elevated in pancreatic cancer they carried out validation studies by immunohistochemistry (IHC) using pancreatic cancer tissue microarrays (TMAs). CD9 was expressed in robust membranous distribution in 7 of 18 (39 percent) pancreatic cancers on the TMA with no expression seen in adjacent normal pancreatic parenchyma (Grønborg et al. Mol Cell Proteomics. 5(1):157-171). CD9 labeling demonstrated a pattern of apical luminal accentuation similar to the pattern they have reported previously for other secreted proteins in pancreatic cancers such as prostate stem cell antigen and mesothelin (Argani et al. Clin Cancer Res 7(12):3862-3868 2001; Argani et al. Cancer Res. 61(11):4320-4324 2001). In addition, labeling of intraluminal contents was often seen within neoplastic glandular structures, consistent with CD9 secretion.

The protein level quantitation data obtained by the SILAC method was compared with the mRNA data obtained by a DNA microarray experiment. CD9 antigen, which SILAC demonstrated to be differentially over expressed in the pancreatic cancer secretome and was confirmed as being over expressed at the protein level, was down-regulated 2-fold in Panc1 versus HPDE cells based on DNA microarray data. This data reinforce the importance of assessing both the transcriptome and the proteome of human cancers (Grønborg et al. Mol Cell Proteomics. 5(1):157-171).

In another study, the expression of CD9 was examined in primary and metastatic gastric carcinoma tissues. In total, specimens from 78 patients were used for immunohistological staining and specimens from 57 patients were subjected to Northern blotting. CD9 expression was observed at both the message level and the protein level in primary gastric carcinoma tissues, lymph node metastatic tissues, and peritoneal dissemination tissues. CD9 expression was intensified in cancerous areas of gastric cancers in comparison with non cancerous areas in the same patient. When analyzed by the malignancy status based on the clinicopathological diagnosis, there was a tendency that CD9 expression was observed in severe vessel invasion, active lymph node metastasis, and advanced stage. These authors conclude that CD9 expression was rather intensified in gastric cancer tissue in comparison with normal tissues. CD9 expression was more prominent in advanced gastric cancer (Haruko et al. J Surg Res. 117(2):208-215 2004).

The role of CD9 in prostate carcinoma progression was also studied (Wang et al. Clin Cancer Res. 13(8):2354-2361 2007). Reduced or loss of CD9 expression within prostate neoplastic cells was observed in 24 percent of 107 clinically localized primary adenocarcinomas, 85 percent of 60 clinically advanced primary adenocarcinomas, 85 percent of 65 lymph node metastases and 65 percent of 23 bone metastases. This reduction in CD9 expression was associated to alterations of CD9 cDNA not observed in normal tissues. They found that all PC-3 derived cell lines, one PIN and four prostatic adenocarcinomas harbored deletions in their CD9 cDNAs. These deletions removed nucleotides 115 to 487, 190 to 585 or 120 to 619 of the 684 bp CD9 coding sequence. Thus, from the 228 amino acid CD9 protein, amino acids 39 to 163, 64 to 195 or 40 to 207 were eliminated by these deletions. These deletions affected the large extracellular and intracellular domains of the protein. The presence of the PC-3M-LN4 deletion (deletion 64-195) was confirmed on direct sequencing of the mRNA amplification product (without cloning). These deletions were not detected in genomic DNA derived from some of these samples, arguing for the existence of transcriptional CD9 mRNA modifications. Another deletion was detected in the DU145 cell line, whereas an in-frame insertion was present in mRNA derived from PC-3M-Pro4.

Lastly, common missense point mutations were observed in one prostatic carcinoma cell line (PC-3M-LN4), one specimen of PIN, and seven specimens of prostatic adenocarcinoma. Some specimens were harboring more than one missense mutation. Interestingly, CD9 protein expression was not detected in most of these cases (except in one specimen of prostatic adenocarcinoma). A base pair substitution resulting in a new stop codon, located in the second cytoplasmic domain (amino acid 83), was also present in one PIN and in two prostate cancer patients where they did not detect the CD9 protein. Although reduced expression of CD9 protein has been associated with cancer progression in different tumor types, this is the first report implicating CD9 mRNA alterations in CD9 protein inactivation.

The role of CD9 in several cell lines has also been investigated by using anti-CD9 monoclonal antibodies. These experiments demonstrated effects in adhesion and proliferation depending on the cell type and the antibody used. Anti-CD9 antibodies stimulated fibrin clot retraction by fibroblasts (Azzarone et al. J Cell Physiol. 125(3):420-426 1985), induced homotypic adhesion in pre-B lymphocytes (Masellis-Smith et al. J. Immunol. 144(5):1607-1613 1990), inhibited the motility of lung adenocarcinoma cells (Miyake et al. J Exp Med. 174(6):1347-1354 1991), augmented the adherence of neutrophils to endothelial cells (Forsyth K D. Immunology 72(2):292-296 1991) and elicited phosphatidylinositol turnover, phosphatidylinositol biosynthesis and protein-tyrosine phosphorylation in human platelets (Yatomi et al. FEBS Lett. 322(3):285-290 1993). One anti-CD9 monoclonal antibody, B2C11, promoted adhesion of a number of Schwann cell lines, PC12 cells and primary rat Schwann cells (Hadjiargyrou and Patterson. J. Neurosci. 15(1 Pt 2):574-583 1995). In addition, this antibody also stimulated proliferation of one of the Schwann cell lines. In another article the same group further demonstrated that another anti-CD9 monoclonal antibody, SMRA1, enhanced motility and migration in primary Schwann cells which is correlated with an increase in cytosolic calcium and phosphoproteins (Anton et al. J. Neurosci. 15(1 Pt 2):584-95 1994). However, none of these antibodies have been reported to have been tested in an in vivo model of human cancer.

Finally, a recent report showed that ectopic expression of CD9 in colon carcinoma cells resulted in enhanced integrin-dependent adhesion and inhibition of cell growth. Consistent with these effects, treatment of these cells with anti-CD9 specific antibodies resulted in (i) increased β1 integrin-mediated cell adhesion through a mechanism involving clustering of integrin molecules rather than altered affinity; (ii) induction of morphological changes characterized by the acquisition of an elongated cell phenotype; (iii) inhibition of cell proliferation with no significant effect on cell survival; (iv) increased expression of membrane TNF-α and finally (v) inhibition of the in vivo tumorigenic capacity in nude mice. In addition, through the use of selective blockers of TNF-α, they have demonstrated that this cytokine partly mediates the anti-proliferative effects of CD9 (Ovalle et al. Int J Cancer. [Epub ahead of print] 2007). The two anti-CD9 antibodies tested in vivo, VJ1/20 and PAINS-13, were tested in a prophylactic type xenograft model whereas the anti-CD9 antibodies disclosed herein have demonstrated efficacy in both prophylactic and, more clinically relevant, established xenograft models of human cancer. In addition, unlike VJ1/20 or PAINS-13, the anti-CD9 antibodies disclosed herein have demonstrated in vivo efficacy in more than one cancer xenograft model.

Monoclonal Antibodies as Cancer Therapy: Each individual who presents with cancer is unique and has a cancer that is as different from other cancers as that person's identity. Despite this, current therapy treats all patients with the same type of cancer, at the same stage, in the same way. At least 30 percent of these patients will fail the first line therapy, thus leading to further rounds of treatment and the increased probability of treatment failure, metastases, and ultimately, death. A superior approach to treatment would be the customization of therapy for the particular individual. The only current therapy which lends itself to customization is surgery. Chemotherapy and radiation treatment cannot be tailored to the patient, and surgery by itself, in most cases is inadequate for producing cures.

With the advent of monoclonal antibodies, the possibility of developing methods for customized therapy became more realistic since each antibody can be directed to a single epitope. Furthermore, it is possible to produce a combination of antibodies that are directed to the constellation of epitopes that uniquely define a particular individual's tumor.

Having recognized that a significant difference between cancerous and normal cells is that cancerous cells contain antigens that are specific to transformed cells, the scientific community has long held that monoclonal antibodies can be designed to specifically target transformed cells by binding specifically to these cancer antigens; thus giving rise to the belief that monoclonal antibodies can serve as “Magic Bullets” to eliminate cancer cells. However, it is now widely recognized that no single monoclonal antibody can serve in all instances of cancer, and that monoclonal antibodies can be deployed, as a class, as targeted cancer treatments. Monoclonal antibodies isolated in accordance with the teachings of the instantly disclosed invention have been shown to modify the cancerous disease process in a manner which is beneficial to the patient, for example by reducing the tumor burden, and will variously be referred to herein as cancerous disease modifying antibodies (CDMAB) or “anti-cancer” antibodies.

At the present time, the cancer patient usually has few options of treatment. The regimented approach to cancer therapy has produced improvements in global survival and morbidity rates. However, to the particular individual, these improved statistics do not necessarily correlate with an improvement in their personal situation.

Thus, if a methodology was put forth which enabled the practitioner to treat each tumor independently of other patients in the same cohort, this would permit the unique approach of tailoring therapy to just that one person. Such a course of therapy would, ideally, increase the rate of cures, and produce better outcomes, thereby satisfying a long-felt need.

Historically, the use of polyclonal antibodies has been used with limited success in the treatment of human cancers. Lymphomas and leukemias have been treated with human plasma, but there were few prolonged remission or responses. Furthermore, there was a lack of reproducibility and there was no additional benefit compared to chemotherapy. Solid tumors such as breast cancers, melanomas and renal cell carcinomas have also been treated with human blood, chimpanzee serum, human plasma and horse serum with correspondingly unpredictable and ineffective results.

There have been many clinical trials of monoclonal antibodies for solid tumors. In the 1980s there were at least four clinical trials for human breast cancer which produced only one responder from at least 47 patients using antibodies against specific antigens or based on tissue selectivity. It was not until 1998 that there was a successful clinical trial using a humanized anti-Her2/neu antibody (Herceptin®) in combination with CISPLATIN. In this trial 37 patients were assessed for responses of which about a quarter had a partial response rate and an additional quarter had minor or stable disease progression. The median time to progression among the responders was 8.4 months with median response duration of 5.3 months.

Herceptin® was approved in 1998 for first line use in combination with Taxol®. Clinical study results showed an increase in the median time to disease progression for those who received antibody therapy plus Taxol® (6.9 months) in comparison to the group that received Taxol® alone (3.0 months). There was also a slight increase in median survival; 22 versus 18 months for the Herceptin® plus Taxol® treatment arm versus the Taxol® treatment alone arm. In addition, there was an increase in the number of both complete (8 versus 2 percent) and partial responders (34 versus 15 percent) in the antibody plus Taxol® combination group in comparison to Taxol® alone. However, treatment with Herceptin® and Taxol® led to a higher incidence of cardiotoxicity in comparison to Taxol® treatment alone (13 versus 1 percent respectively). Also, Herceptin® therapy was only effective for patients who over express (as determined through immunohistochemistry (IHC) analysis) the human epidermal growth factor receptor 2 (Her2/neu), a receptor, which currently has no known function or biologically important ligand; approximately 25 percent of patients who have metastatic breast cancer. Therefore, there is still a large unmet need for patients with breast cancer. Even those who can benefit from Herceptin® treatment would still require chemotherapy and consequently would still have to deal with, at least to some degree, the side effects of this kind of treatment.

The clinical trials investigating colorectal cancer involve antibodies against both glycoprotein and glycolipid targets. Antibodies such as 17-1A, which has some specificity for adenocarcinomas, has undergone Phase 2 clinical trials in over 60 patients with only 1 patient having a partial response. In other trials, use of 17-1A produced only 1 complete response and 2 minor responses among 52 patients in protocols using additional cyclophosphamide. To date, Phase III clinical trials of 17-1A have not demonstrated improved efficacy as adjuvant therapy for stage III colon cancer. The use of a humanized murine monoclonal antibody initially approved for imaging also did not produce tumor regression.

Only recently have there been any positive results from colorectal cancer clinical studies with the use of monoclonal antibodies. In 2004, ERBITUX® was approved for the second line treatment of patients with EGFR-expressing metastatic colorectal cancer who are refractory to irinotecan-based chemotherapy. Results from both a two-arm Phase II clinical study and a single arm study showed that ERBITUX® in combination with irinotecan had a response rate of 23 and 15 percent respectively with a median time to disease progression of 4.1 and 6.5 months respectively. Results from the same two-arm Phase II clinical study and another single arm study showed that treatment with ERBITUX® alone resulted in an 11 and 9 percent response rate respectively with a median time to disease progression of 1.5 and 4.2 months respectively.

Consequently in both Switzerland and the United States, ERBITUX® treatment in combination with irinotecan, and in the United States, ERBITUX® treatment alone, has been approved as a second line treatment of colon cancer patients who have failed first line irinotecan therapy. Therefore, like Herceptin®, treatment in Switzerland is only approved as a combination of monoclonal antibody and chemotherapy. In addition, treatment in both Switzerland and the US is only approved for patients as a second line therapy. Also, in 2004, AVASTIN® was approved for use in combination with intravenous 5-fluorouracil-based chemotherapy as a first line treatment of metastatic colorectal cancer. Phase III clinical study results demonstrated a prolongation in the median survival of patients treated with AVASTIN® plus 5-fluorouracil compared to patients treated with 5-fluourouracil alone (20 months versus 16 months respectively). However, again like Herceptin® and ERBITUX®, treatment is only approved as a combination of monoclonal antibody and chemotherapy.

There also continues to be poor results for lung, brain, ovarian, pancreatic, prostate, and stomach cancer. The most promising recent results for non-small cell lung cancer came from a Phase II clinical trial where treatment involved a monoclonal antibody (SGN-15; dox-BR96, anti-Sialyl-LeX) conjugated to the cell-killing drug doxorubicin in combination with the chemotherapeutic agent TAXOTERE®. TAXOTERE® is the only FDA approved chemotherapy for the second line treatment of lung cancer. Initial data indicate an improved overall survival compared to TAXOTERE® alone. Out of the 62 patients who were recruited for the study, two-thirds received SGN-15 in combination with TAXOTERE® while the remaining one-third received TAXOTERE® alone. For the patients receiving SGN-15 in combination with TAXOTERE®, median overall survival was 7.3 months in comparison to 5.9 months for patients receiving TAXOTERE® alone. Overall survival at 1 year and 18 months was 29 and 18 percent respectively for patients receiving SNG-15 plus TAXOTERE® compared to 24 and 8 percent respectively for patients receiving TAXOTERE® alone. Further clinical trials are planned.

Preclinically, there has been some limited success in the use of monoclonal antibodies for melanoma. Very few of these antibodies have reached clinical trials and to date none have been approved or demonstrated favorable results in Phase III clinical trials.

The discovery of new drugs to treat disease is hindered by the lack of identification of relevant targets among the products of 30,000 known genes that could contribute to disease pathogenesis. In oncology research, potential drug targets are often selected simply due to the fact that they are over-expressed in tumor cells. Targets thus identified are then screened for interaction with a multitude of compounds. In the case of potential antibody therapies, these candidate compounds are usually derived from traditional methods of monoclonal antibody generation according to the fundamental principles laid down by Kohler and Milstein (1975, Nature, 256, 495-497, Kohler and Milstein). Spleen cells are collected from mice immunized with antigen (e.g. whole cells, cell fractions, purified antigen) and fused with immortalized hybridoma partners. The resulting hybridomas are screened and selected for secretion of antibodies which bind most avidly to the target. Many therapeutic and diagnostic antibodies directed against cancer cells, including Herceptin® and RITUXIMAB, have been produced using these methods and selected on the basis of their affinity. The flaws in this strategy are two-fold. Firstly, the choice of appropriate targets for therapeutic or diagnostic antibody binding is limited by the paucity of knowledge surrounding tissue specific carcinogenic processes and the resulting simplistic methods, such as selection by overexpression, by which these targets are identified. Secondly, the assumption that the drug molecule that binds to the receptor with the greatest affinity usually has the highest probability for initiating or inhibiting a signal may not always be the case.

Despite some progress with the treatment of breast and colon cancer, the identification and development of efficacious antibody therapies, either as single agents or co-treatments, have been inadequate for all types of cancer.

### Prior Patents:

U.S. Pat. No. 5,858,358 and U.S. application Ser. No. 09/183,055 both disclose the monoclonal antibody ES5.2D8 and that it recognizes CD9.

U.S. application Ser. No. 10/619,323 discloses the role of CD9 in adhesion and proliferation and the region of CD9 that is recognized by monoclonal antibody mAb7. The application also discloses that the treatment of mAb7 to coronary smooth muscle cells decreases cell proliferation in vitro.

U.S. Pat. No. 5,750,102 discloses a process wherein cells from a patient's tumor are transfected with MHC genes which may be cloned from cells or tissue from the patient. These transfected cells are then used to vaccinate the patient.

U.S. Pat. No. 4,861,581 discloses a process comprising the steps of obtaining monoclonal antibodies that are specific to an internal cellular component of neoplastic and normal cells of the mammal but not to external components, labeling the monoclonal antibody, contacting the labeled antibody with tissue of a mammal that has received therapy to kill neoplastic cells, and determining the effectiveness of therapy by measuring the binding of the labeled antibody to the internal cellular component of the degenerating neoplastic cells. In preparing antibodies directed to human intracellular antigens, the patentee recognizes that malignant cells represent a convenient source of such antigens.

U.S. Pat. No. 5,171,665 provides a novel antibody and method for its production. Specifically, the patent teaches formation of a monoclonal antibody which has the property of binding strongly to a protein antigen associated with human tumors, e.g. those of the colon and lung, while binding to normal cells to a much lesser degree.

U.S. Pat. No. 5,484,596 provides a method of cancer therapy comprising surgically removing tumor tissue from a human cancer patient, treating the tumor tissue to obtain tumor cells, irradiating the tumor cells to be viable but non-tumorigenic, and using these cells to prepare a vaccine for the patient capable of inhibiting recurrence of the primary tumor while simultaneously inhibiting metastases. The patent teaches the development of monoclonal antibodies which are reactive with surface antigens of tumor cells. As set forth at col. 4, lines 45 et seq., the patentees utilize autochthonous tumor cells in the development of monoclonal antibodies expressing active specific immunotherapy in human neoplasia.

U.S. Pat. No. 5,693,763 teaches a glycoprotein antigen characteristic of human carcinomas and not dependent upon the epithelial tissue of origin.

U.S. Pat. No. 5,783,186 is drawn to Anti-Her2 antibodies which induce apoptosis in Her2 expressing cells, hybridoma cell lines producing the antibodies, methods of treating cancer using the antibodies and pharmaceutical compositions including said antibodies.

U.S. Pat. No. 5,849,876 describes new hybridoma cell lines for the production of monoclonal antibodies to mucin antigens purified from tumor and non-tumor tissue sources.

U.S. Pat. No. 5,869,268 is drawn to a method for generating a human lymphocyte producing an antibody specific to a desired antigen, a method for producing a monoclonal antibody, as well as monoclonal antibodies produced by the method. The patent is particularly drawn to the production of an anti-HD human monoclonal antibody useful for the diagnosis and treatment of cancers.

U.S. Pat. No. 5,869,045 relates to antibodies, antibody fragments, antibody conjugates and single-chain immunotoxins reactive with human carcinoma cells. The mechanism by which these antibodies function is two-fold, in that the molecules are reactive with cell membrane antigens present on the surface of human carcinomas, and further in that the antibodies have the ability to internalize within the carcinoma cells, subsequent to binding, making them especially useful for forming antibody-drug and antibody-toxin conjugates. In their unmodified form the antibodies also manifest cytotoxic properties at specific concentrations.

U.S. Pat. No. 5,780,033 discloses the use of autoantibodies for tumor therapy and prophylaxis. However, this antibody is an antinuclear autoantibody from an aged mammal. In this case, the autoantibody is said to be one type of natural antibody found in the immune system. Because the autoantibody comes from “an aged mammal”, there is no requirement that the autoantibody actually comes from the patient being treated. In addition the patent discloses natural and monoclonal antinuclear autoantibody from an aged mammal, and a hybridoma cell line producing a monoclonal antinuclear autoantibody.

### SUMMARY OF THE INVENTION

This application utilizes methodology for producing patient specific anti-cancer antibodies taught in the U.S. Pat. No. 6,180,357 patent for isolating hybridoma cell lines which encode for cancerous disease modifying monoclonal antibodies. These antibodies can be made specifically for one tumor and thus make possible the customization of cancer therapy. Within the context of this application, anti-cancer antibodies having either cell-killing (cytotoxic) or cell-growth inhibiting (cytostatic) properties will hereafter be referred to as cytotoxic. These antibodies can be used in aid of staging and diagnosis of a cancer, and can be used to treat tumor metastases. These antibodies can also be used for the prevention of cancer by way of prophylactic treatment. Unlike antibodies generated according to traditional drug discovery paradigms, antibodies generated in this way may target molecules and pathways not previously shown to be integral to the growth and/or survival of malignant tissue. Furthermore, the binding affinities of these antibodies are suited to requirements for initiation of the cytotoxic events that may not be amenable to stronger affinity interactions. Also, it is within the purview of this invention to conjugate standard chemotherapeutic modalities, e.g. radionuclides, with the CDMAB of the instant invention, thereby focusing the use of said chemotherapeutics. The CDMAB can also be conjugated to toxins, cytotoxic moieties, enzymes e.g. biotin conjugated enzymes, cytokines, interferons, target or reporter moieties or hematogenous cells, thereby forming an antibody conjugate. The CDMAB can be used alone or in combination with one or more CDMAB/chemotherapeutic agents.

The prospect of individualized anti-cancer treatment will bring about a change in the way a patient is managed. A likely clinical scenario is that a tumor sample is obtained at the time of presentation, and banked. From this sample, the tumor can be typed from a panel of pre-existing cancerous disease modifying antibodies. The patient will be conventionally staged but the available antibodies can be of use in further staging the patient. The patient can be treated immediately with the existing antibodies, and a panel of antibodies specific to the tumor can be produced either using the methods outlined herein or through the use of phage display libraries in conjunction with the screening methods herein disclosed. All the antibodies generated will be added to the library of anti-cancer antibodies since there is a possibility that other tumors can bear some of the same epitopes as the one that is being treated. The antibodies produced according to this method may be useful to treat cancerous disease in any number of patients who have cancers that bind to these antibodies.

In addition to anti-cancer antibodies, the patient can elect to receive the currently recommended therapies as part of a multi-modal regimen of treatment. The fact that the antibodies isolated via the present methodology are relatively non-toxic to non-cancerous cells allows for combinations of antibodies at high doses to be used, either alone, or in conjunction with conventional therapy. The high therapeutic index will also permit re-treatment on a short time scale that should decrease the likelihood of emergence of treatment resistant cells.

If the patient is refractory to the initial course of therapy or metastases develop, the process of generating specific antibodies to the tumor can be repeated for re-treatment. Furthermore, the anti-cancer antibodies can be conjugated to red blood cells obtained from that patient and re-infused for treatment of metastases. There have been few effective treatments for metastatic cancer and metastases usually portend a poor outcome resulting in death. However, metastatic cancers are usually well vascularized and the delivery of anti-cancer antibodies by red blood cells can have the effect of concentrating the antibodies at the site of the tumor. Even prior to metastases, most cancer cells are dependent on the host's blood supply for their survival and an anti-cancer antibody conjugated to red blood cells can be effective against in situ tumors as well. Alternatively, the antibodies may be conjugated to other hematogenous cells, e.g. lymphocytes, macrophages, monocytes, natural killer cells, etc.

There are five classes of antibodies and each is associated with a function that is conferred by its heavy chain. It is generally thought that cancer cell killing by naked antibodies are mediated either through antibody dependent cellular cytotoxicity (ADCC) or complement dependent cytotoxicity (CDC). For example murine IgM and IgG2a antibodies can activate human complement by binding the C-1 component of the complement system thereby activating the classical pathway of complement activation which can lead to tumor lysis. For human antibodies the most effective complement activating antibodies are generally IgM and IgG1. Murine antibodies of the IgG2a and IgG3 isotype are effective at recruiting cytotoxic cells that have Fc receptors which will lead to cell killing by monocytes, macrophages, granulocytes and certain lymphocytes. Human antibodies of both the IgG1 and IgG3 isotype mediate ADCC.

The cytotoxicity mediated through the Fc region requires the presence of effector cells, their corresponding receptors, or proteins e.g. NK cells, T-cells and complement. In the absence of these effector mechanisms, the Fc portion of an antibody is inert. The Fc portion of an antibody may confer properties that affect the pharmacokinetics of an antibody in vivo, but in vitro this is not operative.

The cytotoxicity assays under which we test the antibodies do not have any of the effector mechanisms present, and are carried out in vitro. These assays do not have effector cells (NK, Macrophages, or T-cells) or complement present. Since these assays are completely defined by what is added together, each component can be characterized. The assays used herein contain only target cells, media and sera. The target cells do not have effector functions since they are cancer cells or fibroblasts. Without exogenous cells which have effector function properties there is no cellular elements that have this function. The media does not contain complement or any cells. The sera used to support the growth of the target cells do not have complement activity as disclosed by the vendors. Furthermore, in our own labs we have verified the absence of complement activity in the sera used. Therefore, our work evidences the fact that the effects of the antibodies are due entirely to the effects of the antigen binding which is mediated through the Fab. Effectively, the target cells are seeing and interacting with only the Fab, since they do not have receptors for the Fc. Although, the hybridoma is secreting complete immunoglobulin which was tested with the target cells, the only part of the immunoglobulin that interacts with the cells are the Fab, which act as antigen binding fragments.

With respect to the instantly claimed antibodies and antigen binding fragments, the application, as filed, has demonstrated cellular cytotoxicity as evidenced by the data in FIG. 1. As pointed out above, and as herein confirmed via objective evidence, this effect was entirely due to binding by the Fab to the tumor cells.

Ample evidence exists in the art of antibodies mediating cytotoxicity due to direct binding of the antibody to the target antigen independent of effector mechanisms recruited by the Fc. The best evidence for this is in vitro experiments which do not have supplemental cells, or complement (to formally exclude those mechanisms). These types of experiments have been carried out with complete immunoglobulin, or with antigen binding fragments such as F(ab′)₂ fragments. In these types of experiments, antibodies or antigen binding fragments can directly induce apoptosis of target cells such as in the case of anti-Her2 and anti-EGFR antibodies, both of which have antibodies that are approved by the US FDA for marketing in cancer therapy.

Another possible mechanism of antibody mediated cancer killing may be through the use of antibodies that function to catalyze the hydrolysis of various chemical bonds in the cell membrane and its associated glycoproteins or glycolipids, so-called catalytic antibodies.

There are three additional mechanisms of antibody-mediated cancer cell killing. The first is the use of antibodies as a vaccine to induce the body to produce an immune response against the putative antigen that resides on the cancer cell. The second is the use of antibodies to target growth receptors and interfere with their function or to down regulate that receptor so that its function is effectively lost. The third is the effect of such antibodies on direct ligation of cell surface moieties that may lead to direct cell death, such as ligation of death receptors such as TRAIL R1 or TRAIL R2, or integrin molecules such as alpha V beta 3 and the like.

The clinical utility of a cancer drug is based on the benefit of the drug under an acceptable risk profile to the patient. In cancer therapy survival has generally been the most sought after benefit, however there are a number of other well-recognized benefits in addition to prolonging life. These other benefits, where treatment does not adversely affect survival, include symptom palliation, protection against adverse events, prolongation in time to recurrence or disease-free survival, and prolongation in time to progression. These criteria are generally accepted and regulatory bodies such as the U.S. Food and Drug Administration (F.D.A.) approve drugs that produce these benefits (Hirschfeld et al. Critical Reviews in Oncology/Hematology 42:137-143 2002). In addition to these criteria it is well recognized that there are other endpoints that may presage these types of benefits. In part, the accelerated approval process granted by the U.S. F.D.A. acknowledges that there are surrogates that will likely predict patient benefit. As of year-end 2003, there have been sixteen drugs approved under this process, and of these, four have gone on to full approval, i.e., follow-up studies have demonstrated direct patient benefit as predicted by surrogate endpoints. One important endpoint for determining drug effects in solid tumors is the assessment of tumor burden by measuring response to treatment (Therasse et al. Journal of the National Cancer Institute 92(3):205-216 2000). The clinical criteria (RECIST criteria) for such evaluation have been promulgated by Response Evaluation Criteria in Solid Tumors Working Group, a group of international experts in cancer. Drugs with a demonstrated effect on tumor burden, as shown by objective responses according to RECIST criteria, in comparison to the appropriate control group tend to, ultimately, produce direct patient benefit. In the pre-clinical setting tumor burden is generally more straightforward to assess and document. In that pre-clinical studies can be translated to the clinical setting, drugs that produce prolonged survival in pre-clinical models have the greatest anticipated clinical utility. Analogous to producing positive responses to clinical treatment, drugs that reduce tumor burden in the pre-clinical setting may also have significant direct impact on the disease. Although prolongation of survival is the most sought after clinical outcome from cancer drug treatment, there are other benefits that have clinical utility and it is clear that tumor burden reduction, which may correlate to a delay in disease progression, extended survival or both, can also lead to direct benefits and have clinical impact (Eckhardt et al. Developmental Therapeutics: Successes and Failures of Clinical Trial Designs of Targeted Compounds; ASCO Educational Book, 39^{th} Annual Meeting, 2003, pages 209-219).

The present invention describes the development and use of AR40A746.2.3 identified by its effect in a cytotoxic assay and in an animal model of human cancer. This invention describes reagents that bind specifically to an epitope or epitopes present on the target molecule, and that also have in vitro cytotoxic properties, as a naked antibody, against malignant tumor cells but not normal cells, and which also directly mediate, as a naked antibody, inhibition of tumor growth. A further advance is of the use of anti-cancer antibodies such as this to target tumors expressing cognate antigen markers to achieve tumor growth inhibition, and other positive endpoints of cancer treatment.

In all, this invention teaches the use of the AR40A746.2.3 antigen as a target for a therapeutic agent, that when administered can reduce the tumor burden of a cancer expressing the antigen in a mammal. This invention also teaches the use of CDMAB (AR40A746.2.3), and its derivatives, and antigen binding fragments thereof, and cytotoxicity inducing ligands thereof, to target their antigen to reduce the tumor burden of a cancer expressing the antigen in a mammal. Furthermore, this invention also teaches the use of detecting the AR40A746.2.3 antigen in cancerous cells that can be useful for the diagnosis, prediction of therapy, and prognosis of mammals bearing tumors that express this antigen.

Accordingly, it is an objective of the invention to utilize a method for producing cancerous disease modifying antibodies (CDMAB) raised against cancerous cells derived from a particular individual, or one or more particular cancer cell lines, which CDMAB are cytotoxic with respect to cancer cells while simultaneously being relatively non-toxic to non-cancerous cells, in order to isolate hybridoma cell lines and the corresponding isolated monoclonal antibodies and antigen binding fragments thereof for which said hybridoma cell lines are encoded.

It is an additional objective of the invention to teach cancerous disease modifying antibodies, ligands and antigen binding fragments thereof.

It is a further objective of the instant invention to produce cancerous disease modifying antibodies whose cytotoxicity is mediated through antibody dependent cellular toxicity.

It is yet an additional objective of the instant invention to produce cancerous disease modifying antibodies whose cytotoxicity is mediated through complement dependent cellular toxicity.

It is still a further objective of the instant invention to produce cancerous disease modifying antibodies whose cytotoxicity is a function of their ability to catalyze hydrolysis of cellular chemical bonds.

A still further objective of the instant invention is to produce cancerous disease modifying antibodies which are useful for in a binding assay for diagnosis, prognosis, and monitoring of cancer.

Other objects and advantages of this invention will become apparent from the following description wherein are set forth, by way of illustration and example, certain embodiments of this invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 compares the percentage cytotoxicity and binding levels of the hybridoma supernatants against cell lines PC-3, LnCap and CCD-27sk.

FIG. 2 represents binding of AR40A746.2.3 to cancer and normal cell lines. The data is tabulated to present the mean fluorescence intensity as a fold increase above isotype control.

FIG. 3 includes representative FACS histograms of AR40A746.2.3 and anti-EGFR antibodies directed against several cancer and non-cancer cell lines.

FIG. 4 demonstrates the effect of AR40A746.2.3 on tumor growth in a prophylactic BxPC-3 pancreatic cancer model. The vertical dashed lines indicate the period during which the antibody was administered. Data points represent the mean+/−SEM.

FIG. 5 demonstrates the effect of AR40A746.2.3 on body weight in a prophylactic BxPC-3 pancreatic cancer model. Data points represent the mean+/−SEM.

FIG. 6 demonstrates the effect of AR40A746.2.3 on tumor growth in an established BxPC-3 pancreatic cancer model. The vertical dashed lines indicate the period during which the antibody was administered. Data points represent the mean+/−SEM.

FIG. 7 demonstrates the effect of AR40A746.2.3 on body weight in an established BxPC-3 pancreatic cancer model. Data points represent the mean+/−SEM.

FIG. 8 demonstrates the effect of AR40A746.2.3 on tumor growth in an established MDA-MB-231 breast cancer model. The vertical dashed lines indicate the period during which the antibody was administered. Data points represent the mean+/−SEM.

FIG. 9 demonstrates the effect of AR40A746.2.3 on body weight in an established MDA-MB-231 breast cancer model. Data points represent the mean+/−SEM.

FIG. 10 demonstrates the effect of AR40A746.2.3 on tumor growth in a dose-dependent manner in a BxPC-3 pancreatic cancer model. The vertical dashed lines indicate the period during which the antibody was administered. Data points represent the mean+/−SEM.

FIG. 11 demonstrates the effect of various doses of AR40A746.2.3 on body weight in a BxPC-3 pancreatic cancer model. Data points represent the mean+/−SEM.

FIG. 12 demonstrates the effect of AR40A746.2.3 and AR40A746.2.3 F(ab′)₂ on tumor growth in an established human BxPC-3 pancreatic cancer model. The vertical dashed lines indicate the period during which the antibody was intraperitoneally administered. Data points represent the mean+/−SEM.

FIG. 13 demonstrates the effect of AR40A746.2.3 and AR40A746.2.3 F(ab′)₂ on mouse body weight in an established BxPC-3 pancreatic cancer model. Data points represent the mean+/−SEM.

FIG. 14 demonstrates the effect of AR40A746.2.3 and 80 mg/kg gemcitabine alone and in combination on median tumor growth in an established human pancreatic (BxPC-3) cancer model.

FIG. 15 demonstrates the effect of AR40A746.2.3 and 160 mg/kg gemcitabine alone and in combination on median tumor growth in an established human pancreatic (BxPC-3) cancer model.

FIG. 16 demonstrates the effect of AR40A746.2.3 and 80 mg/kg gemcitabine alone and in combination on mouse survival in an established human pancreatic (BxPC-3) cancer model.

FIG. 17 demonstrates the effect of AR40A746.2.3 and 160 mg/kg gemcitabine alone and in combination on mouse survival in an established human pancreatic (BxPC-3) cancer model.

FIG. 18 demonstrates the effect of AR40A746.2.3 and 80 mg/kg gemcitabine alone and in combination on mouse body weight in an established BxPC-3 pancreatic cancer model.

FIG. 19 demonstrates the effect of AR40A746.2.3 and 160 mg/kg gemcitabine alone and in combination on mouse body weight in an established BxPC-3 pancreatic cancer model.

FIG. 20 demonstrates the effect of AR40A746.2.3 on tumor growth in a prophylactic human MDA-MB-231 breast adenocarcinoma model. The vertical dashed lines indicate the period during which the antibody was intraperitoneally administered. Data points represent the mean+/−SEM.

FIG. 21 demonstrates the effect of AR40A746.2.3 on mouse body weight in a prophylactic MDA-MB-231 breast adenocarcinoma model. Data points represent the mean+/−SEM.

FIGS. 22A-22B tabulate an IHC comparison of AR40A746.2.3 on various human normal tissue sections from a tissue micro array.

FIGS. 23A-23C tabulate an IHC comparison of AR40A746.2.3 on various human normal and tumor tissue sections from two human tissue micro arrays.

FIG. 24. Representative micrographs showing the binding pattern obtained with AR40A746.2.3 on human kidney transitional cell carcinoma tumor tissue (A) or normal human kidney tissue (B) and on human esophageal squamous cell carcinoma tumor tissue (C) or normal human esophagus tissue (D) from human tumor and normal tissue micro arrays. Magnification is 200×.

FIG. 25 tabulates an IHC comparison of AR40A746.2.3 on various human pancreatic tumor tissue sections from a tissue micro array.

FIG. 26. Representative micrographs showing the binding pattern obtained with AR40A746.2.3 on pancreatic adenocarcinoma (A) or normal human pancreatic tissue (B) from a human pancreatic tumor and normal tissue micro array. Magnification is 200×.

FIG. 27 tabulates an IHC comparison of AR40A746.2.3 on various species normal tissue sections from multiple tissue micro arrays.

FIG. 28. Representative micrographs showing the binding pattern obtained with AR40A746.2.3 on normal spleen tissue from human (A), cynomolgus monkey (B), rhesus monkey (C) or rabbit (D) from various species micro arrays. AR40A746.2.3 bound to lymphocytes and endothelium of splenic sinusoids of human, cynomolgus, rhesus and rabbit. Magnification is 200×.

FIG. 29. SDS-PAGE of immunoprecipitation products. Lane 1 contains the AR40A746.2.3 immunoprecipitated material, lane 2 contains the IgG1 isotype control (clone 1B7.11) immunoprecipitated material and lane 3 contains molecular weight standard. The 25 kDa band immunoprecipitated by AR40A746.2.3 is indicated by the arrow.

FIG. 30. Overview of the calibrated spectra of AR40A746.2.3 immunoprecipitate and IgG1 (clone 1B7.11) tryptic digests. Peaks specific to the AR40A746.2.3 immunoprecipitate digest are labeled with molecular weights.

FIG. 31. Western blots of proteins probed with AR40A746.2.3 (Panel A), anti-CD9 (clone MEM-61; Panel B) and IgG1 isotype control (clone 1B7.11; Panel C). Lane 1: AR40A746.2.3 immunoprecipitate, lane 3: anti-CD9 (clone MEM-61) immunoprecipitate, lane 4: IgG1 isotype control (clone 1B7.11) immunoprecipitate, lane 5: BxPC-3 lysate (20 micrograms) and lane 6: molecular weight marker (molecular weights in kDa are listed beside each band).

FIG. 32. List of primers (SEQ ID NOS 9-17, respectively in order of appearance) used for the PCR amplification of AR40A746.2.3 heavy and light chain.

FIG. 33. Protein sequence of the heavy (SEQ ID NO: 7) and light chain (SEQ ID NO: 8) of AR40A746.2.3. CDR regions are underlined and highlighted in blue (SEQ ID NOS 4-5 and 1-3, respectively in order of appearance).

FIG. 34. List of kinases whose phosphorylation is affected by treatment of BxPC-3 cells treated with AR40A746.2.3 followed by serum and supplement stimulation.

FIG. 35. List of RTKs whose phosphorylation is affected by treatment of BxPC-3 cells treated with AR40A746.2.3 followed by serum and supplement stimulation.

FIG. 36 represents the total apoptotic effects of the murine AR40A746.2.3 antibody on BxPC-3 pancreatic cell line at 24 and 40 hours obtained by Annexin-V staining experiments.

### DETAILED DESCRIPTION OF THE INVENTION

In general, the following words or phrases have the indicated definition when used in the summary, description, examples, and claims.

The term “antibody” is used in the broadest sense and specifically covers, for example, single monoclonal antibodies (including agonist, antagonist, and neutralizing antibodies, de-immunized, murine, chimeric or humanized antibodies), antibody compositions with polyepitopic specificity, single-chain antibodies, diabodies, triabodies, immunoconjugates and antibody fragments (see below).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma (murine or human) method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

“Antibody fragments” comprise a portion of an intact antibody, preferably comprising the antigen-binding or variable region thereof. Examples of antibody fragments include less than full length antibodies, Fab, Fab′, F(ab′)₂, and Fv fragments; diabodies; linear antibodies; single-chain antibody molecules; single-chain antibodies, single domain antibody molecules, fusion proteins, recombinant proteins and multispecific antibodies formed from antibody fragment(s).

An “intact” antibody is one which comprises an antigen-binding variable region as well as a light chain constant domain (C_{L}) and heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

Depending on the amino acid sequence of the constant domain of their heavy chains, intact antibodies can be assigned to different “classes”. There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Antibody “effector functions” refer to those biological activities attributable to the Fc region (a native sequence Fe region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and Fcy RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., Eur. J. Immunol. 24:2429 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 2632 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. The Fab fragment also contains the constant domain of the light chain and the first constant domain (CH I) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “triabodies” or “trivalent trimers” refers to the combination of three single chain antibodies. Triabodies are constructed with the amino acid terminus of a V_{L} or V_{H} domain, i.e., without any linker sequence. A triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. Isolated antibody includes the antibody in situ within recombinant cells since at least one component of the antibody's natural environment will not be present. Ordinarily, however, isolated antibody will be prepared by at least one purification step.

An antibody “which binds” an antigen of interest, e.g. CD9 antigen, is one capable of binding that antigen with sufficient affinity such that the antibody is useful as a therapeutic or diagnostic agent in targeting a cell expressing the antigen. Where the antibody is one which binds CD9, it will usually preferentially bind CD9 as opposed to other receptors, and does not include incidental binding such as non-specific Fc contact, or binding to post-translational modifications common to other antigens and may be one which does not significantly cross-react with other proteins. Methods, for the detection of an antibody that binds an antigen of interest, are well known in the art and can include but are not limited to assays such as FACS, cell ELISA and Western blot.

As used herein, the expressions “cell”, “cell line”, and “cell culture” are used interchangeably, and all such designations include progeny. It is also understood that all progeny may not be precisely identical in DNA content, due to deliberate or inadvertent mutations. Mutant progeny that have the same function or biological activity as screened for in the originally transformed cell are included. It will be clear from the context where distinct designations are intended.

“Treatment or treating” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. Hence, the mammal to be treated herein may have been diagnosed as having the disorder or may be predisposed or susceptible to the disorder.

The terms “cancer” and “cancerous” refer to or describe the physiological condition in mammals that is typically characterized by unregulated cell growth or death. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, camomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Aventis, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

“Mammal” for purposes of treatment refers to any animal classified as a mammal, including humans, mice, SCID or nude mice or strains of mice, domestic and farm animals, and zoo, sports, or pet animals, such as sheep, dogs, horses, cats, cows, etc. Preferably, the mammal herein is human.

“Oligonucleotides” are short-length, single- or double-stranded polydeoxynucleotides that are chemically synthesized by known methods (such as phosphotriester, phosphite, or phosphoramidite chemistry, using solid phase techniques such as described in EP 266,032, published 4 May 1988, or via deoxynucleoside H-phosphonate intermediates as described by Froehler et al., Nucl. Acids Res., 14:5399-5407, 1986. They are then purified on polyacrylamide gels.

In accordance with the present invention, “humanized” and/or “chimeric” forms of non-human (e.g. murine) immunoglobulins refer to antibodies which contain specific chimeric immunoglobulins, immunoglobulin chains or fragments thereof (such as Fv, Fab, Fab′, F(ab′)₂ or other antigen-binding subsequences of antibodies) which results in the decrease of a human anti-mouse antibody (HAMA), human anti-chimeric antibody (HACA) or a human anti-human antibody (HAHA) response, compared to the original antibody, and contain the requisite portions (e.g. CDR(s), antigen binding region(s), variable domain(s) and so on) derived from said non-human immunoglobulin, necessary to reproduce the desired effect, while simultaneously retaining binding characteristics which are comparable to said non-human immunoglobulin. For the most part, humanized antibodies are human immunoglobulins (recipient antibody) in which residues from the complementarity determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues which are found neither in the recipient antibody nor in the imported CDR or FR sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an immunoglobulin constant region (Fc), typically that of a human immunoglobulin.

“De-immunized” antibodies are immunoglobulins that are non-immunogenic, or less immunogenic, to a given species. De-immunization can be achieved through structural alterations to the antibody. Any de-immunization technique known to those skilled in the art can be employed. One suitable technique for de-immunizing antibodies is described, for example, in WO 00/34317 published Jun. 15, 2000.

An antibody which induces “apoptosis” is one which induces programmed cell death by any means, illustrated by but not limited to binding of annexin V, caspase activity, fragmentation of DNA, cell shrinkage, dilation of endoplasmic reticulum, cell fragmentation, and/or formation of membrane vesicles (called apoptotic bodies).

As used herein “antibody induced cytotoxicity” is understood to mean the cytotoxic effect derived from the hybridoma supernatant or antibody produced by the hybridoma deposited with the IDAC as accession number 141204-01, a humanized antibody of the isolated monoclonal antibody produced by the hybridoma deposited with the IDAC as accession number 141204-01, a chimeric antibody of the isolated monoclonal antibody produced by the hybridoma deposited with the IDAC as accession number 141204-01, antigen binding fragments, or antibody ligands thereof, which effect is not necessarily related to the degree of binding.

Throughout the instant specification, hybridoma cell lines, as well as the isolated monoclonal antibodies which are produced therefrom, are alternatively referred to by their internal designation, AR40A746.2.3 or Depository Designation, IDAC 141204-01.

As used herein “antibody-ligand” includes a moiety which exhibits binding specificity for at least one epitope of the target antigen, and which may be an intact antibody molecule, antibody fragments, and any molecule having at least an antigen-binding region or portion thereof (i.e., the variable portion of an antibody molecule), e.g., an Fv molecule, Fab molecule, Fab′ molecule, F(ab′).sub.2 molecule, a bispecific antibody, a fusion protein, or any genetically engineered molecule which specifically recognizes and binds at least one epitope of the antigen bound by the isolated monoclonal antibody produced by the hybridoma cell line designated as IDAC 141204-01 (the IDAC 141204-01 antigen), a humanized antibody of the isolated monoclonal antibody produced by the hybridoma deposited with the IDAC as accession number 141204-01, a chimeric antibody of the isolated monoclonal antibody produced by the hybridoma deposited with the IDAC as accession number 141204-01 and antigen binding fragments.

As used herein “cancerous disease modifying antibodies” (CDMAB) refers to monoclonal antibodies which modify the cancerous disease process in a manner which is beneficial to the patient, for example by reducing tumor burden or prolonging survival of tumor bearing individuals, and antibody-ligands thereof.

A “CDMAB related binding agent”, in its broadest sense, is understood to include, but is not limited to, any form of human or non-human antibodies, antibody fragments, antibody ligands, or the like, which competitively bind to at least one CDMAB target epitope.

A “competitive binder” is understood to include any form of human or non-human antibodies, antibody fragments, antibody ligands, or the like which has binding affinity for at least one CDMAB target epitope.

Tumors to be treated include primary tumors and metastatic tumors, as well as refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents but recur up to five years, sometimes up to ten years or longer after treatment is discontinued.

Tumors that can be treated include tumors that are not vascularized, or not yet substantially vascularized, as well as vascularized tumors. Examples of solid tumors, which can be accordingly treated, include breast carcinoma, lung carcinoma, colorectal carcinoma, pancreatic carcinoma, glioma and lymphoma. Some examples of such tumors include epidernoid tumors, squamous tumors, such as head and neck tumors, colorectal tumors, prostate tumors, breast tumors, lung tumors, including small cell and non-small cell lung tumors, pancreatic tumors, thyroid tumors, ovarian tumors, and liver tumors. Other examples include Kaposi's sarcoma, CNS neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma.

As used herein “antigen-binding region” means a portion of the molecule which recognizes the target antigen.

As used herein “competitively inhibits” means being able to recognize and bind a determinant site to which the monoclonal antibody produced by the hybridoma cell line designated as IDAC 141204-01, (the IDAC 141204-01 antibody), a humanized antibody of the isolated monoclonal antibody produced by the hybridoma deposited with the IDAC as accession number 141204-01, a chimeric antibody of the isolated monoclonal antibody produced by the hybridoma deposited with the IDAC as accession number 141204-01, antigen binding fragments, or antibody ligands thereof, is directed using conventional reciprocal antibody competition assays. (Belanger L., Sylvestre C. and Dufour D. (1973), Enzyme linked immunoassay for alpha fetoprotein by competitive and sandwich procedures. Clinica Chimica Acta 48, 15).

As used herein “target antigen” is the IDAC 141204-01 antigen or portions thereof.

As used herein, an “immunoconjugate” means any molecule or CDMAB such as an antibody chemically or biologically linked to cytotoxins, radioactive agents, cytokines, interferons, target or reporter moieties, enzymes, toxins, anti-tumor drugs or therapeutic agents. The antibody or CDMAB may be linked to the cytotoxin, radioactive agent, cytokine, interferon, target or reporter moiety, enzyme, toxin, anti-tumor drug or therapeutic agent at any location along the molecule so long as it is able to bind its target. Examples of immunoconjugates include antibody toxin chemical conjugates and antibody-toxin fusion proteins.

Radioactive agents suitable for use as anti-tumor agents are known to those skilled in the art. For example, 131I or 211At is used. These isotopes are attached to the antibody using conventional techniques (e.g. Pedley et al., Br. J. Cancer 68, 69-73 (1993)). Alternatively, the anti-tumor agent which is attached to the antibody is an enzyme which activates a prodrug. A prodrug may be administered which will remain in its inactive form until it reaches the tumor site where it is converted to its cytotoxin form once the antibody complex is administered. In practice, the antibody-enzyme conjugate is administered to the patient and allowed to localize in the region of the tissue to be treated. The prodrug is then administered to the patient so that conversion to the cytotoxic drug occurs in the region of the tissue to be treated. Alternatively, the anti-tumor agent conjugated to the antibody is a cytokine such as interleukin-2 (IL-2), interleukin-4 (IL-4) or tumor necrosis factor alpha (TNF-α). The antibody targets the cytokine to the tumor so that the cytokine mediates damage to or destruction of the tumor without affecting other tissues. The cytokine is fused to the antibody at the DNA level using conventional recombinant DNA techniques. Interferons may also be used.

As used herein, a “fusion protein” means any chimeric protein wherein an antigen binding region is connected to a biologically active molecule, e.g., toxin, enzyme, fluorescent proteins, luminescent marker, polypeptide tag, cytokine, interferon, target or reporter moiety or protein drug.

The invention further contemplates CDMAB of the present invention to which target or reporter moieties are linked. Target moieties are first members of binding pairs. Anti-McHale tumor agents, for example, are conjugated to second members of such pairs and are thereby directed to the site where the antigen-binding protein is bound. A common example of such a binding pair is avidin and biotin. In a preferred embodiment, biotin is conjugated to the target antigen of the CDMAB of the present invention, and thereby provides a target for an anti-tumor agent or other moiety which is conjugated to avidin or streptavidin. Alternatively, biotin or another such moiety is linked to the target antigen of the CDMAB of the present invention and used as a reporter, for example in a diagnostic system where a detectable signal-producing agent is conjugated to avidin or streptavidin.

Detectable signal-producing agents are useful in vivo and in vitro for diagnostic purposes. The signal producing agent produces a measurable signal which is detectable by external means, usually the measurement of electromagnetic radiation. For the most part, the signal producing agent is an enzyme or chromophore, or emits light by fluorescence, phosphorescence or chemiluminescence. Chromophores include dyes which absorb light in the ultraviolet or visible region, and can be substrates or degradation products of enzyme catalyzed reactions.

Moreover, included within the scope of the present invention is use of the present CDMAB in vivo and in vitro for investigative or diagnostic methods, which are well known in the art. In order to carry out the diagnostic methods as contemplated herein, the instant invention may further include kits, which contain CDMAB of the present invention. Such kits will be useful for identification of individuals at risk for certain type of cancers by detecting over-expression of the CDMAB's target antigen on cells of such individuals.

### Diagnostic Assay Kits

It is contemplated to utilize the CDMAB of the present invention in the form of a diagnostic assay kit for determining the presence of a tumor. The tumor will generally be detected in a patient based on the presence of one or more tumor-specific antigens, e.g. proteins and/or polynucleotides which encode such proteins in a biological sample, such as blood, sera, urine and/or tumor biopsies, which samples will have been obtained from the patient.

The proteins function as markers which indicate the presence or absence of a particular tumor, for example a colon, breast, lung or prostate tumor. It is further contemplated that the antigen will have utility for the detection of other cancerous tumors. Inclusion in the diagnostic assay kits of binding agents comprised of CDMABs of the present invention, or CDMAB related binding agents, enables detection of the level of antigen that binds to the agent in the biological sample. Polynucleotide primers and probes may be used to detect the level of mRNA encoding a tumor protein, which is also indicative of the presence or absence of a cancer. In order for the binding assay to be diagnostic, data will have been generated which correlates statistically significant levels of antigen, in relation to that present in normal tissue, so as to render the recognition of binding definitively diagnostic for the presence of a cancerous tumor. It is contemplated that a plurality of formats will be useful for the diagnostic assay of the present invention, as are known to those of ordinary skill in the art, for using a binding agent to detect polypeptide markers in a sample. For example, as illustrated in Harlow and Lane, Antibodies: A Laboratory Manual, Cold Spring Harbor Laboratory, 1988. Further contemplated are any and all combinations, permutations or modifications of the afore-described diagnostic assay formats.

The presence or absence of a cancer in a patient will typically be determined by (a) contacting a biological sample obtained from a patient with a binding agent; (b) detecting in the sample a level of polypeptide that binds to the binding agent; and (c) comparing the level of polypeptide with a predetermined cut-off value.

In an illustrative embodiment, it is contemplated that the assay will involve the use of a CDMAB based binding agent immobilized on a solid support to bind to and remove the polypeptide from the remainder of the sample. The bound polypeptide may then be detected using a detection reagent that contains a reporter group and specifically binds to the binding agent/polypeptide complex. Illustrative detection reagents may include a CDMAB based binding agent that specifically binds to the polypeptide or an antibody or other agent that specifically binds to the binding agent, such as an anti-immunoglobulin, protein G, protein A or a lectin. In an alternative embodiment, it is contemplated that a competitive assay may be utilized, in which a polypeptide is labeled with a reporter group and allowed to bind to the immobilized binding agent after incubation of the binding agent with the sample. Indicative of the reactivity of the sample with the immobilized binding agent, is the extent to which components of the sample inhibit the binding of the labeled polypeptide to the binding agent. Suitable polypeptides for use within such assays include full length tumor-specific proteins and/or portions thereof, to which the binding agent has binding affinity.

The diagnostic kit will be provided with a solid support which may be in the form of any material known to those of ordinary skill in the art to which the protein may be attached. Suitable examples may include a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

It is contemplated that the binding agent will be immobilized on the solid support using a variety of techniques known to those of skill in the art, which are amply described in the patent and scientific literature. The term “immobilization” refers to both noncovalent association, such as adsorption, and covalent attachment, which, in the context of the present invention, may be a direct linkage between the agent and functional groups on the support, or may be a linkage by way of a cross-linking agent. In a preferred, albeit non-limiting embodiment, immobilization by adsorption to a well in a microtiter plate or to a membrane is preferable. Adsorption may be achieved by contacting the binding agent, in a suitable buffer, with the solid support for a suitable amount of time. The contact time may vary with temperature, and will generally be within a range of between about 1 hour and about 1 day.

Covalent attachment of binding agent to a solid support would ordinarily be accomplished by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12 A13).

It is further contemplated that the diagnostic assay kit will take the form of a two-antibody sandwich assay. This assay may be performed by first contacting an antibody, e.g. the instantly disclosed CDMAB that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

In a specific embodiment, it is contemplated that once the antibody is immobilized on the support as described above, the remaining protein binding sites on the support will be blocked, via the use of any suitable blocking agent known to those of ordinary skill in the art, such as bovine serum albumin or Tween 20™ (Sigma Chemical Co., St. Louis, Mo.). The immobilized antibody would then be incubated with the sample, and polypeptide would be allowed to bind to the antibody. The sample could be diluted with a suitable diluent, such as phosphate-buffered saline (PBS) prior to incubation. In general, an appropriate contact time (i.e., incubation time) would be selected to correspond to a period of time sufficient to detect the presence of polypeptide within a sample obtained from an individual with the specifically selected tumor. Preferably, the contact time is sufficient to achieve a level of binding that is at least about 95 percent of that achieved at equilibrium between bound and unbound polypeptide. Those of ordinary skill in the art will recognize that the time necessary to achieve equilibrium may be readily determined by assaying the level of binding that occurs over a period of time.

It is further contemplated that unbound sample would then be removed by washing the solid support with an appropriate buffer. The second antibody, which contains a reporter group, would then be added to the solid support. Incubation of the detection reagent with the immobilized antibody-polypeptide complex would then be carried out for an amount of time sufficient to detect the bound polypeptide. Subsequently, unbound detection reagent would then be removed and bound detection reagent would be detected using the reporter group. The method employed for detecting the reporter group is necessarily specific to the type of reporter group selected, for example for radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

In order to utilize the diagnostic assay kit of the present invention to determine the presence or absence of a cancer, such as prostate cancer, the signal detected from the reporter group that remains bound to the solid support would generally be compared to a signal that corresponds to a predetermined cut-off value. For example, an illustrative cut-off value for the detection of a cancer may be the average mean signal obtained when the immobilized antibody is incubated with samples from patients without the cancer. In general, a sample generating a signal that is about three standard deviations above the predetermined cut-off value would be considered positive for the cancer. In an alternate embodiment, the cut-off value might be determined by using a Receiver Operator Curve, according to the method of Sackett et al., Clinical Epidemiology. A Basic Science for Clinical Medicine, Little Brown and Co., 1985, p. 106-7. In such an embodiment, the cut-off value could be determined from a plot of pairs of true positive rates (i.e., sensitivity) and false positive rates (100 percent-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

It is contemplated that the diagnostic assay enabled by the kit will be performed in either a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound will be immersed in a solution containing the sample. The sample migrates along the membrane through a region containing second binding agent and to the area of immobilized binding agent. Concentration of the second binding agent at the area of immobilized antibody indicates the presence of a cancer. Generation of a pattern, such as a line, at the binding site, which can be read visually, will be indicative of a positive test. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in the instant diagnostic assay are the instantly disclosed antibodies, antigen-binding fragments thereof, and any CDMAB related binding agents as herein described. The amount of antibody immobilized on the membrane will be any amount effective to produce a diagnostic assay, and may range from about 25 nanograms to about 1 microgram. Typically such tests may be performed with a very small amount of biological sample.

Additionally, the CDMAB of the present invention may be used in the laboratory for research due to its ability to identify its target antigen.

In order that the invention herein described may be more fully understood, the following description is set forth.

The present invention provides CDMAB (i.e., IDAC 141204-01 CDMAB, a humanized antibody of the isolated monoclonal antibody produced by the hybridoma deposited with the IDAC as accession number 141204-01, a chimeric antibody of the isolated monoclonal antibody produced by the hybridoma deposited with the IDAC as accession number 141204-01, antigen binding fragments, or antibody ligands thereof) which specifically recognize and bind the IDAC 141204-01 antigen.

The CDMAB of the isolated monoclonal antibody produced by the hybridoma deposited with the IDAC as accession number 141204-01 may be in any form as long as it has an antigen-binding region which competitively inhibits the immunospecific binding of the isolated monoclonal antibody produced by hybridoma IDAC 141204-01 to its target antigen. Thus, any recombinant proteins (e.g., fusion proteins wherein the antibody is combined with a second protein such as a lymphokine or a tumor inhibitory growth factor) having the same binding specificity as the IDAC 141204-01 antibody fall within the scope of this invention.

In one embodiment of the invention, the CDMAB is the IDAC 141204-01 antibody.

In other embodiments, the CDMAB is an antigen binding fragment which may be a Fv molecule (such as a single-chain Fv molecule), a Fab molecule, a Fab′ molecule, a F(ab′)2 molecule, a fusion protein, a bispecific antibody, a heteroantibody or any recombinant molecule having the antigen-binding region of the IDAC 141204-01 antibody. The CDMAB of the invention is directed to the epitope to which the IDAC 141204-01 monoclonal antibody is directed.

The CDMAB of the invention may be modified, i.e., by amino acid modifications within the molecule, so as to produce derivative molecules. Chemical modification may also be possible. Modification by direct mutation, methods of affinity maturation, phage display or chain shuffling may also be possible.

Affinity and specificity can be modified or improved by mutating CDR and/or phenylalanine tryptophan (FW) residues and screening for antigen binding sites having the desired characteristics (e.g., Yang et al., J. Mol. Biol., (1995) 254: 392-403). One way is to randomize individual residues or combinations of residues so that in a population of otherwise identical antigen binding sites, subsets of from two to twenty amino acids are found at particular positions. Alternatively, mutations can be induced over a range of residues by error prone PCR methods (e.g., Hawkins et al., J. Mol. Biol., (1992) 226: 889-96). In another example, phage display vectors containing heavy and light chain variable region genes can be propagated in mutator strains of E. coli (e.g., Low et al., J. Mol. Biol., (1996) 250: 359-68). These methods of mutagenesis are illustrative of the many methods known to one of skill in the art.

Another manner for increasing affinity of the antibodies of the present invention is to carry out chain shuffling, where the heavy or light chain are randomly paired with other heavy or light chains to prepare an antibody with higher affinity. The various CDRs of the antibodies may also be shuffled with the corresponding CDRs in other antibodies. Derivative molecules would retain the functional property of the polypeptide, namely, the molecule having such substitutions will still permit the binding of the polypeptide to the IDAC 141204-01 antigen or portions thereof.

These amino acid substitutions include, but are not necessarily limited to, amino acid substitutions known in the art as “conservative”.

For example, it is a well-established principle of protein chemistry that certain amino acid substitutions, entitled “conservative amino acid substitutions,” can frequently be made in a protein without altering either the conformation or the function of the protein.

Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments.

### EXAMPLE 1

### Hybridoma Production

### Hybridoma Cell Line AR40A746.2.3

The hybridoma cell line AR40A746.2.3 was deposited, in accordance with the Budapest Treaty, with the International Depository Authority of Canada (IDAC), Bureau of Microbiology, Health Canada, 1015 Arlington Street, Winnipeg, Manitoba, Canada, R3E, 3R2, on Dec. 14, 2004, under Accession Number 141204-01. In accordance with 37 CFR 1.808, the depositors assure that all restrictions imposed on the availability to the public of the deposited materials will be irrevocably removed upon the granting of a patent. The deposit will be replaced if the depository cannot dispense viable samples.

To produce the hybridoma that produces the anti-cancer antibody AR40A746.2.3, a single cell suspension of frozen prostate adenocarcinoma tumor tissue (Genomics Collaborative, Cambridge, Mass.) was prepared in PBS. IMMUNEASY™ (Qiagen, Venlo, Netherlands) adjuvant was prepared for use by gentle mixing. Five to seven week old BALB/c mice were immunized by injecting subcutaneously 2 million cells in 50 microliters of the antigen-adjuvant. Recently prepared antigen-adjuvant was used to boost the immunized mice intraperitoneally, 2 and 3 weeks after the initial immunization, with 2 million cells in 50 microliters. A spleen was used for fusion three days after the last immunization. The hybridomas were prepared by fusing the isolated splenocytes with NSO-1 myeloma partners. The supernatants from the fusions were tested from subclones of the hybridomas.

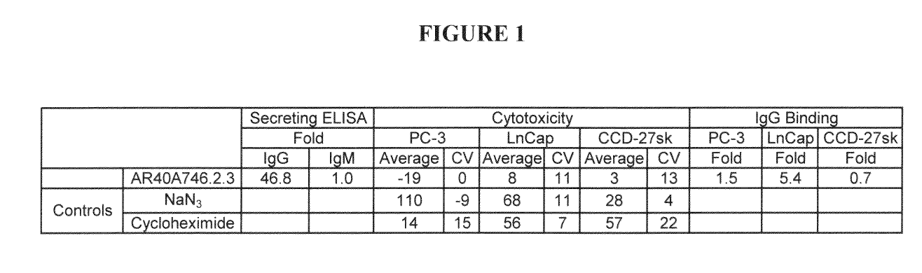

To determine whether the antibodies secreted by the hybridoma cells are of the IgG or IgM isotype, an ELISA assay was employed. 100 microliters/well of goat anti-mouse IgG+IgM (H+ L) at a concentration of 2.4 micrograms/mL in coating buffer (0.1 M carbonate/bicarbonate buffer, pH 9.2-9.6) at 4° C. was added to the ELISA plates overnight. The plates were washed thrice in washing buffer (PBS+0.05 percent Tween). 100 microliters/well blocking buffer (5 percent milk in wash buffer) was added to the plate for 1 hour at room temperature and then washed thrice in washing buffer. 100 microliters/well of hybridoma supernatant was added and the plate incubated for 1 hour at room temperature. The plates were washed thrice with washing buffer and 1/100,000 dilution of either goat anti-mouse IgG or IgM horseradish peroxidase conjugate (diluted in PBS containing 1 percent milk), 100 microliters/well, was added. After incubating the plate for 1 hour at room temperature the plate was washed thrice with washing buffer. 100 microliters/well of TMB solution was incubated for 1-3 minutes at room temperature. The color reaction was terminated by adding 50 microliters/well 2M H₂S0₄ and the plate was read at 450 nm with a Perkin-Elmer HTS7000 plate reader. As indicated in FIG. 1, the AR40A746.2.3 hybridoma secreted primarily antibodies of the IgG isotype.

To determine the subclass of antibody secreted by the hybridoma cells, an isotyping experiment was performed using a Mouse Monoclonal Antibody Isotyping Kit (HyCult Biotechnology, Frontstraat, Netherlands). 500 microliters of buffer solution was added to the test strip containing rat anti-mouse subclass specific antibodies. 500 microliters of hybridoma supernatant was added to the test tube, and submerged by gentle agitation. Captured mouse immunoglobulins were detected directly by a second rat monoclonal antibody which is coupled to colloid particles. The combination of these two proteins creates a visual signal used to analyze the isotype. The anti-cancer antibody AR40A746.2.3 is of the IgG1, kappa isotype.

After one round of limiting dilution, hybridoma supernatants were tested for antibodies that bound to target cells in a cell ELISA assay. Two human prostate cancer cell lines and 1 human non-cancer skin cell line were tested: PC-3, LnCap and CCD-27sk respectively. All cell lines were obtained from the American Type Tissue Collection (ATCC, Manassas, Va.). The plated cells were fixed prior to use. The plates were washed thrice with PBS containing MgCl₂ and CaCl₂ at room temperature. 100 microliters of 2 percent paraformaldehyde diluted in PBS was added to each well for 10 minutes at room temperature and then discarded. The plates were again washed with PBS containing MgCl₂ and CaCl₂ three times at room temperature. Blocking was done with 100 microliters/well of 5 percent milk in wash buffer (PBS+0.05 percent Tween) for 1 hour at room temperature. The plates were washed thrice with wash buffer and the hybridoma supernatant was added at 100 microliters/well for 1 hour at room temperature. The plates were washed 3 times with wash buffer and 100 microliters/well of 1/25,000 dilution of goat anti-mouse IgG antibody conjugated to horseradish peroxidase (diluted in PBS containing 1 percent milk) was added. After 1 hour incubation at room temperature the plates were washed 3 times with wash buffer and 100 microliter/well of TMB substrate was incubated for 1-3 minutes at room temperature. The reaction was terminated with 50 microliters/well 2M H₂S0₄ and the plate read at 450 nm with a Perkin-Elmer HTS7000 plate reader. The results as tabulated in FIG. 1 were expressed as the number of folds above background compared to an in-house IgG isotype control that has previously been shown not to bind to the cell lines tested. The antibodies from the hybridoma AR40A746.2.3 showed binding to the PC-3 and LnCap prostate cancer cell lines with no detectable binding to the non-cancer skin cell line CCD-27sk.

In conjunction with testing for antibody binding, the cytotoxic effect of the hybridoma supernatants (antibody induced cytotoxicity) was tested in the cell lines: PC-3, LnCap and CCD-27sk. Calcein AM was obtained from Molecular Probes (Eugene, Oreg.) and the assay was performed as outlined below. Cells were plated before the assay at the predetermined appropriate density. After 2 days, 100 microliters of supernatant from the hybridoma microtitre plates were transferred to the cell plates and incubated in a 5 percent CO₂ incubator for 5 days. The wells that served as the positive controls were aspirated until empty and 100 microliters of sodium azide (NaN₃, 0.01 percent, Sigma, Oakville, ON) or cycloheximide (CHX, 0.5 micromolar, Sigma, Oakville, ON) dissolved in culture medium, was added. After 5 days of treatment, the plates were then emptied by inverting and blotting dry. Room temperature DPBS (Dulbecco's phosphate buffered saline) containing MgCl₂ and CaCl₂ was dispensed into each well from a multichannel squeeze bottle, tapped 3 times, emptied by inversion and then blotted dry. 50 microliters of the fluorescent calcein dye diluted in DPBS containing MgCl₂ and CaCl₂ was added to each well and incubated at 37° C. in a 5 percent CO₂ incubator for 30 minutes. The plates were read in a Perkin-Elmer HTS7000 fluorescence plate reader and the data was analyzed in Microsoft Excel. The results are tabulated in FIG. 1. Supernatant from the AR40A746.2.3 hybridoma produced specific cytotoxicity of 8 percent on the LnCap prostate cancer cells. This was 12 and 14 percent of the cytotoxicity obtained with the positive controls sodium azide and cycloheximide on the LnCap prostate cancer cells, respectively.

Results from FIG. 1 demonstrate that the cytotoxic effects of AR40A746.2.3 correlate with the binding levels on the cancer cell types. The strongest detectable binding was to the LnCap prostate cancer cells and similarly the highest detectable cytotoxicity was also on the LnCap prostate cancer cells. As tabulated in FIG. 1, AR40A746.2.3 did not produce cytotoxicity in the CCD-27sk non-cancer human skin cell line. The known non-specific cytotoxic agents cycloheximide and NaN₃ generally produced cytotoxicity as expected.

### EXAMPLE 2

### In vitro Binding

AR40A746.2.3 monoclonal antibody was produced by culturing the hybridoma in CL-1000 flasks (BD Biosciences, Oakville, ON) with collections and reseeding occurring twice/week. Standard antibody purification procedures with Protein G Sepharose 4 Fast Flow (Amersham Biosciences, Baie d'Urfé, Q C) were followed. It is within the scope of this invention to utilize monoclonal antibodies that are de-immunized, humanized, chimeric or murine.

Binding of AR40A746.2.3 to colon (DLD-1, HT-29, Lovo and SW1116), pancreatic (BxPC-3), breast (MDA-MB-231 and MCF-7), prostate (PC-3 and DU-145), ovarian (OVCAR-3) and melanoma (A2058, A375, WM9, WM35, WM164, WM451, WM537, WM852, WM983, WM1205 and WM1232) cancer, and non-cancer cell lines from skin (CCD-27sk) and lung (Hs888.Lu) was assessed by flow cytometry (FACS). All cell lines were obtained from the American Type Tissue Collection (ATCC, Manassas, Va.) except for the melanoma cell lines WM9, WM35, WM164, WM451, WM537, WM852, WM983, WM1205 and WM1232 which were obtained from Dr. David Hogg (University of Toronto, Toronto, Canada).

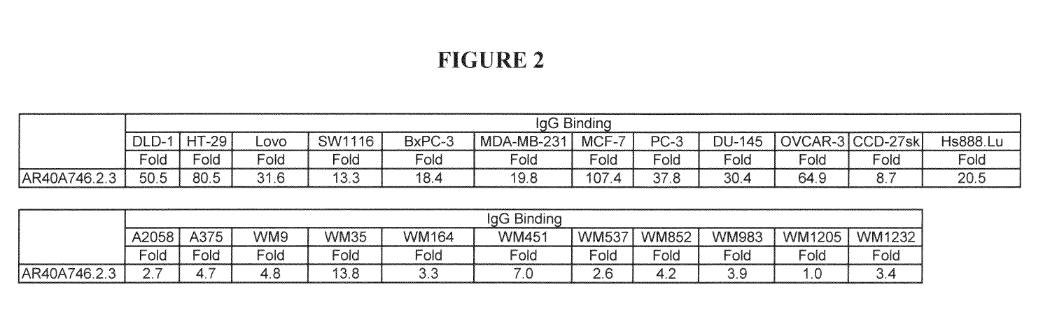

Cells were prepared for FACS by initially washing the cell monolayer with DPBS (without Ca⁺⁺ and Mg⁺⁺). Cell dissociation buffer (Invitrogen, Burlington, ON) was then used to dislodge the cells from their cell culture plates at 37° C. After centrifugation and collection, the cells were resuspended in DPBS containing MgCl₂ CaCl₂ and 2 percent fetal bovine serum at 4° C. (staining media) and counted, aliquoted to appropriate cell density, spun down to pellet the cells and resuspended in staining media at 4° C. in the presence of the test antibody (AR40A746.2.3) or control antibodies (isotype control, anti-EGFR). Isotype control and the test antibody were assessed at 20 micrograms/mL whereas anti-EGFR was assessed at 5 micrograms/mL on ice for 30 minutes. Prior to the addition of Alexa Fluor 546-conjugated secondary antibody the cells were washed once with staining media. The Alexa Fluor 546-conjugated antibody in staining media was then added for 30 minutes at 4° C. The cells were then washed for the final time and resuspended in fixing media (staining media containing 1.5 percent paraformaldehyde). Flow cytometric acquisition of the cells was assessed by running samples on a FACSarray™ using the FACSarray™ System Software (BD Biosciences, Oakville, ON). The forward (FSC) and side scatter (SSC) of the cells were set by adjusting the voltage and amplitude gains on the FSC and SSC detectors. The detectors for the fluorescence (Alexa-546) channel was adjusted by running unstained cells such that cells had a uniform peak with a median fluorescent intensity of approximately 1-5 units. For each sample, approximately 10,000 gated events (stained fixed cells) were acquired for analysis and the results are presented in FIG. 2.

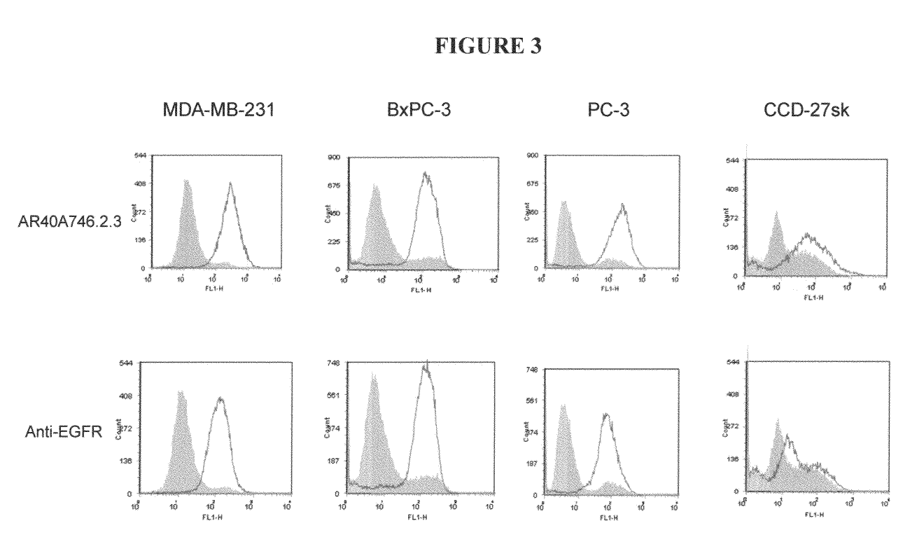

FIG. 2 presents the mean fluorescence intensity fold increase above isotype control. Representative histograms of AR40A746.2.3 antibodies were compiled for FIG. 3. AR40A746.2.3 demonstrated strong binding to the colon DLD-1 (50.5-fold), HT-29 (80.5-fold) and Lovo (31.6-fold), breast MCF-7 (107.4-fold), prostate PC-3 (37.8-fold) and DU-145 (30.4-fold) and ovarian OVCAR-3 (64.9-fold) human cancer cell lines. There was also binding to colon SW1116 (13.3-fold), pancreatic BxPC-3 (18.4-fold), breast MDA-MB-231 (19.8-fold) and melanoma A2058 (2.7-fold), A375 (4.7-fold), WM9 (4.8-fold), WM35 (13.8-fold), WM164 (3.3-fold), WM451 (7.0-fold), WM537 (2.6-fold), WM852 (4.2-fold), WM983 (3.9-fold) and WM1232 (3.4-fold) human cancer cell lines. There was detectable binding to the human non-cancer skin CCD-27sk (8.7-fold) and lung Hs888.Lu (20.5-fold). There was no detectable binding to the melanoma cancer cell line WM1205. These data demonstrate that AR40A746.2.3 bound to several different cancer cell lines with varying levels of antigen expression.

### EXAMPLE 3

### In vivo Tumor Experiment with human BxPC-3 Pancreatic Cancer Cells

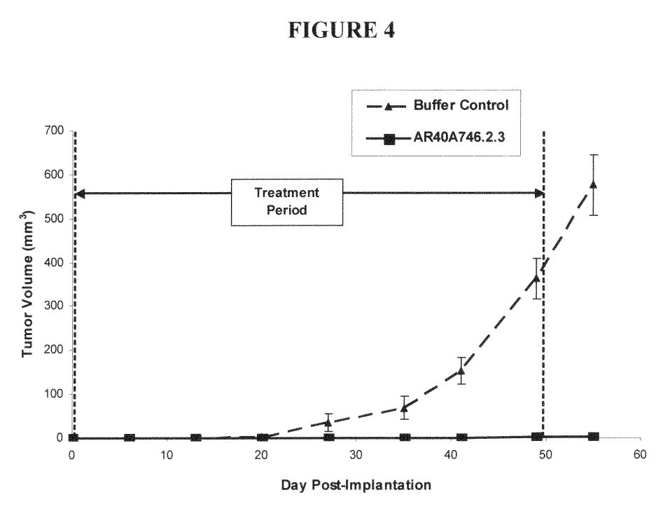

In Example 1, AR40A746.2.3 demonstrated cytotoxicity against human cancer cells in vitro. To extend this finding to an in vivo model, AR40A746.2.3 was tested in a human BxPC-3 pancreatic cancer xenograft model. With reference to FIGS. 4 and 5, 8 to 10 week old female SCID mice were implanted with 5 million human pancreatic cancer cells (BxPC-3) in 100 microliters PBS solution injected subcutaneously in the scruff of the neck. The mice were randomly divided into 2 treatment groups of 5. On the day after implantation, 20 mg/kg of AR40A746.2.3 test antibody or buffer control was administered intraperitoneally to each cohort in a volume of 300 microliters after dilution from the stock concentration with a diluent that contained 2.7 mM KCl, 1 mM KH₂PO₄, 137 mM NaCl and 20 mM Na₂HPO₄. The antibody and control samples were then administered once per week for the duration of the study. Tumor growth was measured about every 7 days with calipers. The study was completed after 8 doses of antibody. Body weights of the animals were recorded once per week for the duration of the study. At the end of the study all animals were euthanized according to CCAC guidelines.

AR40A746.2.3 reduced tumor growth in the BxPC-3 in vivo prophylactic model of human pancreatic cancer. Treatment with Arius antibody AR40A746.2.3 significantly reduced the growth of BxPC-3 tumors by 99.56 percent (p<0.0001, t-test), compared to the buffer-treated group, as determined on day 55, 5 days after the last dose of antibody (FIG. 4).

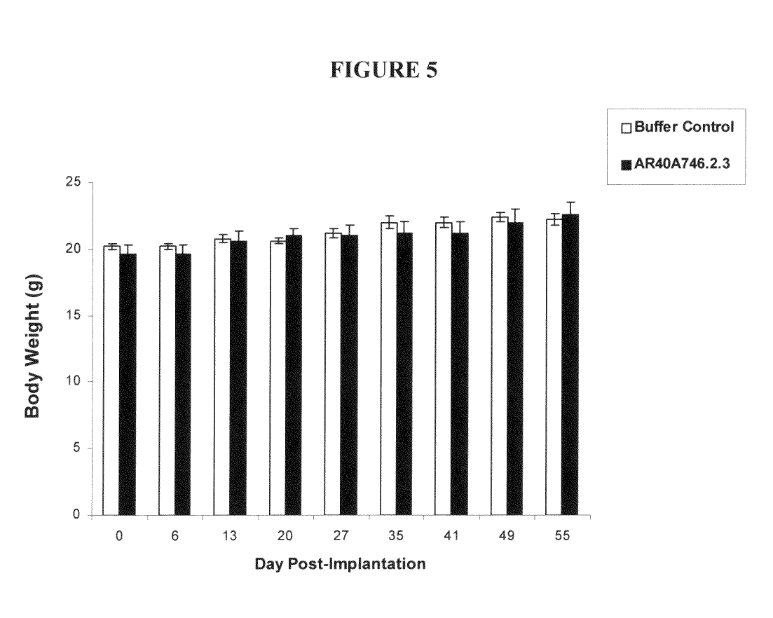

There were no clinical signs of toxicity throughout the study. Body weight measured at weekly intervals was a surrogate for well-being and failure to thrive. The mean body weight increased in all groups over the duration of the study (FIG. 5). The mean weight gain between day 0 and day 55 was 2.0 g (9.9 percent) in the control group and 3.0 g (15.3 percent) in the AR40A746.2.3-treated group. There were no significant differences between the groups at the end of the treatment period.

In summary, AR40A746.2.3 was well-tolerated and decreased the tumor burden in this human pancreatic cancer xenograft model.

### EXAMPLE 4

### In Vivo Tumor Experiment with Human BxPC-3 Pancreatic Cancer Cells

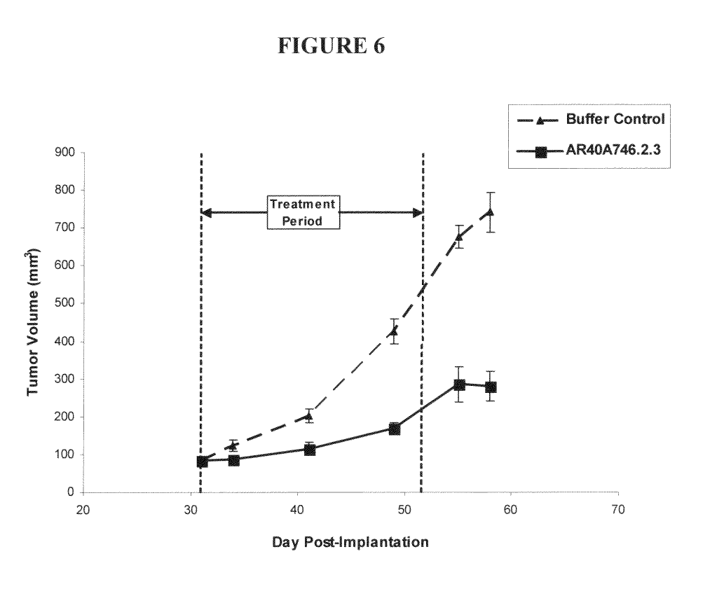

In Example 3, AR40A746.2.3 demonstrated efficacy against a human prophylactic pancreatic xenograft cancer model. To extend this finding to an established model, AR40A746.2.3 was tested in an established human BxPC-3 pancreatic cancer xenograft model. With reference to FIGS. 6 and 7, 8 to 10 week old female SCID mice were implanted with 5 million human pancreatic cancer cells (BxPC-3) in 100 microliters PBS solution injected subcutaneously in the neck scruff of each mouse. The mice were randomly divided into 2 treatment groups of 8 when the average mouse tumor volume reached approximately 83 mm³. On day 31 after implantation, 20 mg/kg of AR40A746.2.3 test antibody or buffer control was administered intraperitoneally to each cohort in a volume of 300 microliters after dilution from the stock concentration with a diluent that contained 2.7 mM KCl, 1 mM KH₂PO₄, 137 mM NaCl and 20 mM Na₂HPO₄. The antibody and control samples were then administered three times per week for around 3 weeks. Tumor growth was measured once per week with calipers. The treatment was completed after 10 doses of antibody. Body weights of the animals were recorded at the same time as tumor measurement. All animals were euthanized according to CCAC guidelines at the end of the study once they had reached endpoint.

AR40A746.2.3 demonstrated significant inhibition of tumor growth in the BxPC-3 in vivo established model of human pancreatic cancer. Treatment with Arius antibody AR40A746.2.3 reduced the growth of BxPC-3 tumors by 70.14 percent (p=0.00001, t-test), compared to the buffer-treated group, as determined on day 58, 6 days after last dose of antibody (FIG. 6).

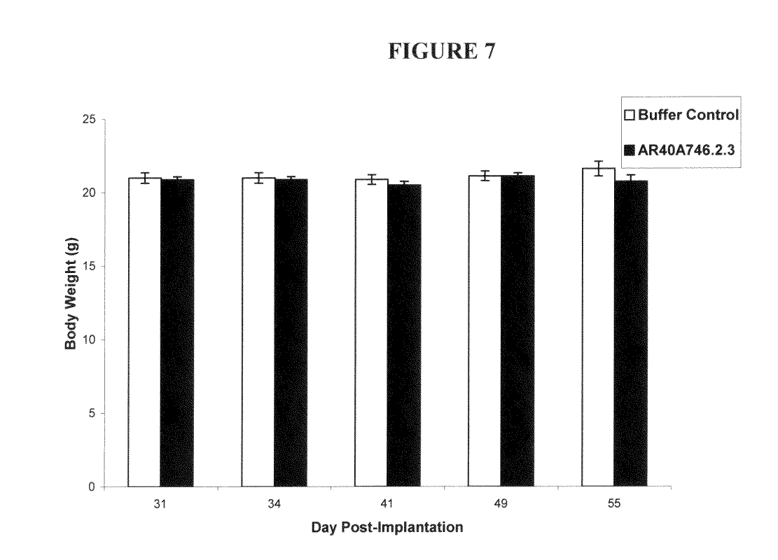

There were no obvious clinical signs of toxicity throughout the study. Body weight measured at weekly intervals was a surrogate for well being and failure to thrive. The mean body weight remained about the same in all groups over the duration of the study (FIG. 7). There were no significant differences between the groups during the treatment period.

In summary, AR40A746.2.3 was well-tolerated and significantly inhibited the tumor growth in this established human pancreatic cancer xenograft model.

### EXAMPLE 5

### In Vivo Tumor Experiment with Human MDA-MB-231 Breast Cancer Cells

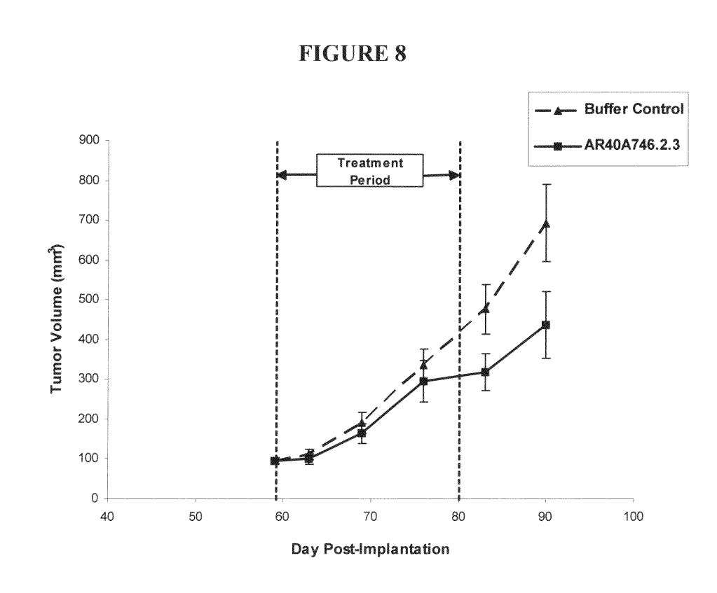

In Examples 3 and 4, AR40A746.2.3 demonstrated efficacy against human pancreatic xenograft cancer models. To extend this finding to a breast cancer model, AR40A746.2.3 was tested in an established human MDA-MB-231 breast cancer xenograft model. With reference to FIGS. 8 and 9, 8 to 10 week old female SCID mice were implanted with 5 million human breast cancer cells (MDA-MB-231) in 100 microliters PBS solution injected subcutaneously in the neck scruff of each mouse. The mice were randomly divided into 2 treatment groups of 10 when the average mouse tumor volume reached approximately 100 mm³. On day 59 after implantation, 20 mg/kg of AR40A746.2.3 test antibody or buffer control was administered intraperitoneally to each cohort in a volume of 300 microliters after dilution from the stock concentration with a diluent that contained 2.7 mM KCl, 1 mM KH₂PO₄, 137 mM NaCl and 20 mM Na₂HPO₄. The antibody and control samples were then administered three times per week for around 3 weeks. Tumor growth was measured once per week with calipers. The treatment was completed after 10 doses of antibody. Body weights of the animals were recorded at the same time as tumor measurement. All animals were euthanized according to Canadian Council on Animal Care (CCAC) guidelines at the end of the study once they had reached endpoint.

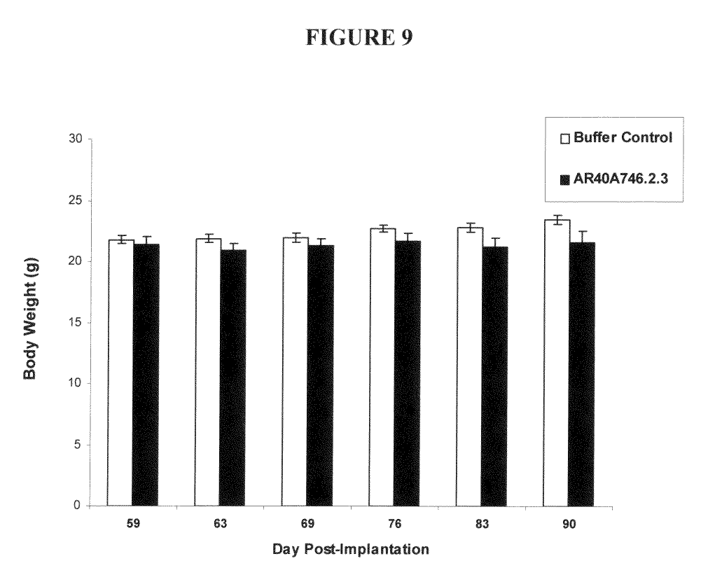

AR40A746.2.3 demonstrated inhibition of tumor growth in the MDA-MB-231 in vivo established model of human breast cancer. Treatment with Arius antibody AR40A746.2.3 reduced the growth of MDA-MB-231 tumors by 42.67 percent (p=0.08, t-test), compared to the buffer-treated group, as determined on day 90, 10 days after the last dose of antibody (FIG. 8). There were no obvious clinical signs of toxicity throughout the study. Body weight measured at weekly intervals was a surrogate for well being and failure to thrive. The mean body weight increased in all groups over the duration of the study (FIG. 9). The mean weight gain between day 59 and day 90 was 1.64 g (7.0 percent) in the control group and 0.17 g (0.8 percent) in the AR40A736.2.3-treated group. There were no significant differences between the groups during the treatment period.

In summary, AR40A746.2.3 was well-tolerated and inhibited the tumor growth in this human breast cancer xenograft model. AR40A746.2.3 has demonstrated efficacy against three different human cancer indications: prostate, pancreatic and breast. Treatment benefits were observed in several well-recognized models of human cancer disease suggesting pharmacologic and pharmaceutical benefits of this antibody for therapy in other mammals, including man. In toto, this data demonstrates that the AR40A746.2.3 antigen is a cancer associated antigen and is expressed on human cancer cells, and is a pathologically relevant cancer target.

### EXAMPLE 6

### In Vivo Tumor Experiment with human BxPC-3 Pancreatic Cancer Cells

In Examples 3 and 4, AR40A746.2.3 demonstrated efficacy in both a prophylactic and an established BxPC-3 human pancreatic cancer xenograft model. To determine effective dose levels, AR40A746.2.3 was tested in an established BxPC-3 model at various doses. With reference to FIGS. 10 and 11, 8 to 10 week old female SCID mice were implanted with 5 million human pancreatic cancer cells (BxPC-3) in 100 microliters PBS solution injected subcutaneously in the neck scruff of each mouse. The mice were randomly divided into 5 treatment groups of 9 when the average mouse tumor volume reached approximately 83 mm³. On day 30 after implantation, 20, 10, 5 or 2 mg/kg of AR40A746.2.3 test antibody or buffer control was administered intraperitoneally to each cohort in a volume of 300 microliters after dilution from the stock concentration with a diluent that contained 2.7 mM KCl, 1 mM KH₂PO₄, 137 mM NaCl and 20 mM Na₂HPO₄. The antibody and control samples were then administered three times per week for around 3 weeks. Tumor growth was measured once every week with calipers. The treatment was completed after 10 doses of antibody. Body weights of the animals were recorded at the same time as tumor measurement. All animals were euthanized according to CCAC guidelines at the end of the study once they had reached endpoint.

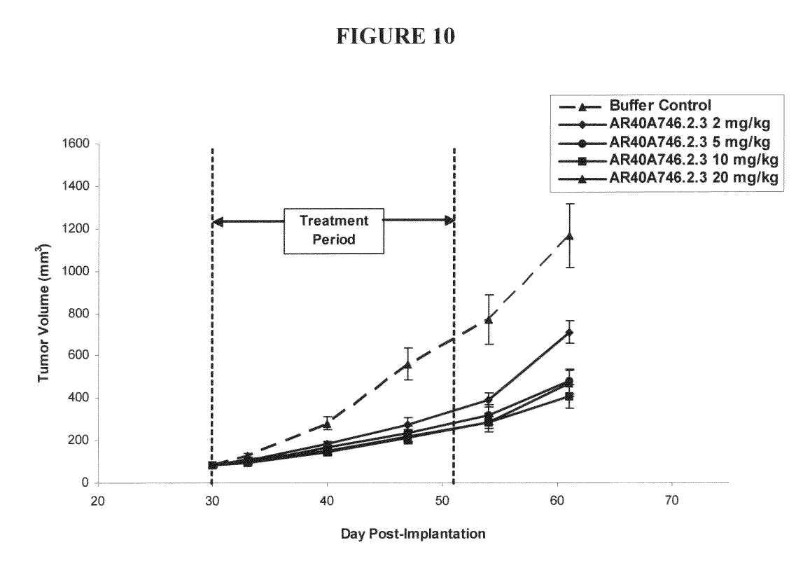

AR40A746.2.3 demonstrated dose-dependent inhibition of tumor growth in the in vivo established model of human pancreatic cancer. Treatment with Arius antibody AR40A746.2.3 at doses of 20, 10, 5 or 2 mg/kg reduced the growth of BxPC-3 tumors by 64.7 percent (p<0.0003, t-test), 69.9 percent (p<0.0001, t-test), 63.7 percent (p<0.0003, t-test) or 42.0 percent (p<0.0074, t-test), compared to the buffer-treated group, as determined on day 61, 10 days after last dose of antibody (FIG. 10). Maximum inhibition was obtained at the 20, 10 and 5 mg/kg doses.

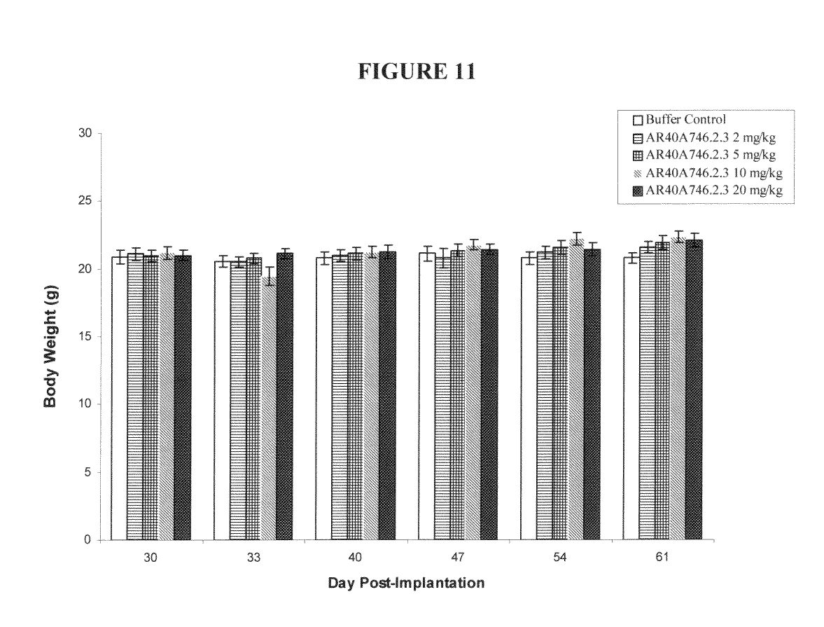

There were no obvious clinical signs of toxicity throughout the study. Body weight measured at weekly intervals was a surrogate for well being and failure to thrive. The mean body weight remained about the same in all the groups over the duration of the study (FIG. 11). There were no significant differences between the groups during the treatment period.

In summary, AR40A746.2.3 was well-tolerated and significantly inhibited, at all tested doses, the tumor growth in a dose dependent manner in this established human pancreatic cancer xenograft model. In toto, this data demonstrates that AR40A746.2.3 is effective in the treatment of human cancer in a dose dependent manner.

### EXAMPLE 7

### In Vivo Tumor Experiment with Human BxPC-3 Pancreatic Cancer Cells

In Examples 3, 4, 5 and 6, AR40A746.2.3 demonstrated efficacy as a whole antibody. To determine if efficacy could be maintained as an antibody fragment, AR40A746.2.3 and AR40A746.2.3 F(ab′)₂ were tested in an established BxPC-3 pancreatic xenograft model. AR40A746.2.3 was produced and purified as outlined in Example 2. Purified AR40A746.2.3 was subsequently cleaved by pepsin and/or ficin digestion in order to produce the F(ab′)₂ molecule. Separation of the fragments was performed using size exclusion Amicon centrifugal units (50,000 kDa molecular weight cut off) and/or Protein A chromatography.

With reference to FIGS. 12 and 13, 8 to 10 week old female SCID mice were implanted with 5 million human pancreatic cancer cells (BxPC-3) in 100 microliters PBS solution injected subcutaneously in the scruff of the neck. The mice were randomly divided into 3 treatment groups of 9 when the average mouse tumor volume reached approximately 100 mm³. On day 43 after implantation, 10 mg/kg of AR40A746.2.3 test antibody or buffer control was administered intraperitoneally to each cohort in a volume of 300 microliters after dilution from the stock concentration with a diluent that contained 2.7 mM KCl, 1 mM KH₂PO₄, 137 mM NaCl and 20 mM Na₂HPO₄, three time per week for total 10 doses. 13.3 mg/kg of AR40A746.2.3 F(ab′)₂ was administrated daily intraperitoneally for a total of 19 doses. Tumor growth was measured about every 7 days with calipers. Body weights of the animals were recorded at the same time as tumor measurement. All animals were euthanized according to CCAC guidelines at the end of the study once they had reached endpoint.

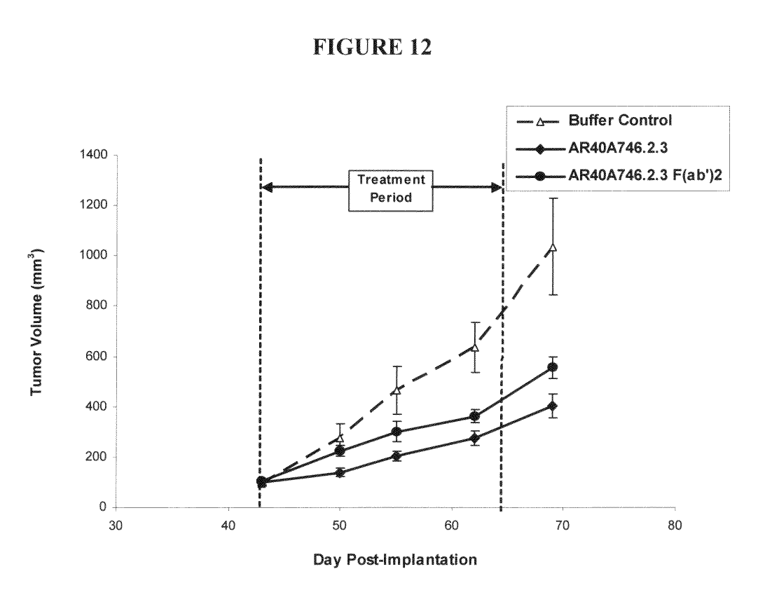

Both AR40A746.2.3 and AR40A746.2.3 F(ab′)₂ reduced tumor growth in the BxPC-3 in vivo established model of human pancreatic cancer. Treatments with Arius antibody AR40A746.2.3 and AR40A746.2.3 F(ab′)₂ significantly reduced the growth of BxPC-3 tumors by 67.6 percent (p<0.0011, t-test) and 51.7 percent (p<0.0098, t-test), respectively, compared to the buffer-treated group, as determined on day 69, 5 days after the last dose of antibody (FIG. 12).

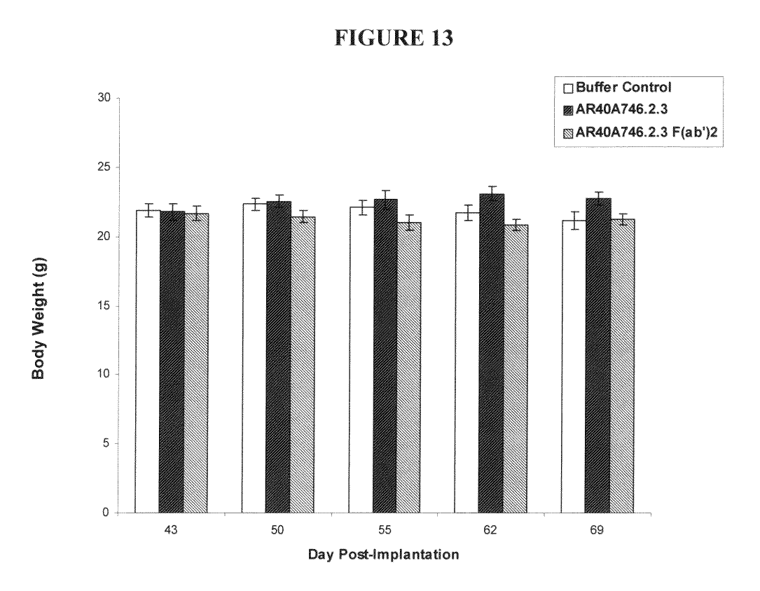

There were no clinical signs of toxicity throughout the study. Body weight measured at weekly intervals was a surrogate for well-being and failure to thrive. The mean body weight remained approximately the same in all groups over the duration of the study (FIG. 13). There were no significant differences between the groups at the end of the treatment period.

In summary, both AR40A746.2.3 and AR40A746.2.3 F(ab′)₂ were well-tolerated and significantly decreased the tumor burden in this human pancreatic cancer xenograft model.

### EXAMPLE 8

### In Vivo Tumor Experiment with Human BxPC-3 Pancreatic Cancer Cells

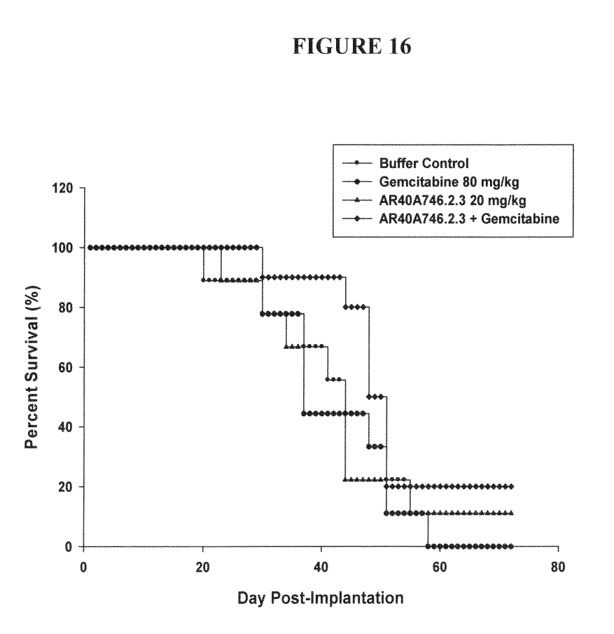

In Examples 3, 4, 6 and 7, AR40A746.2.3 demonstrated in vivo activity against xenograft models of human pancreatic cancer. To compare this activity with the clinically relevant chemotherapeutic agent, gemcitabine and to determine if the activity of the antibody could be enhanced in chemotherapeutic-antibody combinations, AR40A746.2.3 and gemcitabine were used alone and in combination in an established human BxPC-3 pancreatic cancer xenograft model. With reference to FIGS. 14, 15, 16, 17, 18 and 19, 7 to 8 week old female athymic nude were implanted subcutaneously with a BxPC-3 tumor fragment (1 mm³; the pancreatic BxPC-3 cancer cell line was maintained in athymic nude mice by serial passage) into the right flank. Tumors were monitored twice weekly and then daily as their volumes approached 80-120 mm³. On day 1 of the study, the animals were sorted into 6 treatment groups of 9-10 with tumor sizes of 62.5-126.0 mm³ and with group mean tumor sizes of 86-87.3 mm³. All agents were administrated intraperitoneally. AR40A746.2.3 test antibody at 20 mg/kg or buffer control was given three times per week for three weeks and was administered to each cohort in a volume of 200 microliters after dilution from the stock concentration with a diluent that contained 2.7 mM KCl, 1 mM KH₂PO₄, 137 mM NaCl and 20 mM Na₂HPO₄. Gemcitabine was given once daily on days 1, 4, 7 and 10. The control group mice received the PBS buffer, 3×/week for 3 weeks. Groups 2 and 3 received gemcitabine monotherapies at 160 and 80 mg/kg, respectively. Group 4 received AR40A746.2.3 monotherapy. Group 5 and 6 received gemcitabine at 160 and 80 mg/kg, respectively, in combination with AR40A746.2.3. Tumor growth was measured once every 3-4 days with calipers. The treatment was completed after 9 doses of antibody and 4 doses of gemcitabine. The endpoint volume for tumor growth was 1000 mm³. Treatment results for antibody-treated versus vehicle-treated groups were presented as (i) percent tumor growth delay (TGD), which is defined as the percent increase in the median time to endpoint (TTE), and (ii) percent tumor growth inhibition (TGI), which is defined as the decrease in the median tumor volume. Body weights of the animals were recorded at the same time as tumor measurement. All animals were euthanized according to CCAC guidelines at the end of the study once they had reached endpoint.

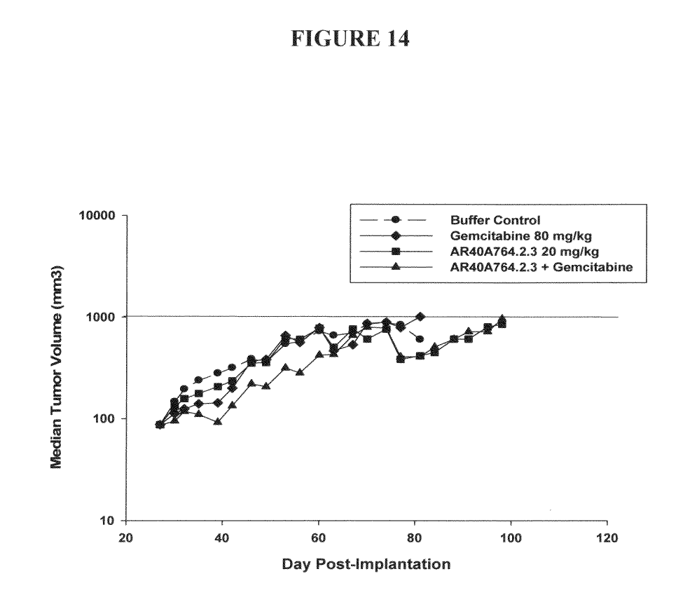

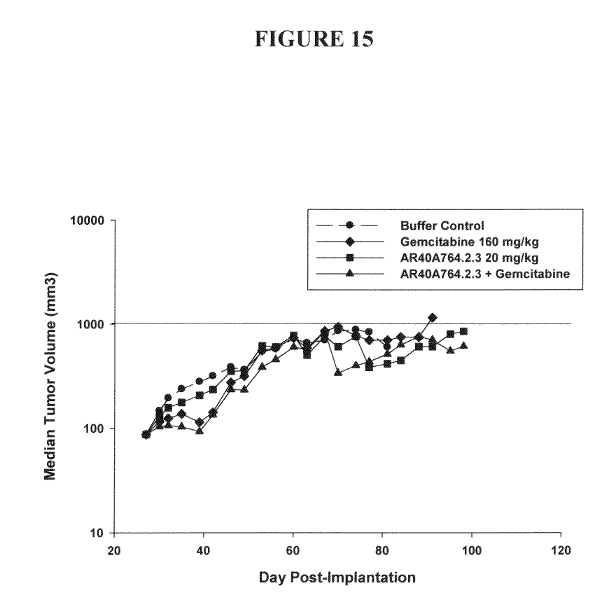

AR40A746.2.3 monotherapy demonstrated zero percent TGD, but yielded one 72-day survivor with an 850-mm³ tumor. Gemcitabine produced 9 percent and zero percent TGD at 160 and 80 mg/kg, respectively, and yielded no 72-day survivors. Combinations of AR40A746.2.3 with 160 and 80 mg/kg gemcitabine yielded 9 percent and 22 percent TGD, respectively. The high-dose combination, however, yielded two 72-day survivors with a median tumor volume of 612 mm³, as well as two animals with TTE values of more than 58 days. The low-dose combination yielded one survivor with a median tumor volume of 550-mm³, as well as one animal with a TTE of 69.5 days. Neither combination treatment achieved statistically significant activity due, in part, to the variable tumor growth rate in the vehicle-treated tumor control (FIGS. 14 and 15).

Both combinations inhibited median tumor growth from day 1 until day 13. Analysis of tumor volumes on day 13 indicates that 160 and 80 mg/kg gemcitabine monotherapies produced a significant 27 percent and 56 percent TGI (p<0.05, Mann-Whitney U-test), while AR40A746.2.3 monotherapy demonstrated an insignificant 16 percent TGI. AR40A746.2.3 at 20 mg/kg in combination with 160 or 80 mg/kg gemcitabine yielded highly significant 53 percent and 56 percent TGI (p<0.001, Mann-Whitney U-test) (FIGS. 14 and 15).

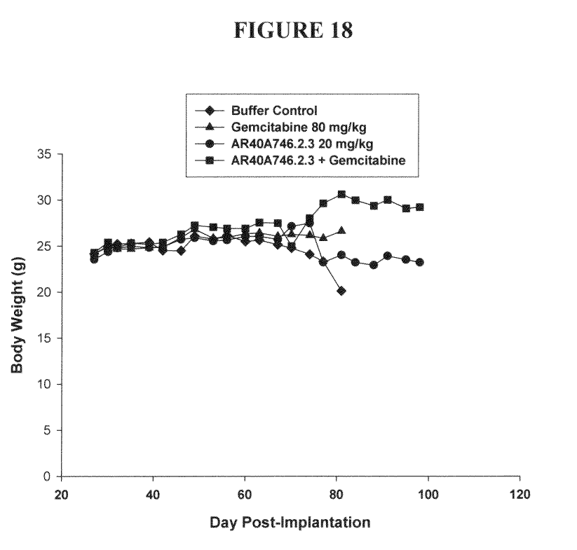

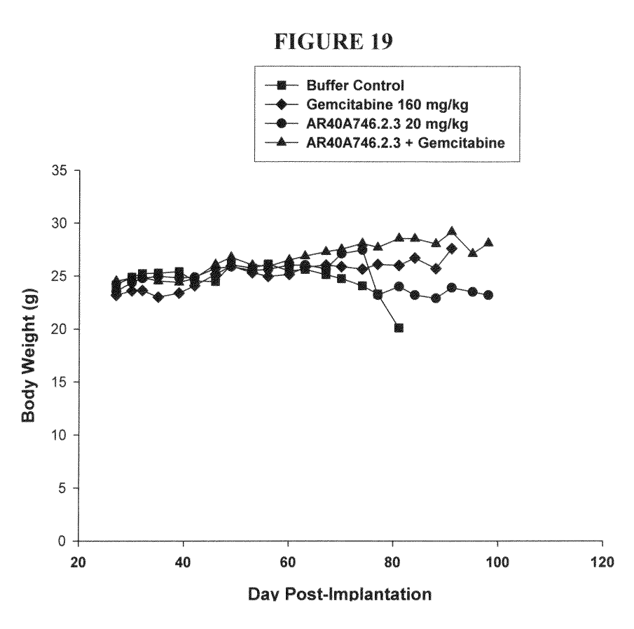

There were no obvious clinical signs of toxicity throughout the study. Body weight measured at weekly intervals was a surrogate for well being and failure to thrive. Negligible (<1 percent) maximum group mean body weight losses occurred in group 2 (gemcitabine monotherapy at 160 mg/kg) and group 5 (AR40A746.2.3. in combination with gemcitabine at 160 mg/kg). There were no significant differences between the groups during the treatment period (FIGS. 18 and 19).

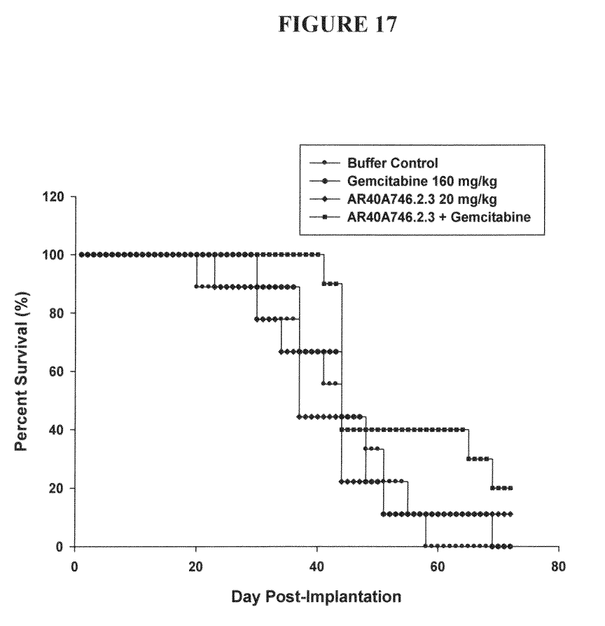

In summary, logrank analyses of TTE values indicate that AR40A746.2.3 or gemcitabine monotherapy or their combinations produced activities against BxPC-3 pancreatic cancer xenografts. On day 13, every antibody or chemotherapy or their combination except AR40A746.2.3 monotherapy, produced statistically significant TGI. The results demonstrate a dose-dependent trend toward therapeutic activity: 40 percent and 20 percent of the animals treated with the 160 and 80 mg/kg gemcitabine/AR40A746.2.3 combinations, respectively, experienced substantially prolonged survival, whereas, the percentage of monotherapy-treated mice that experienced substantially prolonged survival was 11-12.5 percent (FIGS. 17 and 18).

### EXAMPLE 9

### In Vivo Tumor Experiment with Human MDA-MB-231 Cancer Cells

With reference to FIGS. 20 and 21, 8 to 10 week old female SCID mice were implanted with 5 million human breast adenocarcinoma cells (MDA-MB-231) in 100 microliters PBS solution, injected subcutaneously in the right flank of each mouse. The mice were randomly divided into 2 treatment groups of 10. One day after implantation, 20 mg/kg of AR40A746.2.3 test antibody or buffer control was administered intraperitoneally to each cohort in a volume of 300 microliters, after dilution from the stock concentration with a PBS buffer solution. The antibody and control samples were then administered once per week for 7 weeks. Tumor growth was measured once a week with calipers. The treatment was completed after 8 doses of antibody. Body weights of the animals were recorded when tumors were measured for the duration of the study. At the end of the study all animals were euthanized according to CCAC guidelines when reaching endpoint.

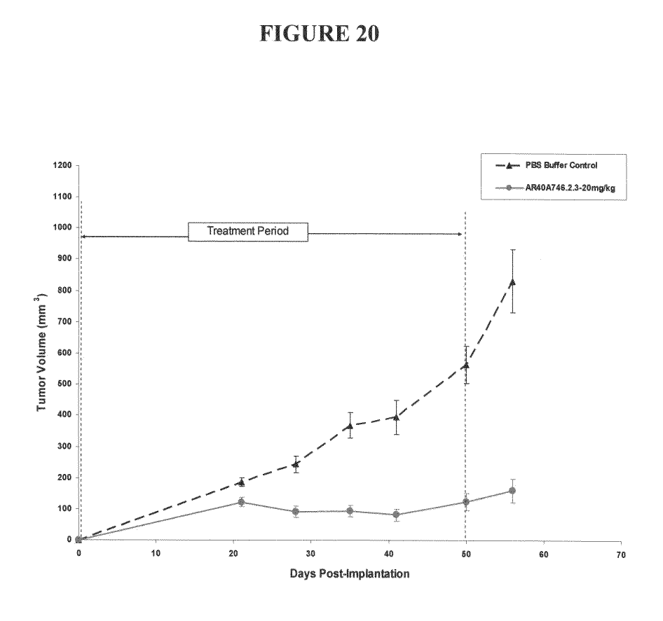

AR40A746.2.3 significantly inhibited tumor growth in the MDA-MB-231 in vivo prophylactic model of human breast adenocarcinoma. Treatment with ARIUS antibody AR40A748.2.3 reduced the growth of MDA-MB-231 tumors by 80.6 percent (p<0.00001, t-test) compared to the buffer treated group, as determined on day 56, 6 days after the last dose of antibody was administered (FIG. 20).

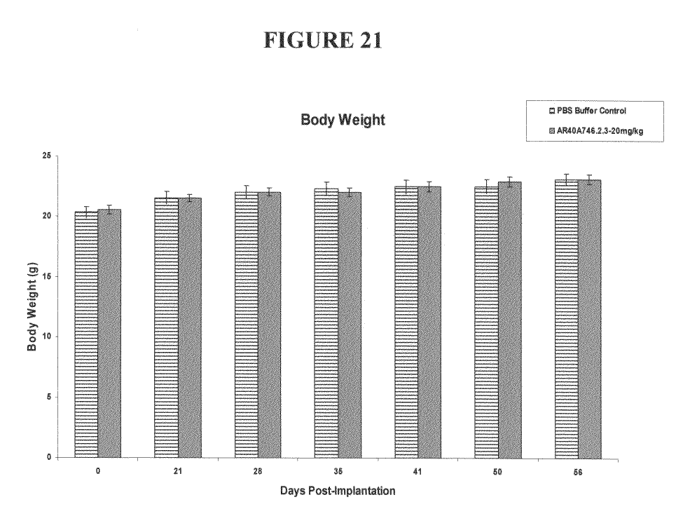

There were no obvious clinical signs of toxicity throughout the study. Body weight measured at weekly intervals was a surrogate for well-being and failure to thrive. The mean body weight increased in all groups over the duration of the study (FIG. 21). The mean weight gain between day 0 and day 56 was +2.76 g (+13.6 percent) in the control group and +2.59 (+12.6 percent) in the AR40A746.2.3-treated group. There were no significant differences between groups during the treatment period.

In summary, AR40A746.2.3 was well-tolerated and significantly inhibited tumor growth in this human breast adenocarcinoma xenograft model at day 56.

### EXAMPLE 10

### Human Normal Tissues

IHC studies were conducted to characterize the AR40A746.2.3 antigen distribution in human normal tissues. Fifty-nine human normal tissues represented on a tissue array (Imgenex, San Diego, Calif.) were tested. Previous experiments were conducted to optimize the IHC binding conditions of the antibody. Tissue sections were deparaffinized by drying in an oven at 58° C. for 1 hour and dewaxed by immersing in xylene 5 times for 4 minutes each in Coplin jars. Following treatment through a series of graded ethanol washes (100 to 75 percent) the sections were re-hydrated in water. The slides were immersed in 10 mM citrate buffer at pH 6 (Dako, Toronto, Ontario) then microwaved at high, medium, and low power settings for 5 minutes each and finally immersed in cold PBS. Slides were then immersed in 3 percent hydrogen peroxide solution for 6 minutes, washed with PBS three times for 5 minutes each, dried and incubated with Universal blocking solution (Dako, Toronto, Ontario) for 5 minutes at room temperature. AR40A746.2.3 or isotype control antibody (directed towards Aspergillus niger glucose oxidase, an enzyme which is neither present nor inducible in mammalian tissues; Dako, Toronto, Ontario) was diluted in antibody dilution buffer (Dako, Toronto, Ontario) to its working concentration (5 micrograms/mL for each antibody) and incubated for 1 hour at room temperature in humidified chamber. Monoclonal mouse anti-actin (Dako, Toronto, Ontario) was diluted to its working concentration of 2 micrograms/mL. The slides were washed with PBS 3 times for 5 minutes each. Immunoreactivity of the primary antibodies was detected/visualized with HRP conjugated secondary antibodies as supplied (Dako Envision System, Toronto, Ontario) for 30 minutes at room temperature. Following this step the slides were washed with PBS 3 times for 5 minutes each and a color reaction developed by adding DAB (3,3′-diaminobenzidine tetrahydrochloride, Dako, Toronto, Ontario) chromogen substrate solution for immunoperoxidase staining for 10 minutes at room temperature. Washing the slides in tap water terminated the chromogenic reaction. Following counterstaining with Meyer's Hematoxylin (Sigma Diagnostics, Oakville, Ontario), the slides were dehydrated with graded ethanols (75 to 100 percent) and cleared with xylene. Using mounting media (Dako Faramount, Toronto, Ontario) the slides were coverslipped. Slides were microscopically examined using an Axiovert 200 (Zeiss Canada, Toronto, Ontario) and digital images acquired and stored using Northern Eclipse Imaging Software (Mississauga, Ontario). Results were read, scored and interpreted by a histopathologist.

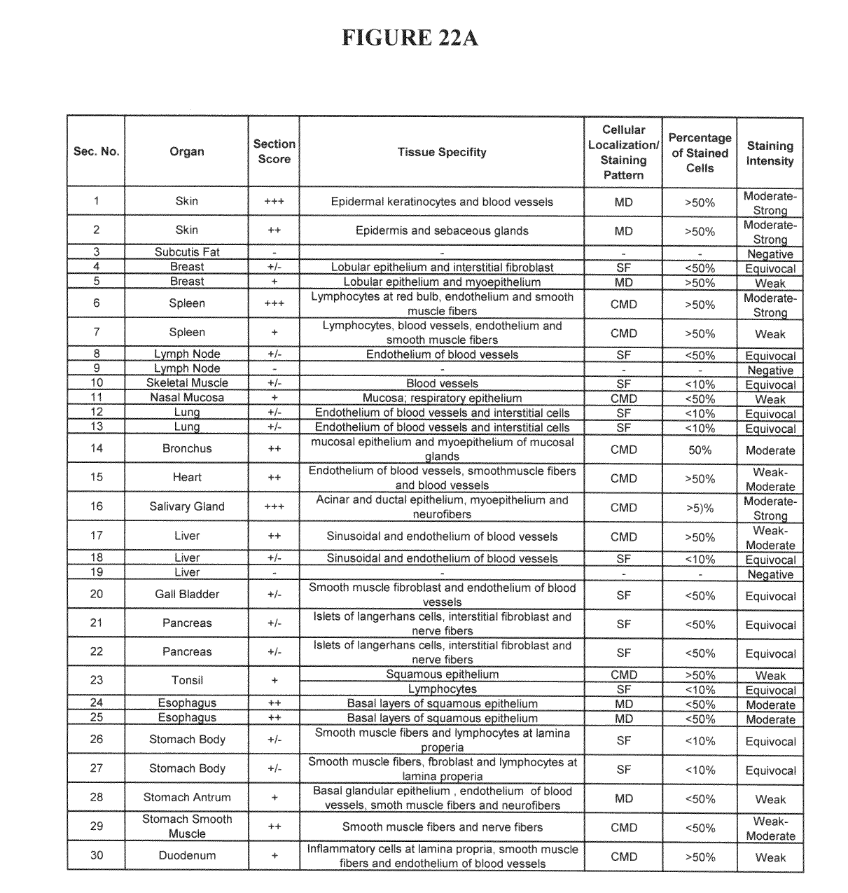

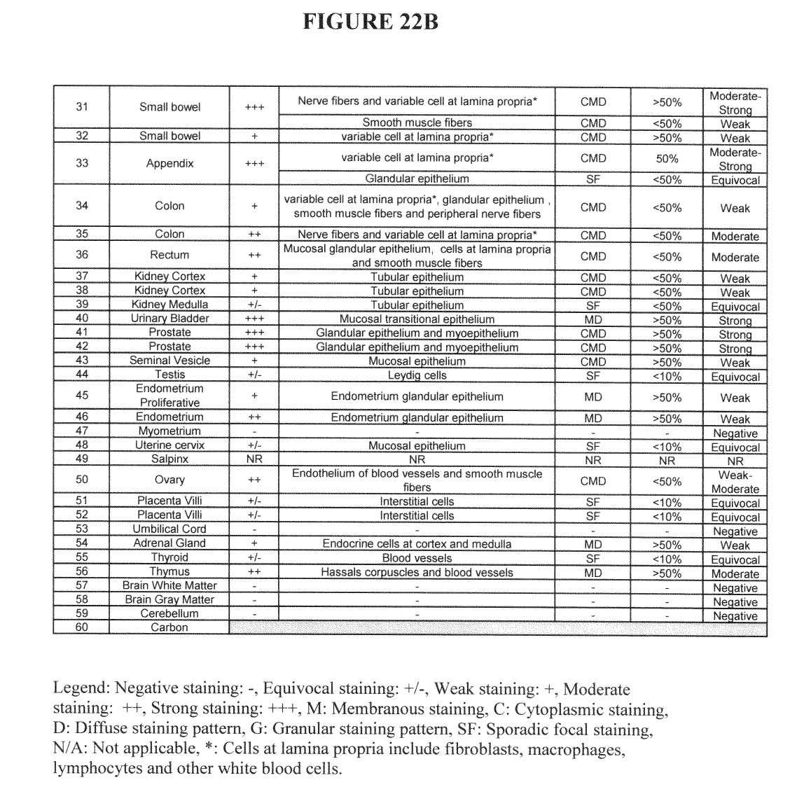

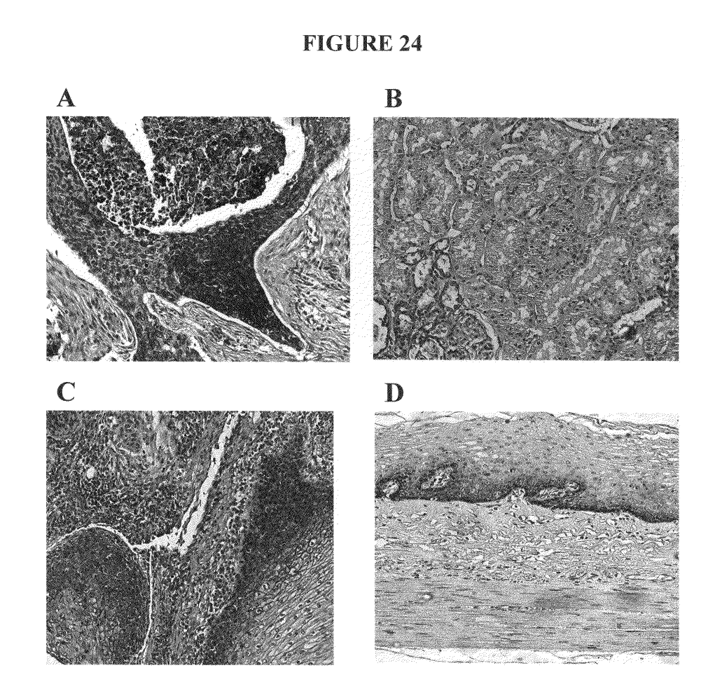

Binding of AR40A746.2.3 to 59 human normal tissue samples was performed using a human, normal tissue array (Imgenex, San Diego, Calif.). FIGS. 22A-22B summarize the results of AR40A746.2.3 staining of various human normal tissues. The AR40A746.2.3 antibody showed binding predominantly to epithelial tissues (FIG. 24, Panels B and D). In addition, binding to connective, muscular and peripheral nerve tissues was observed. Cellular localization was predominantly membranous. Cytoplasmic staining was observed in the cells of some of the tissues. The anti-actin positive control antibody showed specific binding to muscular tissues. The IgG isotype negative control showed no binding to any of the tested tissues.

### EXAMPLE 11

### Human Tumor Tissues

IHC studies were conducted to characterize the AR40A746.2.3 antigen prevalence in human cancers. Fifty-nine human tumor tissues from one array (Imgenex, San Diego, Calif.) and another 12 tumor tissues and representative normal tissues from another array (Tri Star, Rockville, Md.) were tested. Previous experiments were conducted to optimize the IHC binding conditions of the antibody. Tissue sections were deparaffinized by drying in an oven at 58° C. for 1 hour and dewaxed by immersing in xylene 5 times for 4 minutes each in Coplin jars. Following treatment through a series of graded ethanol washes (100 to 75 percent), the sections were re-hydrated in water. The slides were immersed in 10 mM citrate buffer at pH 6 (Dako, Toronto, Ontario) then microwaved at high, medium, and low power settings for 5 minutes each and finally immersed in cold PBS. Slides were then immersed in 3 percent hydrogen peroxide solution for 6 minutes, washed with PBS three times for 5 minutes each, dried and incubated with Universal blocking solution (Dako, Toronto, Ontario) for 5 minutes at room temperature. AR40A746.2.3 or isotype control antibody (directed towards Aspergillus niger glucose oxidase, an enzyme which is neither present nor inducible in mammalian tissues; Dako, Toronto, Ontario) were diluted in antibody dilution buffer (Dako, Toronto, Ontario) to their working concentration (5 micrograms/mL for each antibody) and incubated for 1 hour at room temperature in humidified chamber. Anti-Action was diluted to its working concentration of 2 micrograms/mL. The slides were washed with PBS 3 times for 5 minutes each. Immunoreactivity of the primary antibodies was detected/visualized with HRP conjugated secondary antibodies as supplied (Dako Envision System, Toronto, Ontario) for 30 minutes at room temperature. Following this step the slides were washed with PBS 3 times for 5 minutes each and a color reaction developed by adding DAB (3,3′-diaminobenzidine tetrahydrochloride, Dako, Toronto, Ontario) chromogen substrate solution for immunoperoxidase staining for 10 minutes at room temperature. Washing the slides in tap water terminated the chromogenic reaction. Following counterstaining with Meyer's Hematoxylin (Sigma Diagnostics, Oakville, Ontario), the slides were dehydrated with graded ethanols (75 to 100 percent) and cleared with xylene. Using mounting media (Dako Faramount, Toronto, Ontario) the slides were coverslipped. Slides were microscopically examined using an Axiovert 200 (Zeiss Canada, Toronto, Ontario) and digital images acquired and stored using Northern Eclipse Imaging Software (Mississauga, Ontario). Results were read, scored and interpreted by a histopathologist.

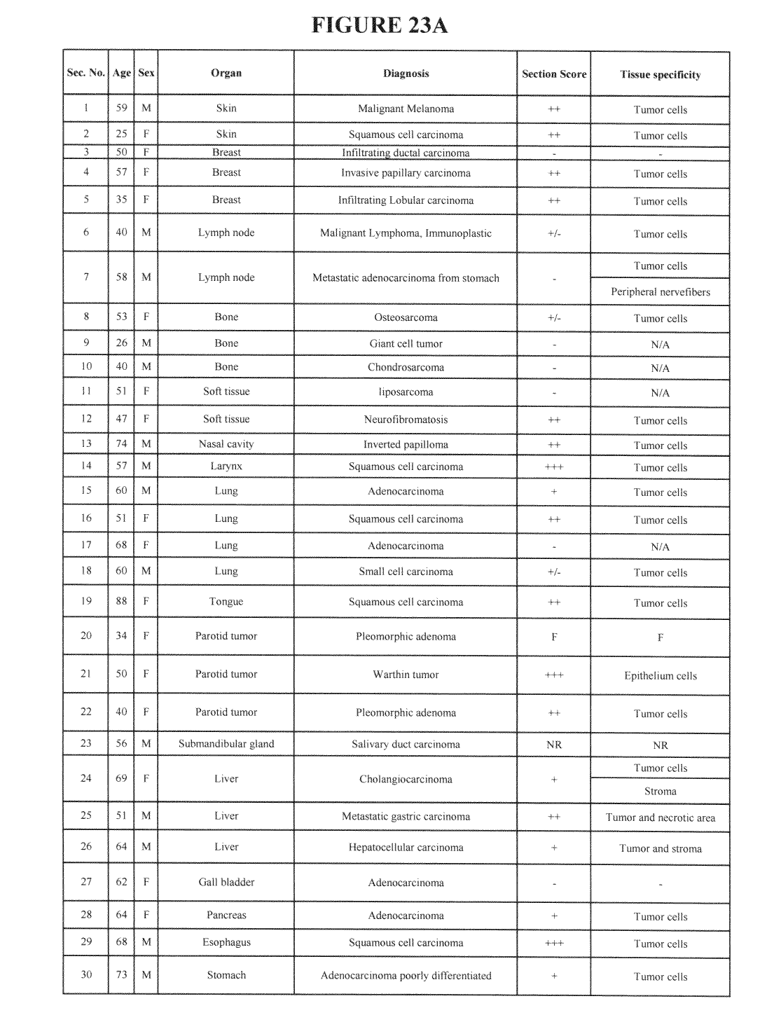

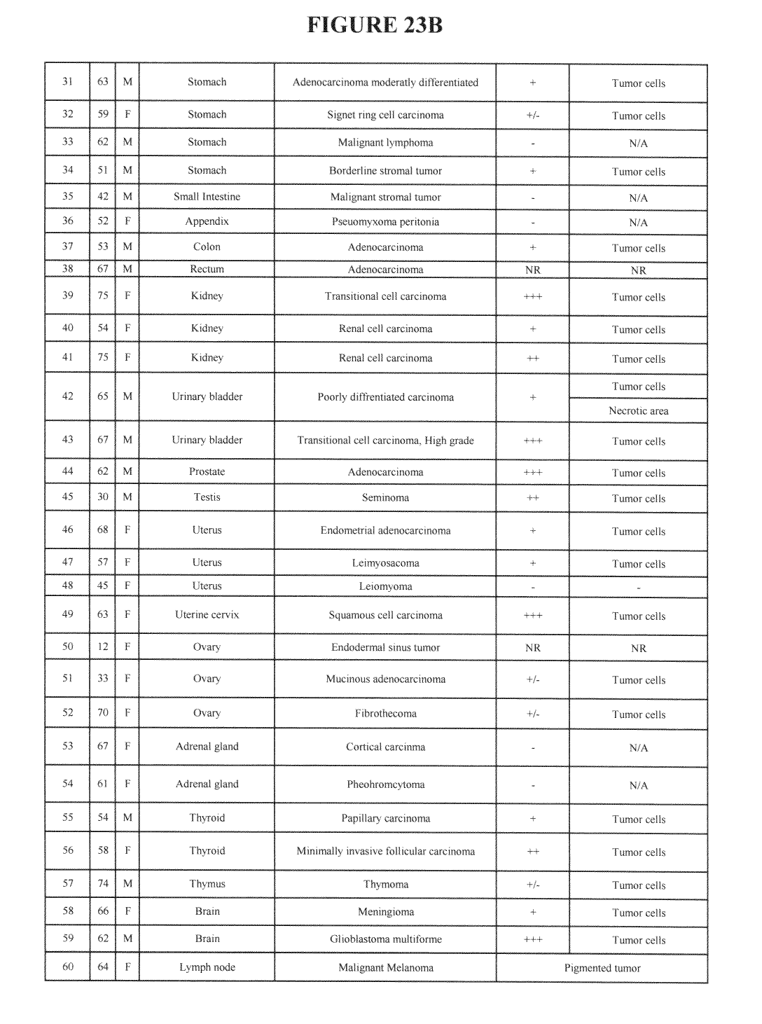

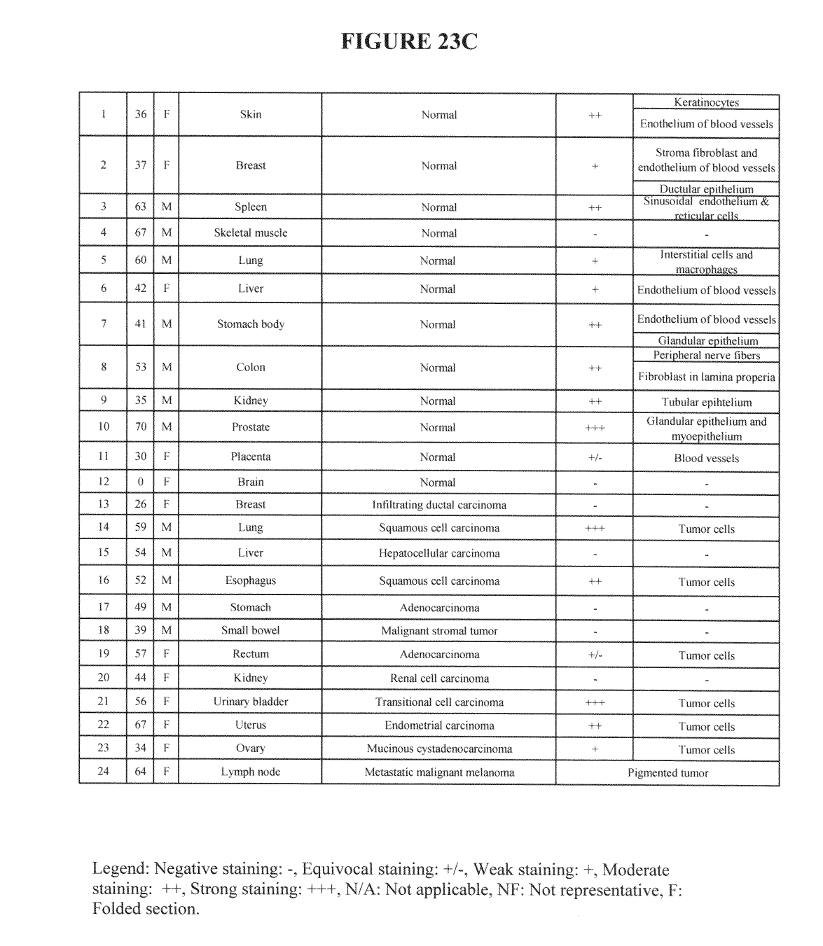

FIGS. 23A-23C summarizes the results of the binding of the antibody to various human tumor tissues from two different tissue arrays. Sixty-six tumor samples were interpretable. There was moderate to strong staining of the tumor cells in 25/66 (38 percent) of tested tumors including; malignant melanoma, squamous cell carcinoma of various organs (including the esophagus), transitional cell carcinoma of the kidney and bladder, renal cell carcinoma of kidney, adenocarcinoma of prostate, glioblastoma multiformi of brain, thyroid follicular carcinoma, endometrial carcinoma and metastatic gastric carcinoma to liver (FIG. 24, Panels A and C). Weak and equivocal staining was observed in 23/66 (35 percent) of the tested tumor tissue samples. The cellular localization was predominantly membranous, cytoplasmic staining was also observed in the tumor cells of some of the tissues. In the normal tissues, the antibody showed binding predominantly to epithelial tissues which is consistent with the data outlined in Example 9. No binding to skeletal muscle or brain was observed. There was over expression of the AR40A746.2.3 epitope in tumor versus normal tissues including the lung and brain. The anti-actin positive control antibody showed specific binding to muscular tissues. The IgG isotype negative control showed no binding to any of the tested tissues. These results demonstrate that the AR40A746.2.3 epitope is found on cancer cells and is over expressed in some tumor tissues.

### EXAMPLE 12

### Pancreatic Human Tumor Tissue

IHC studies were conducted to further characterize the AR40A746.2.3 antigen prevalence in human pancreatic cancers. Thirty-three pancreatic cancer tissues and 4 representative non neoplastic pancreatic tissues were tested from a human tissue micro array (Petagen, ISU ABXIS Co, Seoul, South Korea). The cancer tissue samples were in duplicates for each case. The final score represents the highest predominant staining intensity from both samples of the tumor. Previous experiments were conducted to optimize the IHC binding conditions of the antibody. Tissue sections were deparaffinized by drying in an oven at 58° C. for 1 hour and dewaxed by immersing in xylene 5 times for 4 minutes each in Coplin jars. Following treatment through a series of graded ethanol washes (100 to 75 percent) the sections were re-hydrated in water. The slides were immersed in 10 mM citrate buffer at pH 6 (Dako, Toronto, Ontario) then microwaved at high, medium, and low power settings for 5 minutes each and finally immersed in cold PBS. Slides were then immersed in 3 percent hydrogen peroxide solution for 6 minutes, washed with PBS three times for 5 minutes each, dried, incubated with Universal blocking solution (Dako, Toronto, Ontario) for 5 minutes at room temperature. AR40A746.2.3 or isotype control antibody (directed towards Aspergillus niger glucose oxidase, an enzyme which is neither present nor inducible in mammalian tissues; Dako, Toronto, Ontario) was diluted in antibody dilution buffer (Dako, Toronto, Ontario) to its working concentration (5 micrograms/mL for each antibody) and incubated for 1 hour at room temperature in humidified chamber. Anti-actin was diluted to its working concentration of 2 micrograms/mL. The slides were washed with PBS 3 times for 5 minutes each. Immunoreactivity of the primary antibodies was detected/visualized with HRP conjugated secondary antibodies as supplied (Dako Envision System, Toronto, Ontario) for 30 minutes at room temperature. Following this step the slides were washed with PBS 3 times for 5 minutes each and a color reaction developed by adding DAB (3,3′-diaminobenzidine tetrahydrochloride, Dako, Toronto, Ontario) chromogen substrate solution for immunoperoxidase staining for 10 minutes at room temperature. Washing the slides in tap water terminated the chromogenic reaction. Following counterstaining with Meyer's Hematoxylin (Sigma Diagnostics, Oakville, Ontario), the slides were dehydrated with graded ethanols (75 to 100 percent) and cleared with xylene. Using mounting media (Dako Faramount, Toronto, Ontario) the slides were coverslipped. Slides were microscopically examined using an Axiovert 200 (Zeiss Canada, Toronto, Ontario) and digital images acquired and stored using Northern Eclipse Imaging Software (Mississauga, Ontario). Results were read, scored and interpreted by a histopathologist.

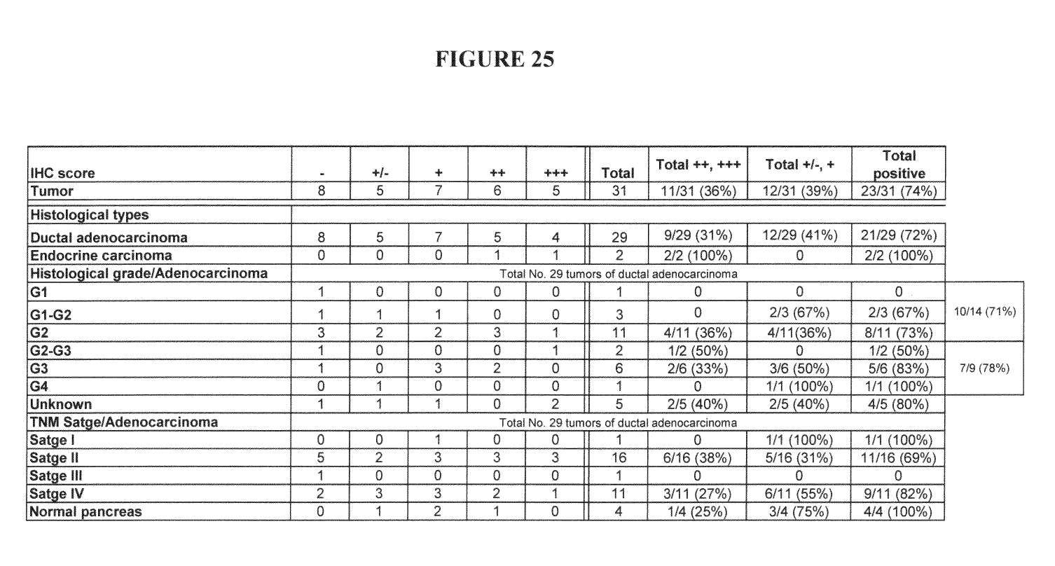

FIG. 25 summarizes the results of the binding of the antibody to pancreatic cancers in a tissue array. Thirty-one pancreatic tumor tissue samples (including 29 adenocarcinomas and 2 endocrine carcinomas) and 4 normal tissue samples were interpretable. In total, there was moderate to strong staining of the tumor cells in 11/31 (36 percent) and equivocal to weak in 12/31 (39 percent) of the tested tumor tissues. For the adenocarcinomas, there was moderate to strong staining of the tumor cells in 9/29 (31 percent) and equivocal to weak in 12/29 (41 percent) of the tested tumor tissues. For endocrine tumors, there was moderate to strong staining in both of the tested samples (2/2). There was a trend towards higher binding with higher histological grades (G2-3, G3 and G4). There was no obvious correlation of the antibody binding with TNM tumor stages. The cellular localization was predominantly membranous, cytoplasmic staining was also observed in tumor cells of some of the tested tissues.

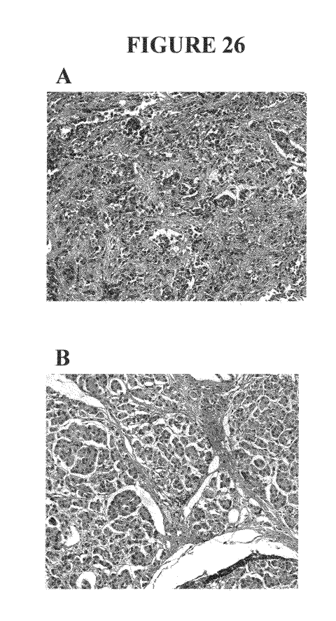

In the 4 tested non neoplastic pancreatic tissues, there was moderate to strong staining in 1/4 (25 percent) and equivocal to weak in 3/4 (75 percent) of the tested tumor tissues. The binding was predominantly to epithelial tissues. The anti-actin positive control antibody showed specific binding to muscular tissues. The IgG isotype negative control showed no binding to any of the tested tissues. In comparing the intensity of the binding of AR40A746.2.3 to pancreatic cancers and non neoplastic pancreatic tissues, there was over expression of the epitope targeted by AR40A746.2.3 in neoplastic (FIG. 26A) versus non neoplastic human pancreatic tissues (FIG. 26B). These results demonstrate that the epitope recognized by AR40A746.2.3 is expressed on pancreatic cancers and is over expressed on tumor versus normal pancreatic tissue.

### EXAMPLE 13

### Cross Reactivity to Normal Human and Other Species Tissues

IHC studies were conducted to evaluate the cross reactivity of AR40A746.2.3 to non human species tissues in order to find suitable preclinical toxicology model(s). All tissues used were formalin fixed paraffin embedded. The binding of AR40A746.2.3 to 8 normal tissues of cynomolgus and rhesus monkey (Biochain, CA, USA) and 10 normal tissues of rabbit, rat, mouse and sheep (Zymed laboratories Inc, CA, USA) was performed using tissue micro arrays. Previous experiments were conducted to optimize the IHC binding conditions of the antibody. Tissue sections were deparaffinized by drying in an oven at 58° C. for 1 hour and dewaxed by immersing in xylene 5 times for 4 minutes each in Coplin jars. Following treatment through a series of graded ethanol washes (100 to 75 percent) the sections were re-hydrated in water. The slides were immersed in 10 mM citrate buffer at pH 6 (Dako, Toronto, Ontario) then microwaved at high, medium, and low power settings for 5 minutes each and finally immersed in cold PBS. Slides were then immersed in 3 percent hydrogen peroxide solution for 6 minutes, washed with PBS three times for 5 minutes each, dried and incubated with Universal blocking solution (Dako, Toronto, Ontario) for 5 minutes at room temperature. AR40A746.2.3, monoclonal mouse anti-actin (Dako, Toronto, Ontario) or isotype control antibody (directed towards Aspergillus niger glucose oxidase, an enzyme which is neither present nor inducible in mammalian tissues; Dako, Toronto, Ontario) was diluted in antibody dilution buffer (Dako, Toronto, Ontario) to its working concentration (5 micrograms/mL) except anti-actin which was diluted to 2 micrograms/mL and incubated for 1 hour at room temperature in a humidified chamber. The slides were washed with PBS 3 times for 5 minutes each. Immunoreactivity of the primary antibodies was detected/visualized with HRP conjugated secondary antibodies as supplied (Dako Envision System, Toronto, Ontario) for 30 minutes at room temperature. Following this step the slides were washed with PBS 3 times for 5 minutes each and a color reaction developed by adding DAB (3,3′-diaminobenzidine tetrahydrochloride, Dako, Toronto, Ontario) chromogen substrate solution for immunoperoxidase staining for 10 minutes at room temperature. Washing the slides in tap water terminated the chromogenic reaction. Following counterstaining with Meyer's Hematoxylin (Sigma Diagnostics, Oakville, Ontario), the slides were dehydrated with graded ethanols (75 to 100 percent) and cleared with xylene. Using mounting media (Dako Faramount, Toronto, Ontario) the slides were coverslipped. Slides were microscopically examined using an Axiovert 200 (Zeiss Canada, Toronto, Ontario) and digital images acquired and stored using Northern Eclipse Imaging Software (Mississauga, Ontario). Results were read, scored and interpreted by a histopathologist.

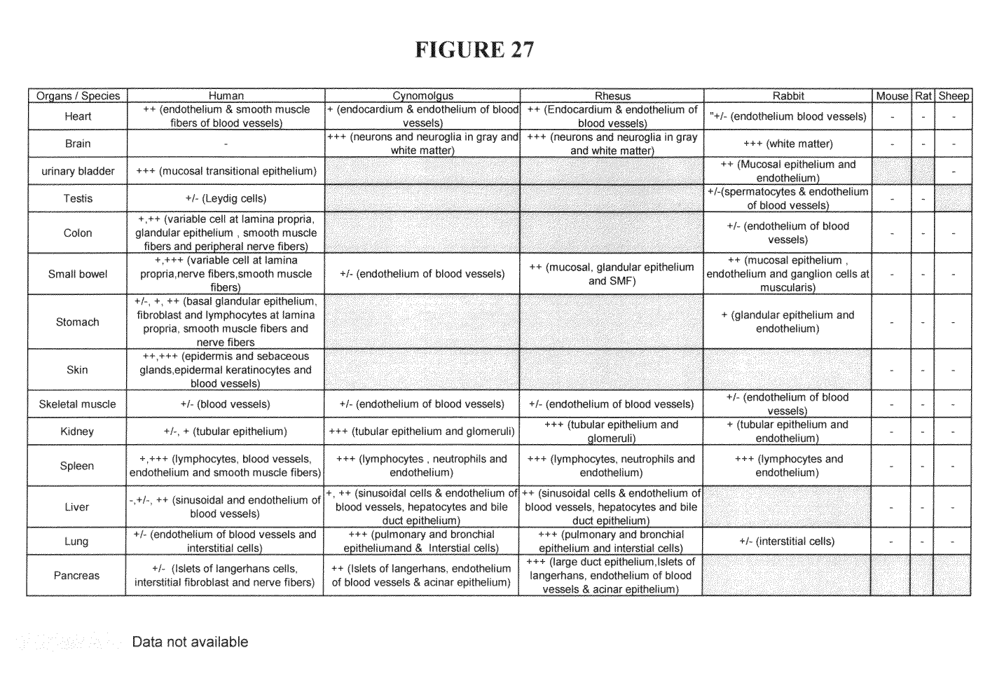

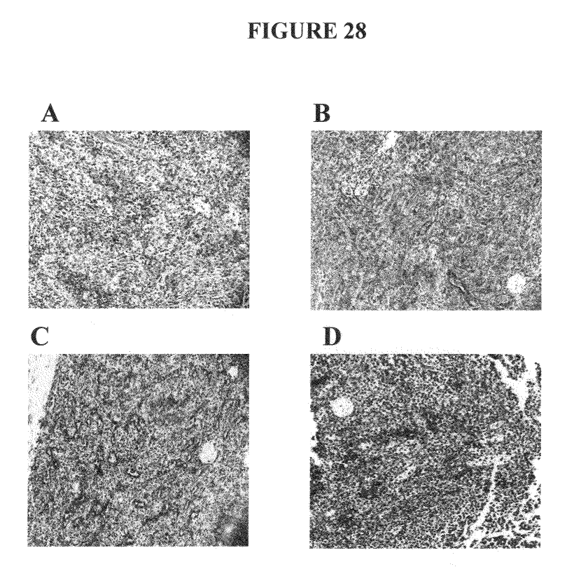

Some of the tissues were not representative and consequently were not included in the final interpretation. FIG. 27 presents a summary of the results of AR40A746.2.3 binding to cynomolgus, rhesus, rabbit, mouse, rat and sheep normal tissues compared to the binding of the antibody to previously tested normal human tissues (Example 9). AR40A746.2.3 antibody showed binding predominantly to the epithelial tissues, inflammatory cells and neural tissues of human (FIG. 28A), cynomolgus monkey (26B), rhesus monkey (26C) and rabbit (26D). No binding was observed to the mouse, rat or sheep tissues. The anti-actin positive control antibody showed specific binding to muscular tissues. The IgG isotype negative control showed no binding to any of the interpreted tissues. AR40A746.2.3 therefore cross reacts with the cynomolgus monkey, rhesus monkey and rabbit normal tissues in a similar manner as to the human normal tissues.

### EXAMPLE 14

### Identification of Antigen Bound by AR40A746.2.3

### 1. Immunoprecipitation

The identification of the antigen for AR40A746.2.3 was carried out by isolating the cognate ligand through immunoprecipitation of solubilized lysate from BxPC-3 cells. One hundred microliters of Protein G Dynabeads (Invitrogen, Burlington, Ontario) were washed 3 times with 1 mL of 0.1 M sodium phosphate buffer pH 6.0. One hundred micrograms of AR40A746.2.3 in a total volume of 100 microliters 0.1 M sodium phosphate pH 6.0 was added to the washed beads. The mixture was incubated for 1 hour with end-over-end mixing. Unbound antibody was removed and the AR40A746.2.3 coated beads were washed 3 times with 0.5 mL 0.1 M sodium phosphate pH 7.4 containing 0.1 percent Tween-20. The AR40A746.2.3 coated beads were washed 2 times with 1 mL 0.2 M triethanolamine pH 8.2. AR40A746.2.3 was chemically crosslinked to the beads by adding 1 mL of freshly prepared 0.02 M dimethylpimelimidate in 0.2 M triethanolamine pH 8.2 and incubating with end-over-end mixing for 30 minutes. The reaction was stopped by incubating the beads with 1 mL of 0.05 M Tris pH 7.5 for 15 minutes with rotational mixing. The AR40A746.2.3 crosslinked beads were pre-eluted by incubation with 0.1 M citrate pH 3.0 for 3 minutes followed by 3 washes in 0.1 M PBS containing 0.1 percent Tween-20. A second set of antibody crosslinked beads were prepared in the same manner described using a mouse IgG1 antibody (clone 1B7.11, purified in-house) to trinitrophenol, which was used as a negative IgG1 isotype control.

The AR40A746.2.3 crosslinked beads were blocked by incubating in 0.1 percent BSA in 0.1 M sodium phosphate pH 7.4 with rotational mixing for 30 minutes at room temperature. The beads were washed three times with 0.1 M sodium phosphate pH 7.4. Five milligrams of a lysate preparation from BxPC-3 cells was incubated with the AR40A746.2.3 crosslinked beads with rotational mixing for 2 hours at room temperature. The immunocomplex bound beads were washed once with 1 mL of 1 mM KH₂PO₄, 10 mM Na₂HPO₄, 137 mM NaCl and 2.7 mM KCl containing 0.1 percent Triton X-100 followed by a second wash with 1 mL of 1 mM KH₂PO₄, 10 mM Na₂HPO₄, 637 mM NaCl and 2.7 mM KCl containing 0.1 percent Triton X-100 for 5 minutes with end-over-end mixing, followed by a final wash with 1 mL of 1 mM KH₂PO₄, 10 mM Na₂HPO₄, 137 mM NaCl and 2.7 mM KCl containing 0.1 percent Triton X-100. Fourteen microliters of non-reducing SDS-PAGE sample buffer was added to the washed immunocomplex bound beads and the sample was boiled for 5 minutes. The supernatant containing the dissociated immunocomplexes was removed and placed into a microfuge tube containing 1 microliter of 2-mercaptoethanol. The IgG1 isotype control (clone 1B7.11) crosslinked beads were incubated with BxPC-3 lysate preparation and processed in the same manner as the AR40A746.2.3 beads.

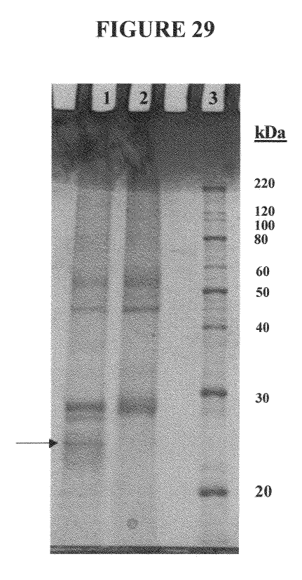

The AR40A746.2.3 immunoprecipitated protein was loaded onto a single well of a 12 percent polyacrylamide gel alongside the immunoprecipitate generated from the IgG1 isotype control (clone 1B7.11). A sample of MagicMark molecular weight markers (Invitrogen, Burlington, Ontario) was loaded in a reference lane. The polyacrylamide gel containing the immunoprecipitate samples was electrophoresed at 150 V for approximately 70 minutes. The gel was stained for approximately 17 hours with Colloidal Blue protein stain (Invitrogen, Burlington, Ontario), according to the manufacturer's directions. Presented in FIG. 29 is a photograph of the stained gel. There was a band present in the AR40A746.2.3 immunoprecipitate at approximately 25 kDa that was not present in the IgG1 isotype control immunoprecipitate. Accordingly, the area of the gel containing the 25 kDa band from the AR40A746.2.3 immunoprecipitate was excised using a glass Pasteur pipette, along with the corresponding area in the lane containing IgG1 isotype control (clone 1B7.11) immunoprecipitate.

### 2. Mass Spectrometry

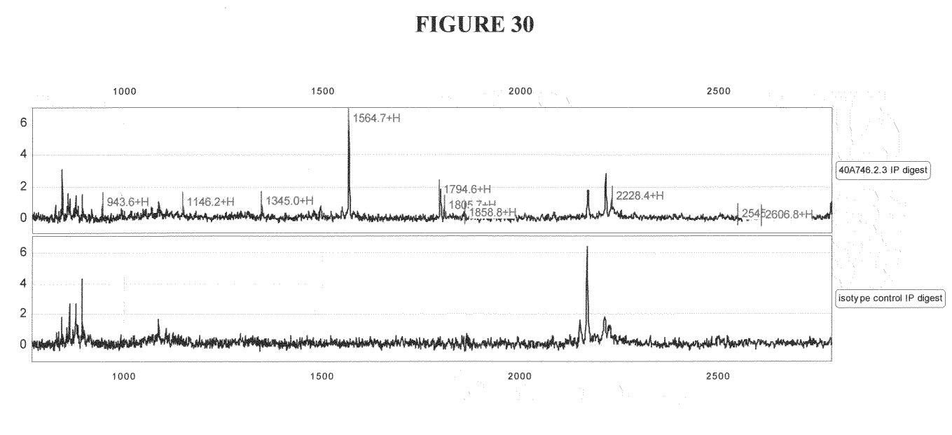

The excised gel pieces were subjected to trypsin digestion. Briefly, the gel pieces were destained and dehydrated in microfuge tubes by performing 2 washes using 50 percent methanol, 10 percent acetic acid for 30 minutes each with agitation, followed by incubation with 50 percent acetonitrile, 0.1 M ammonium bicarbonate for 1 hour with agitation. One hundred percent acetonitrile was added to the samples and incubated for 15 minutes with agitation. All liquid was removed and the gel pieces were dehydrated completely by incubation at 75° C. for 10 minutes with the tops of the microfuge tubes left open. Trypsin digestion was performed by incubating the dehydrated gel pieces with 10 microliters of freshly prepared 0.01 mg/mL activated trypsin (Pierce, Rockford, Ill.) for 15 minutes, followed by the addition of 25 mM ammonium bicarbonate. The samples were incubated for approximately 13 hours at 37° C. One microliter of each sample (containing peptides resulting from the trypsin digest) was applied to a spot on an H4 chip (Ciphergen Biosystems, Fremont, Calif.) and was allowed to dry. Half a microliter of 20 percent saturated alpha-cyano-4-hydroxy-cinnamic acid in 0.5 percent trifluoroacetic acid 50 percent acetonitrile was applied twice to each spot. Spectra for each sample were obtained on a PBS-IIc mass spectrometer (Ciphergen Biosystems, Fremont, Calif.). An overview of the spectra obtained for each sample is shown in FIG. 30. The spectra were visually scanned and peaks specific to the AR40A746.2.3 digest compared to the IgG1 isotype control (clone 1B7.11) digest were labeled. Ten distinct peaks were identified in the AR40A746.2.3 immunoprecipitate digest that were not present in the IgG1 isotype control digest. In order to accurately identify the protein immunoprecipitated by AR40A746.2.3, tandem mass spectrometry was performed on one of the peptides present in the AR40A746.2.3 tryptic digest. A second H4 chip was prepared in the same manner described above and a 1570 Da peptide present in the AR40A746.2.3 digest was analyzed by collision-induced dissociation using a Q-TOF tandem mass spectrometer in order to generate the amino acid sequence of that peptide. The amino acid sequence determined for the 1570 Da peptide was searched against Mascot peptide mapping database (Matrix Science Ltd, London, UK). A high confidence match with human CD9 was returned from the database.

### 3. Confirmation of Antigen Identity

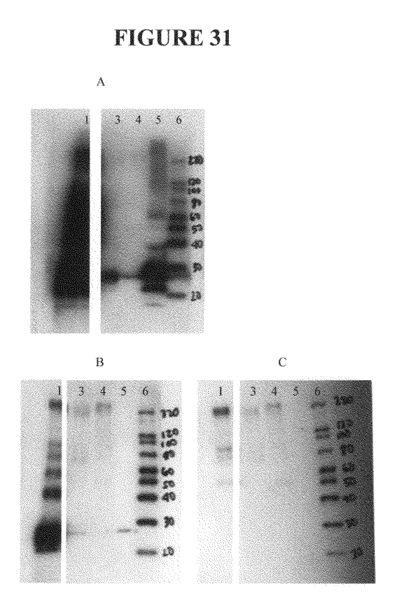

Confirmation of CD9 as the antigen target of AR40A746.2.3 was carried out by doing cross-immunoprecipitations to determine whether a known anti-CD9 antibody would react with the protein immunoprecipitated by AR40A746.2.3 and vice-versa. Antibody-crosslinked beads and immunoprecipitates were prepared in the same manner as described using the antibodies AR40A746.2.3, IgG1 isotype control (clone 1B7.11) and anti-CD9 (clone MEM-61; Abcam, Cambridge, Mass.). AR40A746.2.3 immunoprecipitate, anti-CD9 (clone MEM-61) immunoprecipitate, IgG1 isotype control (clone 1B7.11) immunoprecipitate and BxPC-3 lysate were separated by SDS-PAGE on three replicate 12 percent polyacrylamide gels. Electrophoresis was carried out as described above. Proteins were transferred from the gel to PVDF membranes (Millipore, Billerica, Mass.) by electroblotting for 16 hours at 40 V. After transfer, the membranes were blocked with 5 percent skim milk powder in TBST for 2 hours. The membranes were probed with either AR40A746.2.3, IgG1 isotype control (clone 1B7.11) or anti-CD9 (clone MEM-61) diluted in 3 percent skim milk powder in TBST at a concentration of 5 micrograms/mL for 2 hours. After washing 3 times with TBST for 10 minutes each, the membranes were incubated with goat anti-mouse IgG (Fc) conjugated HRP for 1 hour. This incubation was followed by washing 3 times with TBST for 10 minutes each, followed by incubation with ECL solution for 5 minutes. The membranes were exposed to film, and the film developed. Results from the cross-immunoprecipitation Western blots are shown in FIG. 31. When AR40A746.2.3 was used as primary antibody on the Western blot (Panel A) it reacted strongly to its self-immunoprecipitate, as well as the anti-CD9 (clone MEM-61) immunoprecipitate and BxPC-3 lysate. There also appears to be a band at approximately 25 kDa in the IgG1 isotype control (clone 1B7.11) immunoprecipitate. However, this is most likely non-specific given that it is seen across all lanes, including the molecular weight standards. When anti-CD9 (clone MEM-61) was used as a primary antibody on the Western blot (Panel B), it reacted strongly with AR40A746.2.3, as well as detecting a band at approximately 25 kDa in its self-immunoprecipitate and in the BxPC-3 lysate. The Western blot probed with IgG1 isotype control (clone 1B7.11; panel C) had reactivity in higher molecular weight regions corresponding to sizes of contaminating antibody fragments in the immunoprecipitates, while there was no reactivity at the 25 kDa region in any sample. The results from the cross-immunoprecipitation Western blots demonstrate that AR40A746.2.3 immunoprecipitated protein is recognized by the anti-CD9 antibody (clone MEM-61), and that anti-CD9 (clone MEM-61) immunoprecipitate is recognized by AR40A746.2.3.

The mass spectroscopic identification combined with the confirmation using a known commercial antibody demonstrates that the antigen for AR40A746.2.3 is CD9.

### EXAMPLE 15

### Murine Sequence of AR40A746.2.3

### 1.0 Cloning Variable Region Genes into Sequencing Vectors

The genes encoding the variable regions of both heavy and light chains were separately cloned into the commercial sequencing vector pCR2.1 (Invitrogen, Burlington, Ontario).

1.1 Isolation of mRNA

Total ribonucleic acid (RNA) was isolated from a vial of frozen Master Cell Bank AR40A746.2.3 hybridoma cells using Absolutely RNA® Miniprep kit (Stratagene, La Jolla, Calif.). RNA was stored at −80° C. until required for further use.

### 1.2 RT-PCR and Amplification of Variable Region Genes

Separate reactions were carried out to amplify the light and heavy chain variable regions. Reverse transcriptase polymerase chain reaction (RT-PCR) synthesized complimentary deoxynucleic acid (cDNA) from the total RNA template, and then specifically amplified the targeted gene.

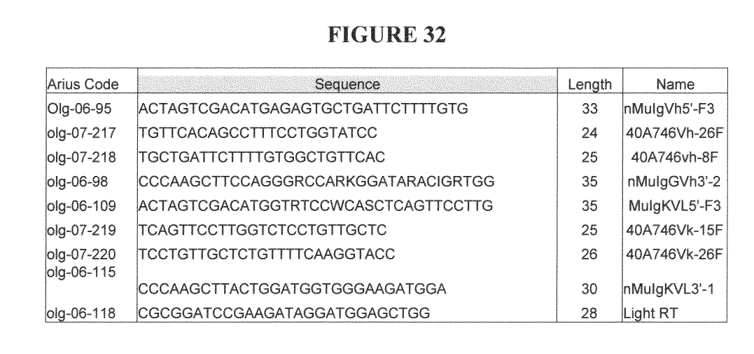

For both the light and heavy chains, one microgram of the total RNA was combined with 1 microliter of 10 millimolar deoxyribonucleotide triphosphates (dNTP), and 0.2 microliters of 10 micromolar primer. Light RT primer (Arius CODE:olg-06-118; FIG. 32) was used for the light chain reaction and nMuIgGVh3′-2 primer (Arius CODE:olg-06-98, FIG. 32) was used for the heavy chain reaction. The mixtures were incubated at 65° C. for 5 minutes, and then cooled on ice for one minute. First strand cDNA reactions were prepared using SuperScript III™ RT-PCR System (Invitrogen, Burlington, Ontario).

To amplify the variable region of the light chain or heavy chain, each PCR reaction contained 2 microliters of first strand cDNA prepared from the RT-PCR reaction, 5 microliters of 10× HI-FI PCR buffer (Invitrogen, Burlington, Ontario), 1.0 microliter of 25 micromolar dNTPs (Bio Basic Inc., Markham, Ontario), 1 microliter of 10 micromolar forward primer, 1 microliter of 10 micromolar reverse primer, 0.2 microliters of HI-FI Platinum Taq DNA Polymerase (Invitrogen, Burlington, Ontario) and 39.6 microliters of water.

For the light chain PCR, the reverse primer was either Light RT primer (Arius CODE:olg-06-118; FIG. 32) or nMulgKVL3′-1 (Arius CODE:olg-06-115; FIG. 32) and the forward primer was one of nMuIgKVL5′-F3 (Arius CODE:olg-06-109, FIG. 32), 40A746Vk-15F (Arius CODE:olg-06-219; FIG. 32) or 40A746Vk-26F (Arius CODE:olg-06-220; FIG. 32) primer.

To amplify the heavy chain variable region, the reverse primer was nMuIgGVh3′-2 primer (Arius CODE:olg-06-98, FIG. 32) and the forward primer was one of nMuIgVh5′-F3 (Arius CODE:olg-06-95, FIG. 32), 40A746Vh-26F (Arius CODE:olg-06-217; FIG. 32) or 40A746Vh-8F (Arius CODE:olg-06-218; FIG. 32) primer.

All PCR reactions were incubated in a thermocycler for 2 minutes at 95° C., followed by 30 cycles of 95° C. for 30 seconds, 55° C. for 2 minutes and 68° C. for 1 minute and a final incubation of 68° C. for 7 minutes. Reactions were stored at 4° C. until required. Ten microliters of each reaction was run on a 1.2 percent agarose gel and visualized with ethidium bromide under ultra-violet light.

The PCR products from the amplified light and heavy chain reactions were purified using QIAquick PCR Purification kit (QIAGEN, Mississauga, Ontario).

1.3 Cloning into Sequencing Vectors

Light and heavy chain purified PCR products were separately cloned into the pCR2.1 vector using the TOPO TA Cloning® Kit (Invitrogen, Burlington, Ontario). The reactions contained 4 microliters of purified PCR product. After ligation, 3 microliters were transformed into One Shot® MACH-1™-T1^{R} E. Coli (Invitrogen, Burlington, Ontario). Fifty microliters of the transformed cells were plated onto pre-warmed Lennox L broth (LB) agar (Sigma, Oakville, Ontario) plates containing 50 micrograms/mL ampicillin (Sigma, Oakville, Ontario) and 40 microliters of 40 mg/mL 5-bromo-4-chloro-3-indolyl-beta-D-galactopyranoside (X-gal, Calcdon Laboratories, Georgetown, Ontario) in N,N-dimethylformamide (Calcdon Laboratories, Georgetown, Ontario). The plates were inverted and incubated at 37° C. overnight.

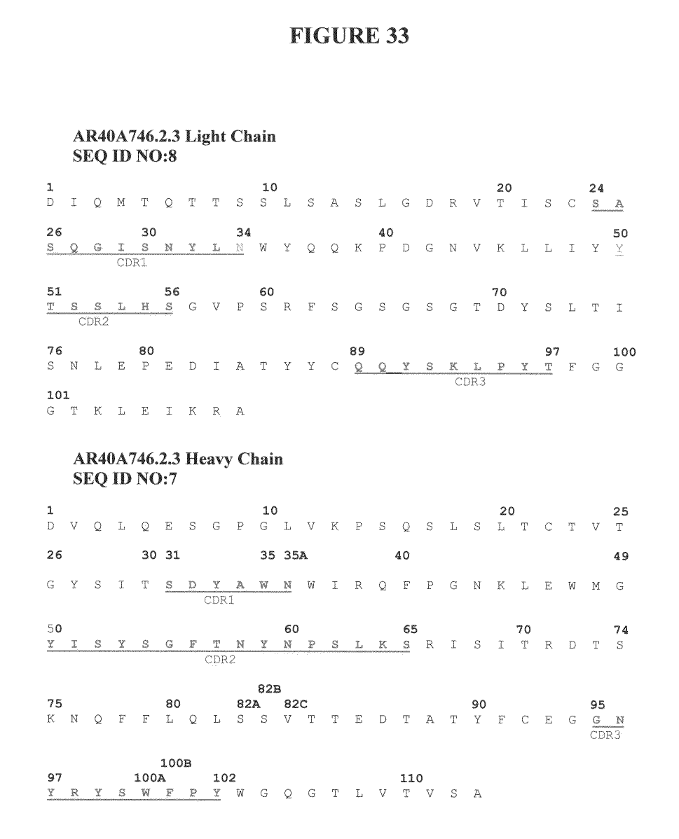

Four or more single white colonies with recombinant DNA from each transformed plate were used to inoculate cultures of 4 milliliters of LB broth containing 50 micrograms per milliliter of ampicillin overnight at 37° C. while shaking. The plasmids were isolated from these overnight cultures using QIAprep Spin Microprep kit (QIAGEN, Mississauga, Ontario). The plasmids with light chain (MBPP 953, 954, 956, 960, 961, 963, 965-973) or heavy chain (MBPP 991-1002) inserts were sequenced at Quintara (Berkeley, Calif., USA). The sequencing data was analyzed using Vector NTI software (Invitrogen, Burlington, Ontario) to obtain DNA and protein sequences. The light and heavy chain protein sequences are given as SEQ ID NO:8 and SEQ ID NO: 7 respectively (FIG. 33). The CDR regions and sequence numbering are given according to Kabat.

### EXAMPLE 16

### Isolation of Competitive Binders

Given an antibody, an individual ordinarily skilled in the art can generate a competitively inhibiting CDMAB, for example a competing antibody, which is one that recognizes the same epitope (Belanger L et al. Clinica Chimica Acta 48:15-18 (1973)). One method entails immunizing with an immunogen that expresses the antigen recognized by the antibody. The sample may include but is not limited to tissues, isolated protein(s) or cell line(s). Resulting hybridomas could be screened using a competition assay, which is one that identifies antibodies that inhibit the binding of the test antibody, such as ELISA, FACS or Western blotting. Another method could make use of phage display antibody libraries and panning for antibodies that recognize at least one epitope of said antigen (Rubinstein J L et al. Anal Biochem 314:294-300 (2003)). In either case, antibodies are selected based on their ability to displace the binding of the original labeled antibody to at least one epitope of its target antigen. Such antibodies would therefore possess the characteristic of recognizing at least one epitope of the antigen as the original antibody.

### EXAMPLE 17

### Cloning of the Variable Regions of the AR40A746.2.3 Monoclonal Antibody

The sequences of the variable regions from the heavy (V_{H}) and light (V_{L}) chains of monoclonal antibody produced by the AR40A746.2.3 hybridoma cell line were determined (as disclosed in Example 14). To generate chimeric and humanized IgG, the variable light and variable heavy domains can be subcloned into an appropriate vector for expression.

In another embodiment, AR40A746.2.3 or its de-immunized, chimeric or humanized version is produced by expressing a nucleic acid encoding the antibody in a transgenic animal, such that the antibody is expressed and can be recovered. For example, the antibody can be expressed in a tissue specific manner that facilitates recovery and purification. In one such embodiment, an antibody of the invention is expressed in the mammary gland for secretion during lactation. Transgenic animals include but are not limited to mice, goat and rabbit.

### (i) Monoclonal Antibody

DNA encoding the monoclonal antibody is readily isolated and sequenced using conventional procedures (e.g., by using oligonucleotide probes that are capable of binding specifically to genes encoding the heavy and light chains of the monoclonal antibodies). The hybridoma cell serves as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences. Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

### (ii) Humanized Antibody

A humanized antibody has one or more amino acid residues introduced into it from a non-human source. These non-human amino acid residues are often referred to as “import” residues, which are typically taken from an “import” variable domain. Humanization can be performed using the method of Winter and co-workers by substituting rodent CDRs or CDR sequences for the corresponding sequences of a human antibody (Jones et al., Nature 321:522-525 (1986); Riechmann et al., Nature 332:323-327 (1988); Verhoeyen et al., Science 239:1534-1536 (1988); reviewed in Clark, Immunol. Today 21:397-402 (2000)).

A humanized antibody can be prepared by a process of analysis of the parental sequences and various conceptual humanized products using three-dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

(iii) Antibody Fragments

Various techniques have been developed for the production of antibody fragments. These fragments can be produced by recombinant host cells (reviewed in Hudson, Curr. Opin. Immunol. 11:548-557 (1999); Little et al., Immunol. Today 21:364-370 (2000)). For example, Fab′-SH fragments can be directly recovered from E. coli and chemically coupled to form F(ab′)₂ fragments (Carter et al., Biotechnology 10:163-167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture.

### EXAMPLE 18

### Intracellular Kinase Proteome Profiler Blots

To identify intracellular signaling molecules affected by AR40A746.2.3 treatment, lysates from cells treated with AR40A746.2.3 were screened using a proteome profiler human phospho-MAPK antibody array (ARY002, R&D Systems Inc., Minneapolis, Minn.).

### Treatment and Preparation of Cells

Previous work demonstrated in vivo efficacy of AR40A746.2.3 in a pancreatic cancer xenograft model using BxPC-3 cells grown in severe combined immunodeficient (SCID) mice. Accordingly, screening for activation of intracellular signaling molecules was done using BxPC-3 cells lines. BxPC-3 cells were grown to near confluence, washed with phosphate buffered saline (PBS) and then starved in serum and supplement-deficient media for overnight at 37° C. After this, AR40A746.2.3 (20 micrograms/ml) or IB7.11 (IgG1) (20 micrograms/ml) was added to the cells and allowed to bind for 20 minutes at 4° C. Cells were then stimulated by adding fetal bovine serum (FBS), L-glutamine and sodium pyruvate to the cells to give a final concentration of 10 percent FBS, 1 percent L-glutamine, and 1 percent sodium pyruvate. The cells were placed in an incubator at 37° C. and the cell lysate was collected 1 hour after stimulation. Lysates were collected by washing the cells twice with PBS and harvesting in lysis buffer 6 (Part no. 895561: R&D Systems antibody array ARY002). The cells were resuspended by pipetting, transferred to a 1.5 ml microfuge tube and mixed by rotation at 4° C. for 30 minutes. Lysates were the centrifuged at 14000×g for five minutes and the supernatant was transferred to a clean tube. Protein concentration was determined by bicinchoninic acid (BCA) protein assay (Pierce, Rockford, Ill.).

### Human Phospho-MAPK Antibody Array

Human phospho-MAPK antibody array were screened with BxPC-3 cell lysates according to the protocol described by the manufacturer (Fourth Revision, May 2006, R&D Systems antibody array ARY002). Briefly, each human phospho-MAPK profiler membrane was prepared by incubating in 1.5 mls of array buffer 1 (Part no. 895477: R&D Systems antibody array ARY002) for 1 hour on a rocking platform shaker. For each treatment, 200 micrograms of total protein was diluted with lysis buffer 6 to give a final volume of 250 microliters and mixed with 1.25 mls of array buffer 1. This mixture was added to the prepared profiler membranes and incubated at 4° C. overnight on a rocking platform shaker. Each membrane was then washed 3 times in 1× wash buffer (diluted in purified distilled water from a 25× stock, (Part no. 895003: R&D Systems antibody array ARY002)) and incubated for 2 hours with 1.5 mls of anti-phospho-MAPK detection antibody cocktail (containing biotinylated phospho-specific antibodies) (Part no. 893051: R&D Systems antibody array ARY002) prepared in 1× array buffer 2/3 (5× array buffer 2, Part no. 895478: R&D Systems antibody array ARY002; array buffer 3, Part no. 895008: R&D Systems antibody array ARY002). The membranes were washed 3 times in 1× wash buffer and incubated for 30 minutes with 1.5 mls of Streptavidin-HRP (Part no. 890803: R&D Systems antibody array ARY002) diluted 1:2000 in 1× array buffer 2/3. The membranes were washed 3 times in 1× wash buffer and exposed to ECL plus Western detection reagents (GE Healthcare, Life Sciences, Piscataway, N.J.) for developing. Membranes were exposed to chemiluminescent film (Kodak, Cedex, France) and developed using an X-ray medical processor. Phospho-MAPK array data on developed X-ray films were quantitated by scanning the film on a transmission-mode scanner and analyzing the array image file using Image J analysis software (Image J1.37v, NIH). For each kinase, the average pixel density for corresponding duplicate spots was calculated and subtracted from background signal using the pixel density of a clear area on the membrane. The average normalized pixel density of AR40A746.2.3-treated samples was divided by the average normalized pixel density of 1B7.11 treated samples for each corresponding phospho-protein target to obtain a ratio of relative change. The percent reduction of phospho-protein signal was determined by subtracting the ratio of relative change from 1 and multiplying by 100.

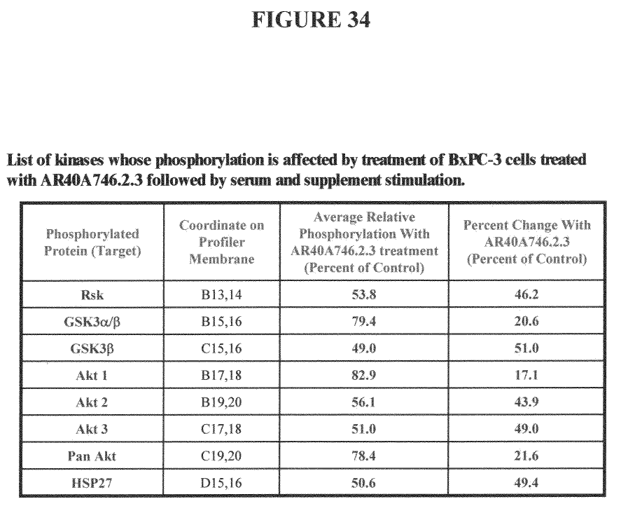

The results from phospho-MAPK array membranes showing changes in spot intensity as a percent reduction with AR40A746.2.3 are shown in FIG. 34. Compared with 1B7.11, AR40A746.2.3 suppressed the phosphorylation of 90 kDa ribosomal S6 kinase (Rsk) (46.2 percent), glycogen synthase kinase 3 alpha/beta (Gsk3α/β (20.6 percent); Gsk3β (51.0 percent)), Akt protein kinase B (PKB) (total Akt (pan Akt (21.6 percent), Akt1/PKBalpha (17.1 percent), Akt2/PKBbeta (43.9 percent) and Akt3/PKBgamma (49.0 percent)) and heat shock protein (HSP) 27 (49.4 percent) in BxPC-3 cells stimulated with serum and supplements. These kinases are involved in intracellular signaling pathways that can affect cell proliferation, growth and survival. That AR40A746.2.3 can reduce the phosphorylation of these kinases upon stimulation by serum and supplements suggest that AR40A746.2.3 may block cell growth and survival through these kinases and their related intracellular signaling pathways. Therefore, this data provides potential directions towards understanding mechanism of action for AR40A746.2.3 through intracellular signaling and identifying novel markers or indicators for measuring AR40A746.2.3 activity and for patient selection.

### EXAMPLE 19

### Receptor Tyrosine Kinase Proteome Profiler Blots

To identify intracellular signaling molecules affected by AR40A746.2.3 treatment, lysates from cells treated with AR40A746.2.3 were screened using a proteome profiler human phospho-RTK antibody array (ARY001, R&D Systems Inc., Minneapolis, Minn.).

### Treatment and Preparation of Cells

Previous work demonstrated in vivo efficacy of AR40A746.2.3 in a pancreatic cancer xenograft model using BxPC-3 cells grown in severe combined immunodeficient (SCID) mice. Accordingly, screening for activation of intracellular signaling molecules was done using BxPC-3 cells lines. BxPC-3 cells were grown to near confluence, washed with phosphate buffered saline (PBS) and then starved in serum and supplement-deficient media for overnight at 37° C. After this, AR40A746.2.3 (20 micrograms/mL) or 1B7.11 (IgG1) (20 micrograms/mL) was added to the cells and allowed to bind for 20 minutes at 4° C. Cells were then stimulated by adding fetal bovine serum (FBS), L-glutamine and sodium pyruvate to the cells to give a final concentration of 10 percent FBS, 1 percent L-glutamine, and 1 percent sodium pyruvate. The cells were placed in an incubator at 37° C. and the cell lysate was collected 15 minutes after stimulation. Lysates were collected by washing the cells twice with PBS and harvesting in NP-40 lysis buffer (1 percent NP-40, 20 mM Tris-HCl (pH 8.0), 137 mM NaCl, 10 percent glycerol, 2 mM EDTA, 1 mM sodium orthovanadate, 10 microgram/mL Aprotinin, 10 microgram/mL Leupeptin). The cells were resuspended by pipetting, transferred to a 1.5 mL microfuge tube and mixed by rotation at 4° C. for 30 minutes. Lysates were centrifuged at 14000×g for five minutes and the supernatant was transferred to a clean tube. Protein concentration was determined by bicinchoninic acid (BCA) protein assay (Pierce, Rockford, Ill.).

### Human Phospho-RTK Antibody Array

Human phospho-RTK antibody array were screened with BxPC-3 cell lysates according to the protocol described by the manufacturer (R&D Systems antibody array ARY001). Briefly, each human phospho-RTK profiler membrane was prepared by incubating in 1.5 mLs of array buffer 1 (Part no. 895477: R&D Systems antibody array ARY001) for 1 hour on a rocking platform shaker. For each treatment, a volume containing 200 micrograms of total protein was diluted to 1.5 mL with array buffer 1. This mixture was added to the prepared profiler membranes and incubated at 4° C. overnight on a rocking platform shaker. Each membrane was then washed 3 times in 1× wash buffer (diluted in purified distilled water from a 25× stock, (Part no. 895003: R&D Systems antibody array ARY001)) and incubated for 2 hours with 1.5 mLs of anti-phospho-tyrosine-HRP detection antibody (Part no. 841403: R&D Systems antibody array ARY001) prepared in 1× array buffer 2 (5× array buffer 2, Part no. 895478: R&D Systems antibody array ARY001). The membranes were washed 3 times in 1× wash buffer and exposed to ECL plus Western detection reagents (GE Healthcare, Life Sciences, Piscataway, N.J.) for developing. Membranes were exposed to chemiluminescent film (Kodak, Cedex, France) and developed using an X-ray medical processor. Phospho-RTK array data on developed X-ray films were quantitated by scanning the film on a transmission-mode scanner and analyzing the array image file using Image J analysis software (Image J1.37v, NIH). For each RTK, the average pixel density for corresponding duplicate spots was calculated and subtracted from background signal using the pixel density of a clear area on the membrane. The average normalized pixel density of AR40A746.2.3-treated samples was divided by the average normalized pixel density of 1B7.11 treated samples for each corresponding phospho-protein target to obtain a ratio of relative change. The percent reduction of phospho-protein signal was determined by subtracting the ratio of relative change from 1 and multiplying by 100.

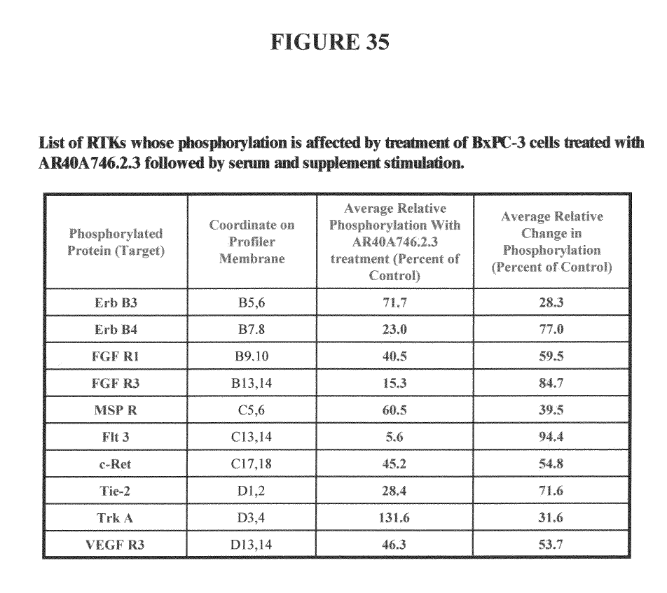

The results from phospho-RTK array membranes showing changes in spot intensity as a percent reduction with AR40A746.2.3 are shown in FIG. 35. Compared with 1B7.11, AR40A746.2.3 suppressed the phosphorylation of ErbB3 (HER3) (28.3 percent), ErbB4 (HER4) (77.0 percent), fibroblast growth factor (FGF) receptors 1 and 3 (FGF R1 (59.5 percent), FGF R3 (84.7 percent)), hepatocyte growth factor (HGF) receptor (MSP R) (39.5 percent), platelet derived growth factor (PDGF) receptor (Flt 3) (94.4 percent), c-RET (54.8 percent), Tie2/Tek (71.6 percent) and vascular endothelial growth factor (VEGF) receptor 3 (VEGF R3) (53.7 percent) in BxPC-3 cells stimulated with serum and supplements. Also, treatment with AR40A746.2.3 increased the phosphorylation of TrkA (31.6 percent) relative to treatment with isotype alone. These RTKs are involved in intracellular signaling pathways that can affect cell proliferation, growth and survival. That AR40A746.2.3 can affect the phosphorylation of these RTKs upon stimulation by serum and supplements suggest that AR40A746.2.3 may affect cell growth and survival through these RTKs and their related intracellular signaling pathways. Therefore, this data provides potential directions towards understanding mechanism of action for AR40A746.2.3 through intracellular signaling and identifying novel markers or indicators for measuring AR40A746.2.3 activity and for patient selection.

### EXAMPLE 20

### Annexin-V Staining of BxPC3 Cells that were Treated with mAR40A746.2.3

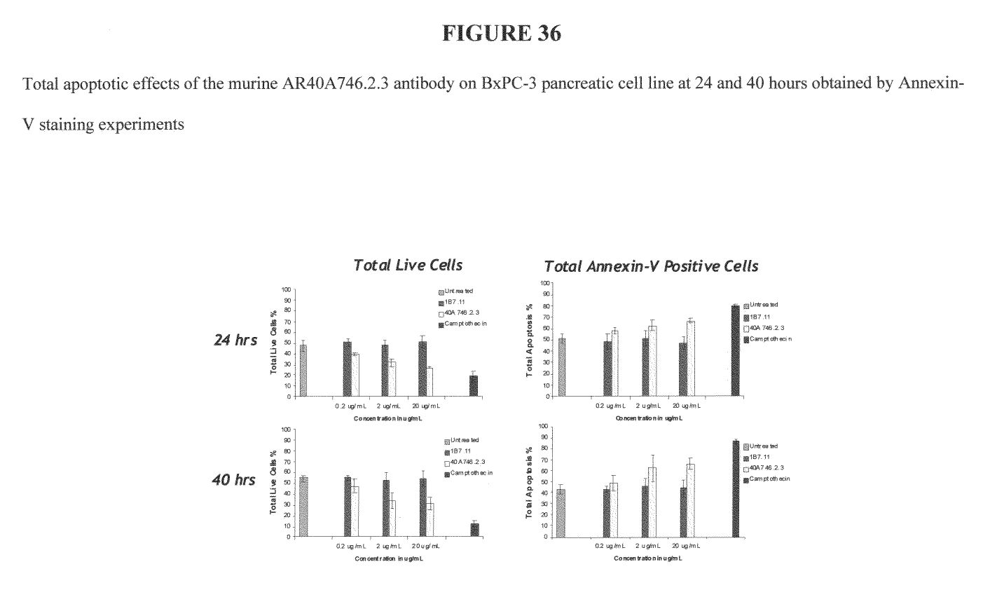

Annexin-V staining was performed to determine whether the murine antibody AR40A746.2.3 was able to induce apoptosis on the BxPC-3 human pancreatic cancer cell line. BxPC-3 cells were treated for 24 and 40 hours with AR40A746.2.3, at 0.2, 2 and 20 micrograms/mL. Each antibody concentration was tested in triplicate along with the appropriate isotype control (1B7.11, anti-TNP, murine IgG1, kappa, produced in-house) tested at the identical concentration. An untreated sample was included as the negative control and camptothecin (Biovision; Exton, Pa.) was included as the positive control. The FACS instrument was compensated for optical spillover of the fluorescent conjugates using fluorometric beads (BD Bioscience, Oakville, ON). The cells were then stained with Annexin-V and 7AAD and acquired on a FACS Array within 1 hour. Spontaneous apoptotic effects of cells treated with isotype control were found to be similar to cells treated with vehicle only. The murine AR40A746.2.3 antibody was found to induce apoptosis in the pancreatic cancer cell line in a dose dependent manner in each experiment, with greater apoptotic effect seen at a concentration of 20 μg/mL, were 61.3% of total apoptotic cells were obtained vs 36.1% obtained in cells treated with the isotype control (FIG. 36).

### EXAMPLE 21

### A Composition Comprising the Antibody of the Present Invention

The antibody of the present invention can be used as a composition for preventing/treating cancer. The composition for preventing/treating cancer, which comprises the antibody of the present invention, can be administered as they are in the form of liquid preparations, or as pharmaceutical compositions of suitable preparations to human or mammals (e.g., rats, rabbits, sheep, swine, bovine, feline, canine, simian, etc.) orally or parenterally (e.g., intravascularly, intraperitoneally, subcutaneously, etc.). The antibody of the present invention may be administered in itself, or may be administered as an appropriate composition. The composition used for the administration may contain a pharmacologically acceptable carrier with the antibody of the present invention or its salt, a diluent or excipient. Such a composition is provided in the form of pharmaceutical preparations suitable for oral or parenteral administration.

Examples of the composition for parenteral administration are injectable preparations, suppositories, etc. The injectable preparations may include dosage forms such as intravenous, subcutaneous, intracutaneous and intramuscular injections, drip infusions, intraarticular injections, etc. These injectable preparations may be prepared by methods publicly known. For example, the injectable preparations may be prepared by dissolving, suspending or emulsifying the antibody of the present invention or its salt in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mols) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is usually filled in an appropriate ampoule. The suppository used for rectal administration may be prepared by blending the antibody of the present invention or its salt with conventional bases for suppositories. The composition for oral administration includes solid or liquid preparations, specifically, tablets (including dragees and film-coated tablets), pills, granules, powdery preparations, capsules (including soft capsules), syrup, emulsions, suspensions, etc. Such a composition is manufactured by publicly known methods and may contain a vehicle, a diluent or excipient conventionally used in the field of pharmaceutical preparations. Examples of the vehicle or excipient for tablets are lactose, starch, sucrose, magnesium stearate, etc.

Advantageously, the compositions for oral or parenteral use described above are prepared into pharmaceutical preparations with a unit dose suited to fit a dose of the active ingredients. Such unit dose preparations include, for example, tablets, pills, capsules, injections (ampoules), suppositories, etc. The amount of the aforesaid compound contained is generally 5 to 500 mg per dosage unit form; it is preferred that the antibody described above is contained in about 5 to about 100 mg especially in the form of injection, and in 10 to 250 mg for the other forms.

The dose of the aforesaid prophylactic/therapeutic agent or regulator comprising the antibody of the present invention may vary depending upon subject to be administered, target disease, conditions, route of administration, etc. For example, when used for the purpose of treating/preventing, e.g., breast cancer in an adult, it is advantageous to administer the antibody of the present invention intravenously in a dose of about 0.01 to about 20 mg/kg body weight, preferably about 0.1 to about 10 mg/kg body weight and more preferably about 0.1 to about 5 mg/kg body weight, about 1 to 5 times/day, preferably about 1 to 3 times/day. In other parenteral and oral administration, the agent can be administered in a dose corresponding to the dose given above. When the condition is especially severe, the dose may be increased according to the condition.

The antibody of the present invention may be administered as it stands or in the form of an appropriate composition. The composition used for the administration may contain a pharmacologically acceptable carrier with the aforesaid antibody or its salts, a diluent or excipient. Such a composition is provided in the form of pharmaceutical preparations suitable for oral or parenteral administration (e.g., intravascular injection, subcutaneous injection, etc.). Each composition described above may further contain other active ingredients. Furthermore, the antibody of the present invention may be used in combination with other drugs, for example, alkylating agents (e.g., cyclophosphamide, ifosfamide, etc.), metabolic antagonists (e.g., methotrexate, 5-fluorouracil, etc.), anti-tumor antibiotics (e.g., mitomycin, adriamycin, etc.), plant-derived anti-tumor agents (e.g., vincristine, vindesine, Taxol, etc.), cisplatin, carboplatin, etoposide, irinotecan, etc. The antibody of the present invention and the drugs described above may be administered simultaneously or at staggered times to the patient.

The method of treatment described herein, particularly for cancers, may also be carried out with administration of other antibodies or chemotherapeutic agents. For example, an antibody against EGFR, such as ERBITUX® (cetuximab), may also be administered, particularly when treating colon cancer. ERBITUX® has also been shown to be effective for treatment of psoriasis. Other antibodies for combination use include Herceptin® (trastuzumab) particularly when treating breast cancer, AVASTIN® particularly when treating colon cancer and SGN-15 particularly when treating non-small cell lung cancer. The administration of the antibody of the present invention with other antibodies/chemotherapeutic agents may occur simultaneously, or separately, via the same or different route.

The chemotherapeutic agent/other antibody regimens utilized include any regimen believed to be optimally suitable for the treatment of the patient's condition. Different malignancies can require use of specific anti-tumor antibodies and specific chemotherapeutic agents, which will be determined on a patient to patient basis. In a preferred embodiment of the invention, chemotherapy is administered concurrently with or, more preferably, subsequent to antibody therapy. It should be emphasized, however, that the present invention is not limited to any particular method or route of administration.

The preponderance of evidence shows that AR40A746.2.3 mediates anti-cancer effects and prolongs survival through ligation of epitopes present on CD9. It has been shown (as disclosed in Example 13) that AR40A746.2.3 antibodies can be used to immunoprecipitate the cognate antigen from expressing cells such as BxPC-3 cells. Further it could be shown that AR40A746.2.3, chimeric AR40A746.2.3 or humanized variants can be used in the detection of cells and/or tissues which express a CD9 antigenic moiety which specifically binds thereto, utilizing techniques illustrated by, but not limited to FACS, cell ELISA or IHC.

As with the AR40A746.2.3 antibody, other anti-CD9 antibodies could be used to immunoprecipitate and isolate other forms of the CD9 antigen, and the antigen can also be used to inhibit the binding of those antibodies to the cells or tissues that express the antigen using the same types of assays.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objects and obtain the ends and advantages mentioned, as well as those inherent therein. Any oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. The isolated monoclonal antibody produced by the hybridoma deposited with the IDAC as accession number 141204-01.

2. A humanized antibody of the isolated monoclonal antibody produced by the hybridoma deposited with the IDAC as accession number 141204-01 or an antigen binding fragment produced from said humanized antibody.

3. A chimeric antibody of the isolated monoclonal antibody produced by the hybridoma deposited with the IDAC as accession number 141204-01 or an antigen binding fragment produced from said chimeric antibody.

4. The isolated hybridoma cell line deposited with the IDAC as accession number 141204-01.

5. A method for initiating antibody induced cytotoxicity of cancerous cells in a tissue sample selected from a human prostate, breast or pancreatic tumor comprising:
providing a tissue sample from said prostate, breast or pancreatic human tumor;
providing the isolated monoclonal antibody produced by the hybridoma deposited with the IDAC as accession number 141204-01, the humanized antibody of the isolated monoclonal antibody produced by the hybridoma deposited with the IDAC as accession number 141204-01, the chimeric antibody of the isolated monoclonal antibody produced by the hybridoma deposited with the IDAC as accession number 141204-01 or a CDMAB thereof, which CDMAB is characterized by an ability to competitively inhibit binding of said isolated monoclonal antibody to its target antigen; and
contacting said isolated monoclonal antibody, said humanized antibody, said chimeric antibody or CDMAB thereof with said tissue sample;
wherein binding of said isolated monoclonal antibody, said humanized antibody, said chimeric antibody or CDMAB thereof with said tissue sample induces cytotoxicity.

6. A CDMAB of the isolated monoclonal antibody of claim 1.

7. A CDMAB of the humanized antibody of claim 2.

8. A CDMAB of the chimeric antibody of claim 3.

9. The isolated antibody or CDMAB thereof, of any one of claims 1, 2, 3, 6, 7 or 8 conjugated with a member selected from the group consisting of cytotoxic moieties, enzymes, radioactive compounds, and hematogenous cells.

10. A method of reduction of a human prostate, breast or pancreatic tumor in a mammal, wherein said human prostate, breast or pancreatic tumor expresses at least one epitope of an antigen which specifically binds to the isolated monoclonal antibody encoded by a clone deposited with the IDAC as accession number 141204-01 or a CDMAB thereof, which CDMAB is characterized by an ability to competitively inhibit binding of said isolated monoclonal antibody to its target antigen, comprising administering to said mammal said monoclonal antibody or CDMAB thereof in an amount effective to result in a reduction of said mammal's prostate, breast or pancreatic tumor burden.

11. The method of claim 10 wherein said isolated monoclonal antibody is conjugated to a cytotoxic moiety.

12. The method of claim 11 wherein said cytotoxic moiety is a radioactive isotope.

13. The method of claim 10 wherein said isolated monoclonal antibody or CDMAB thereof activates complement.

14. The method of claim 10 wherein said isolated monoclonal antibody or CDMAB thereof mediates antibody dependent cellular cytotoxicity.

15. The method of claim 10 wherein said isolated monoclonal antibody is a humanized antibody of the isolated monoclonal antibody produced by the hybridoma deposited with the IDAC as accession number 141204-01 or an antigen binding fragment produced from said humanized antibody.

16. The method of claim 10 wherein said isolated monoclonal antibody is a chimeric antibody of the isolated monoclonal antibody produced by the hybridoma deposited with the IDAC as accession number 141204-01 or an antigen binding fragment produced from said chimeric antibody.

17. A method of reduction of a human prostate, breast or pancreatic tumor susceptible to antibody induced cellular cytotoxicity in a mammal, wherein said human prostate, breast or pancreatic tumor expresses at least one epitope of an antigen which specifically binds to the isolated monoclonal antibody encoded by a clone deposited with the IDAC as accession number 141204-01 or a CDMAB thereof, which CDMAB is characterized by an ability to competitively inhibit binding of said isolated monoclonal antibody to its target antigen, comprising administering to said mammal said monoclonal antibody or said CDMAB thereof in an amount effective to result in a reduction of said mammal's prostate, breast or pancreatic tumor burden.

18. The method of claim 17 wherein said isolated monoclonal antibody is conjugated to a cytotoxic moiety.

19. The method of claim 18 wherein said cytotoxic moiety is a radioactive isotope.

20. The method of claim 17 wherein said isolated monoclonal antibody or CDMAB thereof activates complement.

21. The method of claim 17 wherein said isolated monoclonal antibody or CDMAB thereof mediates antibody dependent cellular cytotoxicity.

22. The method of claim 17 wherein said isolated monoclonal antibody is a humanized antibody of the isolated monoclonal antibody produced by the hybridoma deposited with the IDAC as accession number 141204-01 or an antigen binding fragment produced from said humanized antibody.

23. The method of claim 17 wherein said isolated monoclonal antibody is a chimeric antibody of the isolated monoclonal antibody produced by the hybridoma deposited with the IDAC as accession number 141204-01 or an antigen binding fragment produced from said chimeric antibody.

24. A method of reduction of a human prostate, breast or pancreatic tumor in a mammal, wherein said human prostate, breast or pancreatic tumor expresses at least one epitope of an antigen which specifically binds to the isolated monoclonal antibody produced by the hybridoma deposited with the IDAC as accession number 141204-01 or a CDMAB thereof, which CDMAB is characterized by an ability to competitively inhibit binding of said isolated monoclonal antibody to its target antigen, comprising administering to said mammal said monoclonal antibody or CDMAB thereof in conjunction with at least one chemotherapeutic agent in an amount effective to result in a reduction of said mammal's prostate, breast or pancreatic tumor burden.

25. The method of claim 24 wherein said isolated monoclonal antibody is conjugated to a cytotoxic moiety.

26. The method of claim 25 wherein said cytotoxic moiety is a radioactive isotope.

27. The method of claim 24 wherein said isolated monoclonal antibody or CDMAB thereof activates complement.

28. The method of claim 24 wherein said isolated monoclonal antibody or CDMAB thereof mediates antibody dependent cellular cytotoxicity.

29. The method of claim 24 wherein said isolated monoclonal antibody is a humanized antibody of the isolated monoclonal antibody produced by the hybridoma deposited with the IDAC as accession number 141204-01 or an antigen binding fragment produced from said humanized antibody.

30. The method of claim 24 wherein said isolated monoclonal antibody is a chimeric antibody of the isolated monoclonal antibody produced by the hybridoma deposited with the IDAC as accession number 141204-01 or an antigen binding fragment produced from said chimeric antibody.

31. Use of monoclonal antibodies for reduction of human prostate, breast or pancreatic tumor burden, wherein said human prostate, breast or pancreatic tumor expresses at least one epitope of an antigen which specifically binds to the isolated monoclonal antibody produced by the hybridoma deposited with the IDAC as accession number 141204-01 or a CDMAB thereof, which CDMAB is characterized by an ability to competitively inhibit binding of said isolated monoclonal antibody to its target antigen, comprising administering to said mammal said monoclonal antibody or CDMAB thereof in an amount effective to result in a reduction of said mammal's human prostate, breast or pancreatic tumor burden.

32. The method of claim 31 wherein said isolated monoclonal antibody is conjugated to a cytotoxic moiety.

33. The method of claim 32 wherein said cytotoxic moiety is a radioactive isotope.

34. The method of claim 31 wherein said isolated monoclonal antibody or CDMAB thereof activates complement.

35. The method of claim 31 wherein said isolated monoclonal antibody or CDMAB thereof mediates antibody dependent cellular cytotoxicity.

36. The method of claim 31 wherein said isolated monoclonal antibody is a humanized antibody of the isolated monoclonal antibody produced by the hybridoma deposited with the IDAC as accession number 141204-01.

37. The method of claim 31 wherein said isolated monoclonal antibody is a chimeric antibody of the isolated monoclonal antibody produced by the hybridoma deposited with the IDAC as accession number 141204-01.

38. Use of monoclonal antibodies for reduction of human prostate, breast or pancreatic tumor burden, wherein said human prostate, breast or pancreatic tumor expresses at least one epitope of an antigen which specifically binds to the isolated monoclonal antibody produced by the hybridoma deposited with the IDAC as accession number 141204-01 or a CDMAB thereof, which CDMAB is characterized by an ability to competitively inhibit binding of said isolated monoclonal antibody to its target antigen, comprising administering to said mammal said monoclonal antibody or CDMAB thereof; in conjunction with at least one chemotherapeutic agent in an amount effective to result in a reduction of said mammal's human prostate, breast or pancreatic tumor burden.

39. The method of claim 38 wherein said isolated monoclonal antibody is conjugated to a cytotoxic moiety.

40. The method of claim 39 wherein said cytotoxic moiety is a radioactive isotope.

41. The method of claim 38 wherein said isolated monoclonal antibody or CDMAB thereof activates complement.

42. The method of claim 38 wherein said isolated monoclonal antibody or CDMAB thereof mediates antibody dependent cellular cytotoxicity.

43. The method of claim 38 wherein said isolated monoclonal antibody is a humanized antibody of the isolated monoclonal antibody produced by the hybridoma deposited with the IDAC as accession number 141204-01.

44. The method of claim 38 wherein said isolated monoclonal antibody is a chimeric antibody of the isolated monoclonal antibody produced by the hybridoma deposited with the IDAC as accession number 141204-01.

45. A process for reduction of a human prostate, breast or pancreatic tumor which expresses at least one epitope of human CD9 antigen which is specifically bound by the isolated monoclonal antibody produced by hybridoma cell line AR40A746.2.3 having IDAC Accession No. 141204-01, comprising:
administering to an individual suffering from said human tumor, at least one isolated monoclonal antibody or CDMAB thereof that binds the same epitope or epitopes as those bound by the isolated monoclonal antibody produced by the hybridoma cell line AR40A746.2.3 having IDAC Accession No. 141204-01;
wherein binding of said epitope or epitopes results in a reduction of prostate, breast or pancreatic tumor burden.

46. A process for reduction of a human prostate, breast or pancreatic tumor which expresses at least one epitope of human CD9 antigen which is specifically bound by the isolated monoclonal antibody produced by hybridoma cell line AR40A746.2.3 having IDAC Accession No. 141204-01, comprising:
administering to an individual suffering from said human tumor, at least one isolated monoclonal antibody or CDMAB thereof, that binds the same epitope or epitopes as those bound by the isolated monoclonal antibody produced by the hybridoma cell line AR40A746.2.3 having IDAC Accession No. 141204-01; in conjunction with at least one chemotherapeutic agent;
wherein said administration results in a reduction of prostate, breast or pancreatic tumor burden.

47. A binding assay to determine a presence of cancerous cells in a tissue sample selected from a human tumor, which is specifically bound by the isolated monoclonal antibody produced by hybridoma cell line AR40A746.2.3 having IDAC Accession No. 141204-01, the humanized antibody of the isolated monoclonal antibody produced by the hybridoma deposited with the IDAC as accession number 141204-01 or the chimeric antibody of the isolated monoclonal antibody produced by the hybridoma deposited with the IDAC as accession number 141204-01, comprising:
providing a tissue sample from said human tumor;
providing at least one of said isolated monoclonal antibody, said humanized antibody, said chimeric antibody or CDMAB thereof that recognizes the same epitope or epitopes as those recognized by the isolated monoclonal antibody produced by a hybridoma cell line AR40A746.2.3 having IDAC Accession No. 141204-01;
contacting at least one said provided antibodies or CDMAB thereof with said tissue sample; and determining binding of said at least one provided antibody or CDMAB thereof with said tissue sample;
whereby the presence of said cancerous cells in said tissue sample is indicated.

48. A binding assay to determine the presence of cells which express CD9 which is specifically recognized by the isolated monoclonal antibody produced by the hybridoma cell line AR40A746.2.3 having IDAC Accession No. 141204-01, the humanized antibody of the isolated monoclonal antibody produced by the hybridoma deposited with the IDAC as accession number 141204-01 or the chimeric antibody of the isolated monoclonal antibody produced by the hybridoma deposited with the IDAC as accession number 141204-01, comprising:
providing a cell sample;
providing the isolated monoclonal antibody produced by the hybridoma cell line AR40A746.2.3 having IDAC Accession No. 141204-01, said humanized antibody, said chimeric antibody or CDMAB thereof;
contacting said isolated monoclonal antibody or said antigen binding fragment with said cell sample; and
determining binding of said isolated monoclonal antibody or CDMAB thereof with said cell sample;
whereby the presence of cells which express an antigen of CD9 which is specifically bound by said isolated monoclonal antibody or said CDMBA thereof is determined.

49. A binding assay to determine the presence of primate cells which express CD9 which is specifically recognized by the isolated monoclonal antibody produced by the hybridoma cell line AR40A746.2.3 having IDAC Accession No. 141204-01, the humanized antibody of the isolated monoclonal antibody produced by the hybridoma deposited with the IDAC as accession number 141204-01 or the chimeric antibody of the isolated monoclonal antibody produced by the hybridoma deposited with the IDAC as accession number 141204-01, comprising:
providing a primate cell sample;
providing the isolated monoclonal antibody produced by the hybridoma cell line AR40A746.2.3 having IDAC Accession No. 141204-01, said humanized antibody, said chimeric antibody or CDMAB thereof;
contacting said isolated monoclonal antibody or said antigen binding fragment with said primate cell sample; and
determining binding of said isolated monoclonal antibody or CDMAB thereof with said primate cell sample;
whereby the presence of primate cells which express an antigen of CD9 which is specifically bound by said isolated monoclonal antibody or said CDMBA thereof is determined.

50. A binding assay to determine the presence of rabbit cells which express CD9 which is specifically recognized by the isolated monoclonal antibody produced by the hybridoma cell line AR40A746.2.3 having IDAC Accession No. 141204-01, the humanized antibody of the isolated monoclonal antibody produced by the hybridoma deposited with the IDAC as accession number 141204-01 or the chimeric antibody of the isolated monoclonal antibody produced by the hybridoma deposited with the IDAC as accession number 141204-01, comprising:
providing a rabbit cell sample;
providing the isolated monoclonal antibody produced by the hybridoma cell line AR40A746.2.3 having IDAC Accession No. 141204-01, said humanized antibody, said chimeric antibody or CDMAB thereof;
contacting said isolated monoclonal antibody or said antigen binding fragment with said rabbit cell sample; and
determining binding of said isolated monoclonal antibody or CDMAB thereof with said rabbit cell sample;
whereby the presence of rabbit cells which express an antigen of CD9 which is specifically bound by said isolated monoclonal antibody or said CDMBA thereof is determined.

51. A monoclonal antibody which specifically binds to the same epitope or epitopes as the isolated monoclonal antibody produced by the hybridoma deposited with the IDAC as accession number 141204-01.

52. An isolated monoclonal antibody or CDMAB thereof, which specifically binds to human CD9, in which the isolated monoclonal antibody or CDMAB thereof reacts with the same epitope or epitopes of human CD9 as the isolated monoclonal antibody produced by a hybridoma cell line AR40A746.2.3 having IDAC Accession No. 141204-01; said isolated monoclonal antibody or CDMAB thereof being characterized by an ability to competitively inhibit binding of said isolated monoclonal antibody to its target human CD9 antigen.

53. An isolated monoclonal antibody or CDMAB thereof that recognizes the same epitope or epitopes as those recognized by the isolated monoclonal antibody produced by the hybridoma cell line AR40A746.2.3 having IDAC Accession No. 141204-01; said monoclonal antibody or CDMAB thereof being characterized by an ability to competitively inhibit binding of said isolated monoclonal antibody to its target epitope or epitopes.

54. A monoclonal antibody that specifically binds the same epitope or epitopes of human CD9 as the isolated monoclonal antibody produced by the hybridoma cell line AR40A746.2.3 having IDAC Accession No. 141204-01, comprising:
a heavy chain variable region comprising the complementarity determining region amino acid sequences of SEQ ID NO:1, SEQ ID NO:2, and SEQ ID NO:3; and a light chain variable region comprising the complementarity determining region amino acid sequences of SEQ ID NO:4, SEQ ID NO:5, or SEQ ID NO:6;
or a human CD9 binding fragment thereof.

55. A monoclonal antibody that specifically binds the same epitope or epitopes of human CD9 as the isolated monoclonal antibody produced by the hybridoma cell line AR40A746.2.3 having IDAC Accession No. 141204-01, comprising:
a heavy chain variable region comprising the complementarity determining region amino acid sequences of SEQ ID NO:1, SEQ ID NO:2, and SEQ ID NO:3; and a light chain variable region comprising the complementarity determining region amino acid sequences of SEQ ID NO:4, SEQ ID NO:5, or SEQ ID NO:6; and variable domain framework regions from the heavy and light chains of a human antibody or human antibody consensus framework;
or a human CD9 binding fragment thereof.

56. A monoclonal antibody that specifically binds human CD9, wherein said monoclonal antibody comprises a heavy chain variable region amino acid sequence of SEQ ID NO:7; and a light chain variable region amino acid sequence selected of SEQ ID NO:8;
or a human CD9 binding fragment thereof.

57. A humanized antibody that specifically binds the same epitope or epitopes of human CD9 as the isolated monoclonal antibody produced by the hybridoma cell line AR40A746.2.3 having IDAC Accession No. 141204-01, comprising:
a heavy chain variable region comprising the complementarity determining region amino acid sequences of SEQ ID NO:1, SEQ ID NO:2, and SEQ ID NO:3; and a light chain variable region comprising the complementarity determining region amino acid sequences of SEQ ID NO:4, SEQ ID NO:5, or SEQ ID NO:6;
or a human CD9 binding fragment thereof.

58. A humanized antibody that specifically binds the same epitope or epitopes of human CD9 as the isolated monoclonal antibody produced by the hybridoma cell line AR40A746.2.3 having IDAC Accession No. 141204-01, comprising:
a heavy chain variable region comprising the complementarity determining region amino acid sequences of SEQ ID NO:1, SEQ ID NO:2, and SEQ ID NO:3; and a light chain variable region comprising the complementarity determining region amino acid sequences of SEQ ID NO:4, SEQ ID NO:5, or SEQ ID NO:6; and variable domain framework regions from the heavy and light chains of a human antibody or human antibody consensus framework;
or a human CD9 binding fragment thereof.

59. A humanized antibody that specifically binds human CD9, wherein said monoclonal antibody comprises a heavy chain variable region amino acid sequence of SEQ ID NO:7; and a light chain variable region amino acid sequence selected of SEQ ID NO:8;
or a human CD9 binding fragment thereof.

60. A composition effective for treating a human pancreatic, prostate, ovarian, breast or colon tumor comprising in combination:
an antibody or CDMAB of any one of claims 1, 2, 3, 6, 7, 8, 17, 49, 50, 54, 55, or 56;
a conjugate of said antibody or an antigen binding fragment thereof with a member selected from the group consisting of cytotoxic moieties, enzymes, radioactive compounds, cytokines, interferons, target or reporter moieties and hematogenous cells; and
a requisite amount of a pharmacologically acceptable carrier;
wherein said composition is effective for treating said human prostate, breast or pancreatic tumor.

61. A composition effective for treating a human prostate, breast or pancreatic tumor comprising in combination:
an antibody or CDMAB of any one of claims 1, 2, 3, 6, 7, 8, 17, 49, 50, 54, 55, or 56; and a requisite amount of a pharmacologically acceptable carrier;
wherein said composition is effective for treating said human prostate, breast or pancreatic tumor.

62. A composition effective for treating a human prostate, breast or pancreatic tumor comprising in combination:
a conjugate of an antibody, antigen binding fragment, or CDMAB of any one of claims 1, 2, 3, 6, 7, 8, 17, 49, 50, 54, 55, or 56; with a member selected from the group consisting of cytotoxic moieties, enzymes, radioactive compounds, cytokines, interferons, target or reporter moieties and hematogenous cells; and
a requisite amount of a pharmacologically acceptable carrier;
wherein said composition is effective for treating said human prostate, breast or pancreatic tumor.

63. An assay kit for detecting the presence of a human cancerous tumor, wherein said human cancerous tumor expresses at least one epitope of an antigen which specifically binds to the isolated monoclonal antibody produced by the hybridoma deposited with the IDAC as accession number 141204-01 or a CDMAB thereof, which CDMAB is characterized by an ability to competitively inhibit binding of said isolated monoclonal antibody to its target antigen, the kit comprising the isolated monoclonal antibody produced by the hybridoma deposited with the IDAC as accession number 141204-01 or a CDMAB thereof, and means for detecting whether the monoclonal antibody, or a CDMAB thereof, is bound to a polypeptide whose presence, at a particular cut-off level, is diagnostic of said presence of said human cancerous tumor.


SEQUENCE LISTING (17 sequences)

SEQ ID NO: 1 - protein, 6 aa, Artificial Sequence
  Description of Artificial Sequence Synthetic peptide
SDYAWN

SEQ ID NO: 2 - protein, 16 aa, Artificial Sequence
  Description of Artificial Sequence Synthetic peptide
YISYSGFTNYNPSLKS

SEQ ID NO: 3 - protein, 10 aa, Artificial Sequence
  Description of Artificial Sequence Synthetic peptide
GNYRYSWFPY

SEQ ID NO: 4 - protein, 11 aa, Artificial Sequence
  Description of Artificial Sequence Synthetic peptide
SASQGISNYLN

SEQ ID NO: 5 - protein, 7 aa, Artificial Sequence
  Description of Artificial Sequence Synthetic peptide
YTSSLHS

SEQ ID NO: 6 - protein, 9 aa, Artificial Sequence
  Description of Artificial Sequence Synthetic peptide
QQYSKLPYT

SEQ ID NO: 7 - protein, 119 aa, Artificial Sequence
  Description of Artificial Sequence Synthetic polypeptide
DVQLQESGPGLVKPSQSLSLTCTVTGYSITSDYAWNWIRQFPGNKLEWMGYISYSGFTNYNPSLKSRISITRDTSKNQFFLQLSSVTTEDTATYFCEGGNYRYSWFPYWGQGTLVTVSA

SEQ ID NO: 8 - protein, 109 aa, Artificial Sequence
  Description of Artificial Sequence Synthetic polypeptide
DIQMTQTTSSLSASLGDRVTISCSASQGISNYLNWYQQKPDGNVKLLIYYTSSLHSGVPSRFSGSGSGTDYSLTISNLEPEDIATYYCQQYSKLPYTFGGGTKLEIKRA

SEQ ID NO: 9 - DNA, 33 bp, Artificial Sequence
  Description of Artificial Sequence Synthetic primer
actagtcgacatgagagtgctgattcttttgtg

SEQ ID NO: 10 - DNA, 24 bp, Artificial Sequence
  Description of Artificial Sequence Synthetic primer
tgttcacagcctttcctggtatcc

SEQ ID NO: 11 - DNA, 25 bp, Artificial Sequence
  Description of Artificial Sequence Synthetic primer
tgctgattcttttgtggctgttcac

SEQ ID NO: 12 - DNA, 35 bp, Artificial Sequence
  Description of Artificial Sequence Synthetic primer
cccaagcttccagggrccarkggataracngrtgg

SEQ ID NO: 13 - DNA, 35 bp, Artificial Sequence
  Description of Artificial Sequence Synthetic primer
actagtcgacatggtrtccwcasctcagttccttg

SEQ ID NO: 14 - DNA, 25 bp, Artificial Sequence
  Description of Artificial Sequence Synthetic primer
tcagttccttggtctcctgttgctc

SEQ ID NO: 15 - DNA, 26 bp, Artificial Sequence
  Description of Artificial Sequence Synthetic primer
tcctgttgctctgttttcaaggtacc

SEQ ID NO: 16 - DNA, 30 bp, Artificial Sequence
  Description of Artificial Sequence Synthetic primer
cccaagcttactggatggtgggaagatgga

SEQ ID NO: 17 - DNA, 28 bp, Artificial Sequence
  Description of Artificial Sequence Synthetic primer
cgcggatccgaagataggatggagctgg